SEQUENCE LISTING (1 sequence)

SEQ ID NO: 1 - protein, 585 aa, Homo sapiens [sequence omitted]

(19) United States
(12) Patent Application Publication
(10) Pub. No.: US 2023/0000774 A1
(43) Pub. Date: Jan. 5, 2023

(54) METHODS AND COMPOSITIONS PRODUCED THEREBY

(71) Applicant: Albumedix Limited, Nottingham (GB)

(72) Inventors: Richard Alan Johnson, Nottingham (GB); Andrew Naylor, Nottingham (GB); Nicholas Jon Arrowsmith, Nottingham (GB); Iona Mary Munro, Nottingham (GB)

(21) Appl. No.: 17/780,569

(22) PCT Filed: Dec. 4, 2020
(86) PCT No.: PCT/GB2020/053108

§ 371 Date: May 27, 2022

(30) Foreign Application Priority Data
Dec. 4, 2019 (DK) PA 2019 70747

(51) Int. Cl. A61K 9/16; A61K 31/522; A61K 31/496; A61K 31/513; A61K 31/427; A61K 31/4174; A61K 31/505; A61K 31/397; A61K 31/64; A61K 31/4166

(52) U.S. Cl. CPC A61K 9/1658; A61K 9/1694; A61K 9/1623; A61K 31/522; A61K 31/496; A61K 31/513; A61K 31/427; A61K 31/4174; A61K 31/505; A61K 31/397; A61K 31/64; A61K 31/4166

(57) ABSTRACT

The invention relates to a method of enhancing the solubility and/or the rate of dissolution of a Class II or Class IV low solubility molecule, a method of producing a spray-dried composition, and a spray-dried composition comprising a Class II or Class IV low solubility molecule, albumin and an agent that prevents self-aggregation of albumin.

The present invention relates generally to methods for enhancing the solubility and/or the rate of dissolution of low solubility molecules, and in particular to enhancing the solubility and/or the rate of dissolution of Class II or Class IV molecules. The invention also relates to compositions comprising low solubility molecules, the solubility and/or the rate of dissolution of which has been enhanced.

### BACKGROUND OF THE INVENTION

Many new molecules emerging from pharmaceutical research laboratories suffer from low solubility (Class II and Class IV compounds), for example in aqueous solvents. These include both small molecules and larger peptides. This is a growing trend that is likely to continue in coming years, and it is estimated that over 90% of new chemical entities (NCEs) can be classified as Class II or Class IV compounds (Patel et al 2011, Int J Pharmaceut Biolog Arch 2:621-629).

Due to their low solubility in water these drugs are extremely hard to formulate in ways that are suitable for human administration.

The pharmaceutical industry has utilised a wide range of technologies to attempt to enhance solubility and/or the rate of dissolution of active pharmaceutical ingredients (APIs). Examples of enhancement technologies include micronisation, nanomilling, use of oils and surfactants, amorphous dispersions (e.g. spray drying and hot melt extrusion) and co-crystals. However, most of this work has been focused on oral formulation and delivery and would not be suitable for parenteral administration, e.g. intravenous administration.

Existing technologies for enhancing solubility and/or the rate of dissolution include the use of cyclodextrins and/or surfactants. However, these have the potential for side effects and so are not desirable for use in some parenteral routes of administration, particularly intravenous administration. For example, the poorly-soluble cancer drug Paclitaxel has been formulated in Cremophor EL® (a polyethoxylated castor oil, also known as Kolliphor® EL) for intravenous administration but a well-known side effect of this is anaphylaxis (Gelderblom et at 2001, Eur J Cancer 37(13):1590-1598). Other poorly-soluble drugs have been formulated with Solutol (polyethylene glycol (15)-hydroxystearate; also known as Kolliphor® HS 15) but these have been shown to cause side effects in dogs and humans (Stokes et al 2013, Int J Toxicol 32(3):189-197).

The problem of formulating low solubility drugs for parenteral administration, e.g. intravenous administration, is more difficult than that of formulating low solubility drugs for enteral administration, e.g. oral administration, because the pharmaceutical standards required for oral compositions are lower than those for intravenous compositions. Reasons for this include: (1) oral compositions require absorption of their components in order for them to enter the blood stream unlike the direct delivery of the entirety of intravenous compositions in their intact state; (2) oral compositions usually transit through the stomach etc. and are therefore exposed to low pH and digestive enzymes before most absorption takes place; and (3) the blood stream has a higher concentration of antibodies and immune system components than the gastrointestinal tract and therefore is more vulnerable to the introduction of immunogenic components of formulations. Therefore, subjects can usually tolerate a lower quality of composition via the enteral route than via the parenteral route.

The methods of the present invention, the spray-dried compositions produced by the methods of the present invention, and the related spray-dried compositions of the present invention, utilise albumin, such as recombinant human albumin, to make a spray-dried composition containing a low solubility molecule, e.g. an API.

Surprisingly, the spray-dried compositions produced by the methods of the present invention, and the related spray-dried compositions of the present invention, may by virtue of the albumin component be highly soluble in one or more aqueous solvents e.g. water, and have an increased rate of dissolution in one or more aqueous solvents e.g. water, without requiring additional solubility enhancing agents such as surfactants. Therefore, among other advantages, the present invention solves the problem of improving the solubility of insoluble or low solubility molecules, such as APIs, in water and other aqueous solvents.

Further, the spray-dried compositions described herein may be suitable for parenteral administration, such as intravenous administration. The albumin present in the spray-dried compositions described herein (including those produced by the methods described herein) may be substantially free (or within tolerance limits) of aggregation, denaturation and/or cross-linking, and thereby avoid the problems of immunogenicity. The spray-dried compositions described herein (including those produced by the methods described herein) may also be of low toxicity. Together, these characteristics can make the spray-dried compositions described herein suitable for parenteral administration, such as intravenous administration. It is expected that the delivery and/or bioavailability (for any non-intravenous administration) of the low solubility molecule will also be enhanced.

Spray drying also has several differences/advantages over freeze drying, including:
(i) the ability with spray drying to go from liquid to dry powder in a single, quick step, which is important for creating a uniform dry powder, whereas in freeze drying a frozen solution is formed, which can lead to partitioning;
(ii) the ability with spray drying to work with non-aqueous solvents, which is either much more difficult or impossible with freeze drying, depending on the solvent;
(iii) spray drying has no freezing step, and therefore the method avoids ice formation which can otherwise damage some proteins;
(iv) spray drying is much cheaper than freeze drying (cost advantage); and
(v) spray drying can produce large quantities of powder in a time scale of minutes rather than a time scale of days for freeze drying (scale advantage).

The present approach, as illustrated in the examples, is advantageous in utilising the following properties of albumin, spray drying and an agent that prevents self-aggregation of albumin:
the ability for albumin to be dissolved in mixtures of water and a water-miscible solvent such as ethanol to create a single-phase solution of a low solubility molecule, albumin and an agent that prevents self-aggregation of albumin such as trehalose for spray drying;
the rapid evaporative properties of spray drying may create a stable, molecular dispersion consisting of an amorphous mix of a low solubility molecule, albumin and an agent that prevents self-aggregation of albumin such as trehalose;
the ability of albumin to bind or hydrogen bond with low solubility molecules such as poorly soluble drugs to create stable nanocomplexes when the spray-dried powder is dissolved in water or other aqueous solvents, which nanocomplexes may enhance the solubility of the low solubility molecule, e.g. in aqueous solvents, and may also enhance the bioavailability of the drug when administered parenterally;
the ability of albumin to survive spray drying intact, by using a stable formulation and suitable spray drying conditions;
the suitability of albumin for parenteral delivery due to its biocompatibility, having no side effects compared to cyclodextrins or surfactants;
an agent that prevents self-aggregation of albumin such as trehalose may stabilise albumin in the dry state thus preventing polymerisation, and may also aid in increasing the solubility of the low solubility molecule when the spray-dried powder is dissolved in aqueous solvents e.g. water.

### DETAILED DESCRIPTION OF THE INVENTION

A first aspect of the invention provides a method of enhancing the solubility and/or the rate of dissolution of a Class II or Class IV low solubility molecule, the method comprising spray-drying a mixture comprising the Class II or Class IV low solubility molecule, a water-miscible solvent, albumin and an agent that prevents self-aggregation of albumin.

It will be appreciated that the order for combining the components of the mixture prior to spray-drying may be varied. Without limiting to particular embodiments, some example embodiments for different orders of combining the components are provided immediately below.

In one embodiment, the method comprises (a) dissolving the Class II or Class IV low solubility molecule in a water-miscible solvent to form a solution, (b) mixing the solution of the Class II or Class IV low solubility molecule and water-miscible solvent with albumin and an agent that prevents self-aggregation of albumin, and (c) spray-drying the mixture. It will be understood that step (b) may comprise sub-steps wherein either the albumin or the agent that prevents self-aggregation of albumin is added first, with optional mixing, followed by the addition of the other one of the albumin or the agent that prevents self-aggregation of albumin, followed by mixing.

In another embodiment, the method comprises (a) mixing albumin with a water-miscible solvent, (b) dissolving the Class II or Class IV low solubility molecule in the mixture of albumin and water-miscible solvent to form a solution, (c) adding an agent that prevents self-aggregation of albumin to the mixture of the Class II or Class IV low solubility molecule, water-miscible solvent and albumin, and (d) spray-drying the mixture.

The term ‘solubility’ as described herein includes the meaning of the capacity of a solid chemical substance, called a solute, to dissolve in a liquid solvent. A solute is a substance dissolved in another substance, known as a solvent. A solvent is a substance that dissolves a solute, resulting in a solution.

Specifically, solubility as defined by IUPAC is the analytical composition of a saturated solution expressed as a proportion of a designated solute in a designated solvent. The solubility of a solute generally depends on the physical and chemical properties of the solute and solvent as well as on temperature, pressure and the pH of the solution. The extent of the solubility of a solute in a specific solvent is typically measured as the saturation concentration, where adding more solute does not increase the concentration of the solution and begins to precipitate the excess amount of solute. Typically, this is at a state of equilibrium between the solution and the excess solute (Chavda eta, 2010, Sys Rev Pharm, 1(1):62-69). Therefore, solubility may represent the endpoint in the process of dissolution of a solute, i.e. the maximum amount of the solute that may be dissolved in a solvent (total dissolution capacity), regardless of how fast this occurs. The solubility value of a given solute in a given liquid solvent may therefore be defined as mass (or moles) of solute per volume (or moles) of solvent, e.g. g/L or mg/mL, measured at ambient pressure and temperature. For example, measurement of solubility in water is typically performed at 25° C., pH 7, at ambient pressure. Solubilisation may occur under dynamic equilibrium, which includes the meaning that solubilisation results from the simultaneous and opposing processes of dissolution and phase joining (e.g. precipitation of solids). The solubility equilibrium may occur when the two processes proceed at a constant rate.

The terms ‘dissolution’, ‘dissolving’ and ‘dissolve’ as described herein typically refer to the process by which a solid chemical substance becomes a solute (dissolved component) in a liquid solvent, forming a solution of the solid in the original liquid solvent. In the case of a crystalline solid dissolving in a liquid, the crystalline structure is generally disintegrated such that the separate atoms, ions, or molecules are released. It is understood that full dissolution of a solute in a solvent results in full disintegration of any original structure of the solid (e.g. a crystalline structure). It will also be appreciated that the terms ‘dissolution’, ‘dissolving’ and ‘dissolve’ apply equally when the solid chemical substance consists of a single type of molecule as when the solid chemical substance is a composition or mixture of molecules.

Generally, the Class II or Class IV low solubility molecule is fully dissolved in solution before spray drying. However, it will be appreciated that partial dissolution may be tolerated. For example, the low solubility molecule may be held in a nanodispersed state (nanodispersion) within the solvent. In such a nanodispersion, the drug may not be fully in solution, but may exist as nanosized insoluble material that is stabilised in this form by albumin. Such stabilisation by albumin prevents aggregation of the insoluble material into larger structures. Following spray-drying of such a nanodispersion, the low solubility molecule can much more easily dissolve due to its larger surface area to volume ratio (compared to the ratio before being in a nanodispersion).

The term ‘rate of dissolution’ as described herein typically refers to the rate at which dissolution occurs. For example, the rate of dissolution may be expressed using the Noyes-Whitney equation:

$$\frac{d m}{d T} = A \frac{D}{d} ( {C}_{s} - {C}_{b} )$$

where dm/dt=solute dissolution rate (kg/s); m=mass of dissolved material (kg); t=time (s); A=surface area of the solute particle (m²); D=diffusion coefficient (m/s), which is related in part to the viscosity of the solvent; d=thickness of the concentration gradient (m); Cₛ=particle surface (saturation) concentration (kg or moles/L); C_{b}=concentration in the bulk solvent/solution (kg or moles/L). In another example, the rate of dissolution may include the time taken for dissolution of the maximum amount of solute in the solution according to the solubility of the solute in the solvent at a particular temperature and pressure to occur.

Further description of the terms ‘solubility’, ‘dissolution’ and ‘rate of dissolution’ can be found in Chapter 3 (pages 31-50) of Remington Education: Physical Pharmacy, Blaine T. Smith, Pharmaceutical Press, 2016, (the entire contents of which are incorporated herein by reference).

The term ‘enhancing the solubility and/or the rate of dissolution of a (Class II or Class IV) low solubility molecule’ as described herein includes the meaning that the solubility and/or the rate of dissolution of the molecule is increased after the steps of the method have been carried out on the molecule. Preferably, the solubility and/or the rate of dissolution of the low solubility molecule in an aqueous solvent is enhanced or increased after any one of the methods of the invention is carried out. For example, the solubility and/or the rate of dissolution of the low solubility molecule in water may be increased.

For example, the solubility and/or the rate of dissolution of the molecule may be increased by at least 10% compared to the solubility and/or the rate of dissolution of the molecule prior to the method being carried out, such as by at least 20%, 30%, 40%, 50%, 60%, 70%, 80%, 90%, 100%, 150% or 200%, and preferably by at least 300%, 400%, 500%, 600%, 700%, 800%, 900%, 1000%, 1500%, 2000%, 2500%, 3000%, 4000%, 5000%, 6000%, 7000%, 8000% or 9000%.

Solubility can be determined by any suitable method known in the art. For example, solubility in a designated solvent (e.g. water, phosphate buffer, or a water-miscible solvent) can be determined by dissolving or dispersing the substance in that designated solvent, and measuring the amount of the substance that is in solution, for example by chromatography e.g. high-performance liquid chromatography (HPLC), using standard methods known in the art. Other methods for solubility determination include: generating a pH-solubility profile of test drug in aqueous media with a pH range of 1 to 7.5 using the shake-flask or titration method; analysis by a validated stability-indicating assay; or passing a solution of a drug through a microfilter (e.g. 0.2 micron pore size) and determining the quantity of the solubilised drug by measuring absorbance with a spectrophotometer; or associative neural network tools such as ALOGPS 2.1 (Tetko & Tanchuk, 2002, J Chem Inf Comput Sci, 42:1138-1145; tool available at http://www.vcclab.org/lablalogps/); or determining a partition coefficient using 1-octanol and aqueous solvent (e.g. water), using shake-flask method. Phosphate buffer as described herein includes 1.0 M phosphate buffer, pH 7.4 at 25° C.

It will be appreciated that where performance of any one of the methods of the invention enhances the solubility of a Class II or Class IV low solubility molecule, this includes by increasing the amount of the Class II or Class IV low solubility molecule that can be dissolved at a given temperature and pressure, compared to the amount where the methods of the invention have not been performed.

The rate of dissolution can also be determined by any suitable method known in the art, including measuring dissolution (how much of the solute has dissolved) as a product of time, e.g. after 5, 10, 15, 20 and 30 minutes. In addition to the methods described above for determination of solubility, suitable methods for measuring dissolution include: United States Pharmacopeia (USP) apparatus I (basket) at 100 revolutions per minute (RPM), or USP apparatus II (paddle) at 50 RPM using 900 mL or less (e.g. 500 mL) of any or all of the following dissolution media: 0.1 N HCl or simulated gastric fluid USP without enzymes; pH 4.5 aqueous buffer solution; and pH 6.8 aqueous buffer solution or simulated intestinal fluid USP without enzymes. In particular, the rate of dissolution may be calculated using the Noyes-Whitney equation given above, the individual parameters of which may be determined by any suitable method known in the art.

It will be appreciated that where performance of any one of the methods of the invention enhances the rate of dissolution of a Class II or Class IV low solubility molecule, this includes by increasing the rate of dissolution of the Class II or Class IV low solubility molecule at a given temperature and pressure, compared to the rate of dissolution where the methods of the invention have not been performed. Typically, the rate of dissolution of a Class II or Class IV low solubility molecule would be increased by means of (1) a reduction in the time taken for a given amount of the Class II or Class IV low solubility molecule to dissolve at a given temperature and pressure, compared to the time taken where the methods of the invention have not been performed, and/or by means of (2) an increase in the amount of Class II or Class IV low solubility molecule that has dissolved in a given time at a given temperature and pressure, compared to the amount where the methods of the invention have not been performed.

The term ‘Class II or Class IV low solubility molecule’ as described herein includes the meaning of a Class II or Class IV molecule according to the Biopharmaceutics Classification Scheme (BCS). The Biopharmaceutics Classification Scheme (BCS) was first proposed by Amidon et al (Pharm Res. 1995, 12(3):413-420), following their recognition that drug dissolution and gastrointestinal permeability are the fundamental parameters controlling rate and extent of drug absorption. Biopharmaceutics drugs are assigned to one of four classes in the BCS, defined as: Class I, high solubility, high permeability; Class II, low solubility, high permeability; Class III, high solubility, low permeability; Class IV, low solubility, low permeability. Therefore, Class I and Class III drugs have high solubility and Class II and Class IV drugs have low solubility. Thus, it will be appreciated that the technology of the invention is useful for enhancing the solubility and/or dissolution of both ‘low solubility, high permeability molecules’ and ‘low solubility, low permeability molecules’.

Guidance on the BCS by the United States Food and Drug Administration (FDA) can be found in “Waiver of In Vivo Bioavailability and Bioequivalence Studies for Immediate-Release Solid Oral Dosage Forms Based on a Biopharmaceutics Classification System—Guidance for Industry” (U.S. Department of Health and Human Services, Food and Drug Administration, Center for Drug Evaluation and Research (CDER), December 2017 (Final version), Biopharmaceutics, available at https//www.fda.gov/media/70963/download, the entire contents of which are incorporated herein by reference. Further discussion of the BCS can be found in the review by Chavda et al (Chavda et al, 2010, Sys Rev Pharm, 1(1):62-69), the entire contents of which are incorporated herein by reference.

A molecule such as a drug substance may be considered highly soluble (Class I or Class III) when the highest dose strength is soluble in 250 ml or less of water (or aqueous buffered solution) at 37±1° C. over a pH range of 1 to 8, such as 1 to 8, 1 to 7.5, and/or 1.2 to 6.8. Typically, where solubility is measured empirically, the solubility of a molecule is categorised as being low or high solubility following at least three replicate determinations of solubility in each pH condition tested. A molecule such as a drug substance may be considered to have low solubility (Class II or Class IV) when it does not satisfy the above criterion to be highly soluble. However, measurement of solubility for the purposes of categorising a molecule as being low or high solubility typically takes into consideration whether degradation of the molecule is observed as a function of the buffer composition and/or pH. In such a situation, the method chosen for measuring the concentration of the molecule in order to determine solubility preferably distinguishes the molecule from its degradation products. Additionally in such a situation, the final categorisation of high or low solubility typically factors in any effect on the measurement of solubility that is caused by the degradation products. In this way, a more accurate assessment of the true solubility is possible.

In the context of measurement of solubility, the term aqueous buffered solution as described herein includes a solution comprising water and one or more standard buffers so that it contains a mixture of a weak acid and its conjugate base, or a weak base and its conjugate acid, and is substantially free from proteins, polymers, and macromolecules. For example, standard buffer solutions described in the United States Pharmacopeia, or other suitable aqueous buffer solutions known in the art, may be used.

The term permeability as described herein in relation to determination of classification of Class IV low solubility molecules includes the degree of absorption by the intestines, and therefore may relate to bioavailability. A molecule such as a drug substance may be considered highly permeable (Class I or Class II) when the extent of absorption in humans is determined to be 85% or more, e.g. 90% or more, of an administered dose, for example based on mass-balance or in comparison to an intravenous reference dose. A molecule such as a drug substance may be considered to have low permeability (Class III or Class IV) when it does not satisfy the above criterion to be highly permeable.

It will be appreciated that permeability can be determined using any suitable method known in the art. For example, methods for permeability determination include determining the extent of absorption in humans by mass-balance pharmacokinetic studies and/or absolute bioavailability studies. Additional methods for permeability determination are intestinal permeability methods including in vivo intestinal perfusion studies in humans, in vivo or in situ intestinal perfusion studies in animals, in vitro permeation experiments with excised human or animal intestinal tissue, and in vitro permeation experiments across epithelial cell monolayers, e.g. Caco-2 cells or TC-7 cells. Mass-balance studies may use unlabelled, stable isotopes or radiolabelled drug substances to determine the extent of drug absorption. In case of conflicting information from different types of studies, human data generally supersede in vitro or animal data. In vivo or in situ animal models and in vitro methods, such as those using cultured monolayers of animal or human epithelial cells, may be considered appropriate for assessing the permeability of passively transported molecules.

The observed low permeability of some drug substances in humans could be caused by efflux of drugs via membrane efflux transporters such as P-glycoprotein. When the efflux transporters are absent in such models, or their degree of expression is low compared to that in humans, there may be a greater likelihood of misclassification of permeability class for a molecule subject to efflux compared to a molecule transported passively. Therefore, expression of known transporters in selected study systems should typically be characterised, e.g. using bidirectional transport studies using selected model drugs or chemicals at concentrations that do not saturate the efflux system (e.g. digoxin, vinblastine, rhodamine 123). It may be possible to assume an apparent passive transport mechanism when one of the following conditions is satisfied:
1. a linear (pharmacokinetic) relationship between the dose (e.g. a relevant clinical dose range) and measures of bioavailability (e.g. area under the concentration-type curve) is demonstrated in humans;
2. lack of dependence of the measured in vivo or in situ permeability is demonstrated in an animal model on initial molecule concentration (e.g. 0.01, 0.1 and 1 times the highest strength dissolved in 250 ml) in the perfusion fluid;
3. lack of dependence of the measured in vitro permeability on initial molecule concentration (e.g. 0.01, 0.1 and 1 times the highest strength dissolved in 250 ml) is demonstrated, or on transport direction (e.g. no statistically significant difference in the rate of transport between the apical-to-basolateral and basolateral-to-apical direction for the molecule concentrations selected) using a suitable in vitro cell culture method that has been shown to express known efflux transporters (e.g. P-glycoprotein).

The term bioavailability as described herein includes the measurement of the rate and extent to which a drug reaches the site of action. It includes the meaning of the fraction of an administered dose of unchanged drug that reaches the systemic circulation (one of the principal pharmacokinetic properties of drugs). Absolute bioavailability includes the comparison of the amount of the active drug in systemic circulation following non-intravenous administration (i.e. after oral, ocular, rectal, transdermal, subcutaneous, or sublingual administration), with the amount of the same drug following intravenous administration and is typically taken as a ratio of areas under the curves of a plasma drug concentration vs time plot after both intravenous and non-intravenous administration. By definition, when a medication is administered intravenously, its bioavailability is 100%.

It will be appreciated that the BCS classification has already been determined for many compounds and is available in standard chemical databases. In addition, BCS classification may be obtained from online databases such as https://mypharmatools.com/databases/dissolution, and additional BCS classification sources are well known in the art, for example as reviewed in Dahan et al The AAPS Journal, Vol. 11, No. 4, December 2009.

The term ‘low solubility molecule’ as described herein includes the meaning of a Class II or Class IV low solubility molecule as described above. In some embodiments, the low solubility molecule is a Class II molecule. In other embodiments, the low solubility molecule is a Class IV molecule. The term ‘molecule’ in this context includes, but is not limited to, drugs and active pharmaceutical ingredients (APIs).

The low solubility molecule may have a solubility in water of less than or equal to 10 mg/mL, 8 mg/mL, 6 mg/mL, 5 mg/mL, 4 mg/mL, 2 mg/mL, 1.5 mg/mL, 1 mg/mL, 0.8 mg/mL, 0.6 mg/mL, 0.4 mg/mL, 0.2 mg/mL, 0.1 mg/mL, 0.05 mg/mL, 0.02 mg/mL, 0.01 mg/mL, 0.005 mg/mL, 0.001 mg/mL, 0.0005 mg/mL or 0.0001 mg/mL.

The low solubility molecule may have a solubility in water of greater than or equal to 0.00001 mg/mL, 0.0001 mg/mL, 0.0005 mg/mL, 0.001 mg/mL, 0.005 mg/mL, 0.01 mg/mL, 0.02 mg/mL, 0.05 mg/mL, 0.1 mg/mL, 0.2 mg/mL, 0.4 mg/mL, 0.6 mg/mL, 0.8 mg/mL, 1 mg/mL, 1.5 mg/mL, 2 mg/mL, 4 mg/mL, 5 mg/mL, 6 mg/mL or 8 mg/mL.

Preferably, the low solubility molecule has a solubility in water of less than or equal to 10 mg/mL.

It will be appreciated that the solubility of the low solubility molecule may be higher or lower in aqueous solvents other than water, e.g. phosphate buffer or phosphate buffered saline, than the solubility in water itself.

The low solubility molecule may have a solubility in phosphate buffer of less than or equal to 10 mg/mL. It will be appreciated that the low solubility molecule may have a solubility of less than or equal to 10 mg/mL in other aqueous solutions that are suitable for intravenous administration. Such solutions are well known in the art and include 5% dextrose in water (D5W solution), and isotonic normal saline solution (0.9% NaCl).

It will be appreciated that the Class II or Class IV low solubility molecule the solubility of which is enhanced by the methods described herein must be soluble, i.e. fully or partially dissolvable, in the water-miscible solvent. Preferably, the low solubility molecule has a solubility in the water-miscible solvent(s) of at least 1 mg/mL, for example at least 1.2 mg/mL, 1.4 mg/mL, 1.6 mg/mL, 1.8 mg/mL, 2.0 mg/mL, 2.2 mg/mL, 2.4 mg/mL, 2.6 mg/mL, 2.8 mg/mL or 3.0 mg/mL. For example, when the water-miscible solvent is ethanol, it is preferred that the low solubility molecule has a solubility in ethanol of at least 1 mg/mL, for example at least 1.2 mg/mL, 1.4 mg/mL, 1.6 mg/mL, 1.8 mg/mL, 2.0 mg/mL, 2.2 mg/mL, 2.4 mg/mL, 2.6 mg/mL, 2.8 mg/mL or 3.0 mg/mL. Methods for assessing solubility in a water-miscible solvent are known in the art and include those described above.

It will be appreciated that if the low solubility molecule has a poor solubility in any given water-miscible solvent(s), another water-miscible solvent(s) may be used. It will also be appreciated that a low solubility molecule that is only partially soluble in a designated water-miscible solvent(s) may still be suitable for use in the methods of the invention because the existence of a partial solution allows the low solubility molecule to be mixed into the albumin prior to spray drying. A larger volume of the water-miscible solvent may also be required in order to dissolve the desired amount of the low solubility molecule. Nevertheless, solubility and dispersability may be further increased by adding one or more surfactants and/or dispersants.

The low solubility molecule may have a physiological charge of +1, 0 or −1. The term ‘physiological charge’ as described herein includes the meaning of the standard chemical definition of the total charge of the molecule, namely the sum of charges of all charged groups, e.g. carboxyl anion (−1), protonated amine (+1), when measured at pH 7.4. The charge of each potentially charged group can be calculated at a given pH by reference to the pKa for each potentially charged group, according to methods well known in the art. The pKa for each potentially charged group may also be determined according to methods well known in the art.

The low solubility molecule may include one or more rings. The ring(s) can be saturated or unsaturated. The ring(s) can be homocyclic or heterocylic. The term ‘rings’ as described herein may therefore include aromatic (unsaturated) hydrocarbon rings, saturated hydrocarbon rings, saturated homocyclic rings, unsaturated heterocyclic rings and saturated heterocyclic rings. The ring(s) may have between 3 and 7 molecules that form the ring itself. Such rings may also contain a variety of chemical functionalities including hydrogen, hydroxyl groups (—OH), ketones and aldehydes, various halogens, various sulphur containing groups, alkyl chains, and ester chains. Heterocyclic rings may contain, among other atoms, carbon, nitrogen, oxygen, sulphur and phosphorous, and the rings may contain saturated as well as unsaturated bonds.

In one embodiment of the first aspect of the invention, the low solubility molecule has a solubility in water of less than or equal to 0.02 mg/ml, a physiological charge of between +1 and −1, and greater than or equal to 4 rings.

The low solubility molecule may have a molecular weight of less than or equal to 3000 g/mol, 2500 g/mol, 2000 g/mol, 1750 g/mol, 1500 g/mol, 1250 g/mol, 1000 g/mol, 900 g/mol, 800 g/mol, 700 g/mol, 600 g/mol, 500 g/mol, 450 g/mol, 400 g/mol, 350 g/mol, 300 g/mol, 250 g/mol, 200 g/mol or 150 g/mol, and/or a molecular weight of greater than or equal to 150 g/mol, 200 g/mol, 250 g/mol, 300 g/mol, 350 g/mol, 400 g/mol, 450 g/mol, 500 g/mol, 600 g/mol, 700 g/mol, 800 g/mol, 900 g/mol, 1000 g/mol, 1250 g/mol, 1500 g/mol, 1750 g/mol, 2000 g/mol, or 2500 g/mol. For example, the low solubility molecule may have a molecular weight of 150 g/mol to 3000 g/mol.

The low solubility molecule as described herein may be a peptide, a small molecule, a nucleic acid, a peptide nucleic acid, a carbohydrate, or a natural product. The term ‘peptide’ as described herein includes dipeptides, tripeptides, tetrapeptides, oligopeptides, polypeptides, natural peptides, synthetic peptides, cyclic peptides and peptides using D-amino acids. The term ‘small molecule’ as described herein includes low molecular weight (<900 daltons) organic compounds that may help regulate a biological process, with a size on the order of 1 nm, and also organic compounds <500 daltons. The term ‘nucleic acid’ as described herein includes DNA, RNA, DNA-RNA hybrids, mixed DNA-RNA polymers, locked nucleic acids, morpholinos, ribozymes, small hairpin RNA, small interfering RNA, guide RNA, xeno nucleic acids (XNA), XNAzymes, and other nucleic acid analogues. The term ‘peptide nucleic acid’ as described herein includes any molecule comprising both one or more amino acids and one or more nucleic acid bases, and also includes uncharged nucleic acids (UNA) and polyamide nucleic acids, e.g. those described in Nielsen et al (Science 1991, 254(5037):1497-1500). The term ‘carbohydrate’ as described herein includes oligosaccharides and polysaccharides, as well as hydrophobic modified monosaccharides, disaccharides etc. Hence, the term ‘carbohydrate’ as described herein includes all sugar polymers. The term ‘natural product’ as described herein includes fungal metabolites, fermentation broth products, tacrolimus, paclitaxel etc.

Examples of Class II low solubility molecules and Class IV low solubility molecules include those provided in Table A below. Any of the molecules listed therein may be used in the methods of the present invention.

TABLE A
Examples of low solubility molecules
Class II compounds

Aceclofenac, Albendazole, Atorvastatin, Azithromycin, Benidipine, Bicalutamide,
Bisacodyl, Cabergoline, Candesartan cilexetil, Carbamazepine, Carvedilol, Celecoxib,
Chloroquine, Chlorpromazine, Cilostazol, Clarithromycin, Clofazimine, Clopidogrel,
Cyclosporine, Cyproterone, Diazepam, Diclofenac, Diloxanide, Ebastine, Efavirenz,
Epalrestat, Eprosartan, Erythromycin, Ethyl icosapentate, Ezetimibe, Fenofibrate,
Flurbiprofen, Gefitinib, Glimepiride, Glipizide, Glyburide (glibenclamide), Griseofulvin,
Haloperidol, Hydroxyzine, Ibuprofen, Imatinib, Indinavir, Iopanoic acid, Irbesartan,
Isotretinoin, Itraconazole, Ivermectin, Ketoprofen, Lamotrigine, Levodopa, Lopinavir,
Loratadine, Lorazepam, Lovastatin, Manidipine, Mebendazole, Medroxyprogesterone,
Meloxicam, Menatetrenone, Metaxalone, Metoclopramide, Mosapride, Mycophenolate
mofetil, Nabumetone, Nelfinavir, Nevirapine, Nicergoline, Niclosamide, Nifedipine,
Nilvadipine, Nimesulide, Orlistat, Phenytoin, Pioglitazone, Pranlukast, Praziquantel,
Pyrantel, Pyrimethamine, Quetiapine, Quinine, Raloxifene, Rapamycin (sirolimus),
Rebamipide, Retinol, Rifampicin, Risperidone, Ritonavir, Rofecoxib, Simvastatin,
Spironolactone, Sulfasalazine, Tacrolimus, Tamoxifen, Telmisartan, Teprenone,
Ticlopidine, Tocopherol nicotinate, Triflusal, Ursodeoxycholic Acid, Valproic acid,
Valsartan, Verapamil, Warfarin, Zaltoprofen

Class IV compounds

Acetaminophen (Paracetamol), Acetazolamide, Acetylsalicylic acid, Acyclovir,
Allopurinol, Aluminium hydroxide, Amoxicillin, Azathioprine, Cefdinir, Cefixime,
Cefotiam, Cefpodoxime, Cefuroxime axetil, Dapsone, Doxycycline, Famotidine, Folic
acid, Furosemide, Hydrochlorothiazide, L-carbocysteine, Linezolid, Mesalamine,
Metronidazole, Modafinil, Nalidixic acid, Nitrofurantoin, Nystatin, Olanzapine,
Oxcarbazepine, Oxycodone, Phenobarbital, Propylthiouracil, Sulfadiazine,
Sulfamethoxazole, Sulpiride, Theophylline, Trimethoprim

Compounds that are empirically Class II compounds but are Class IV compounds
according to in silico cLogP determination

Cefditoren, Gliclazide, Tosufloxacin

Compounds that are empirically Class IV compounds but are Class II compounds
according to in silica cLogP determination

Dexamethasone, Methylphenidate, Roxithromycin, Sultamicillin

Compounds with low solubility but for which permeability
has not yet been determined

Sennoside A

Preferably, the low solubility molecule as described herein is a Class II compound selected from the group: aceclofenac, albendazole, atovaquone, bicalutamide, clozapine, danazol, ezetimibe, fenofibrate, glibenclamide, itraconazole, lopinavir, modafinil, nabilone, nimesulide, nimodipine, paliperidone, phenytoin, propofol, prostaglandin E1, rapamycin, repaglinide, risperidone, ritonavir, tacrolimus, teniposide, tretinoin, valsartan, vincristine, voriconazole, zipradisone.

Preferably, the low solubility molecule as described herein is a Class IV compound selected from the group: acyclovir, allopurinol, amoxicillin, amphotericin b, aripiprazole, bifonazole, carflizomib, cefuroxime axetil, docetaxel, etravirine, linezolid, oxcarbazepine, paclitaxel, rimiducid.

Most preferably, the low solubility molecule as described herein is a Class II compound selected from the group: danazol, ezetimibe, lopinavir, phenytoin, rapamycin, ritonavir and tacrolimus; or a Class IV compound selected from the group: bifonazole, etravirine, paclitaxel and rimiducid. Characterising parameters of these low solubility molecules are presented in Table B, and it will be appreciated that the methods of the invention will be applicable to molecules having similar properties.

TABLE B

| | | Water | | Number | Molecular | | |
|---|---|---|---|---|---|---|---|
| | BCS | Solubility | Physiological | of | Weight | PubChem | DrugBank |
| Name | Classification | (mg/mL) | Charge | Rings | (g/mol) | ID | ID |
| Bifonazole | Class IV | 0.00245 | 0 | 4 | 310.4 | 2378 | DB04794 |
| Danazol | Class II | 0.0176 | 0 | 5 | 337.463 | 28417 | DB01406 |
| Etravirine | Class IV | 0.0169 | 0 | 3 | 435.285 | 193962 | DB06414 |
| Ezetimibe | Class II | 0.00846 | 0 | 4 | 409.433 | 150311 | DB00973 |
| Lopinavir | Class II | 0.00192 | 0 | 4 | 628.814 | 92727 | DB01601 |
| Paclitaxel | Class IV | 0.00556 | 0 | 7 | 853.918 | 36314 | DB01229 |
| Phenytoin | Class II | 0.0711 | 0 | 3 | 252.273 | 1775 | DB00252 |
| Rapamycin | Class II | 0.00173 | 0 | 4 | 914.2 | 5284616 | DB00877 |
| (sirolimus) | | | | | | | |
| Rimiducid | Class IV | 0.000001 | 0 | 8 | 1411.65 | 16135625; | DB04974 |
| (AP1903) | | | | | | 126970623 | |
| Ritonavir | Class II | 0.00126 | 0 | 4 | 720.948 | 392622 | DB00503 |
| Tacrolimus | Class II | 0.00402 | 0 | 4 | 804.031 | 445643 | DB00864 |

The molecular weights in Table B were obtained from PubChem, at the following address: https//pubchem.ncbi.nlm.nih.gov/compound/<PubChem ID>. The values for water solubility, physiological charge and number of rings in Table B were obtained from DrugBank, at the following address: https://www.drugbank.ca/drugs/<DrugBank ID>, except for the water solubility of rimiducid which was provided by Bellicum Pharmaceuticals (estimated at 700 pM, i.e. approximately 0.000001 mg/mL).

In an embodiment, the low solubility molecule is a Class IV molecule having a solubility in water less than 0.005 mg/mL, a physiological charge of 0, a number of rings greater than or equal to 4, and a molecular weight of less than 350 g/mol, preferably bifonazole.

In an embodiment, the low solubility molecule is a Class II molecule having a solubility in water less than 0.05 mg/mL, a physiological charge of 0, a number of rings greater than or equal to 5, and a molecular weight of less than 350 g/mol, preferably danazol.

In an embodiment, the low solubility molecule is a Class IV molecule having a solubility in water less than 0.05 mg/mL, a physiological charge of 0, a number of rings greater than or equal to 3, and a molecular weight of less than 450 g/mol, preferably etravirine.

In an embodiment, the low solubility molecule is a Class II molecule having a solubility in water less than 0.01 mg/mL, a physiological charge of 0, a number of rings greater than or equal to 2, and a molecular weight of less than 450 g/mol, preferably ezetimibe.

In an embodiment, the low solubility molecule is a Class II molecule having a solubility in water less than 0.005 mg/mL, a physiological charge of 0, a number of rings greater than or equal to 4, and a molecular weight of less than 650 g/mol, preferably lopinavir.

In an embodiment, the low solubility molecule is a Class II molecule having a solubility in water less than 0.1 mg/mL, a physiological charge of 0, a number of rings greater than or equal to 3, and a molecular weight of less than 300 g/mol, preferably phenytoin.

In an embodiment, the low solubility molecule is a Class IV molecule having a solubility in water less than 0.000002 mg/mL, a physiological charge of 0, a number of rings greater than or equal to 8, and a molecular weight of less than 1500 g/mol, preferably rimiducid (CAS 195514-63-7). The synthesis of rimiducid (also known as AP1903) is described in Clackson et al (1998) Proc Natl Acad Sci USA 95(18):10437-4, and its IUPAC name is 1,2-Ethanediylbis[imino(2-oxo-2,1-ethanediyl)oxy-3,1-phenylene(1R)-3-(3,4-dimethoxyphen-yl)-1,1-propanediyl] (2S,2'S)bis{1-[(2S)-2-(3,4,5-trimethoxyphenyl)butanoyl]-2-piperidine-carboxylate}.

In an embodiment, the low solubility molecule is a Class II molecule having a solubility in water less than 0.002 mg/mL, a physiological charge of 0, a number of rings greater than or equal to 4, and a molecular weight of less than 950 g/mol, preferably rapamycin (also known as sirolimus).

In an embodiment, the low solubility molecule is a Class II molecule having a solubility in water less than 0.005 mg/mL, a physiological charge of 0, a number of rings greater than or equal to 4, and a molecular weight of less than 750 g/mol, preferably ritonavir.

In an embodiment, the low solubility molecule is a Class II molecule having a solubility in water less than 0.005 mg/mL, a physiological charge of 0, a number of rings greater than or equal to 4, and a molecular weight of less than 850 g/mol, preferably tacrolimus.

In an embodiment, the low solubility molecule is a Class IV molecule having a solubility in water less than 0.01 mg/mL, a physiological charge of 0, a number of rings greater than or equal to 7, and a molecular weight of less than 900 g/mol, preferably paclitaxel.

The term ‘miscible’ as described herein includes the meaning of the property of liquids to mix in all proportions (that is, to fully dissolve in each other at any concentration), forming a homogeneous solution.

Therefore, the term ‘water-miscible solvent’ as described herein includes the meaning of a solvent, other than water, that can mix completely with water to form a homogeneous solution. The water-miscible solvent may be a non-aqueous solvent that is nonetheless miscible with water. It will be appreciated that the term ‘water-miscible solvent’ also includes a mixture of water-miscible solvents (preferably where they are also miscible with each other), e.g. ethanol/DMSO, and also includes where water may form one component of the mixture, e.g. 70% ethanol/30% water.

In the methods described herein, the water-miscible solvent may comprise one or more water-miscible solvents. When only one water-miscible solvent is used in the methods described herein, this cannot be water. Preferably, the water-miscible solvent(s) in the methods described herein is a solvent(s) with low toxic potential that may therefore be considered suitable for pharmaceutical use, for example any of the following FDA Class 3 solvents, which by definition are solvents with a permitted daily exposure of 50 mg or more per day: acetic acid, acetone, dimethylsulphoxide (DMSO), ethanol, formic acid (methanoic acid), 1-propanol, 2-propanol (isopropanol), and tetrahydrofuran (oxolane) (see US FDA, “Guidance for Industry—‘Impurities: Residual Solvents’, VICH GL18”, 18-20 May 1999, and US FDA, “Guidance for Industry—‘Impurities: Residual Solvents in New Veterinary Medicinal Products, Active Substances and Excipients (Revision)’, VICH GL18(R)”, 2 Nov. 2011 https-/www.fda.gov/media/70410/download). Another example of a water-miscible solvent is N,N-dimethylformamide (DMF). A preferred example of a water-miscible solvent is ethanol.

The water-miscible solvent(s) in the methods described herein (e.g. ethanol) may comprise water, for example the water may be present at not more than 95%, 90%, 85%, 80%, 75%, 70%, 65%, 60%, 55%, 50%, 45%, 40%, 35%, 30%, 25%, 20%, 15%, 10%, 5%, 2%, 1% of the mixture of the water-miscible solvent (e.g. ethanol) and water. This is particularly desirable when the low solubility molecule together with albumin are simultaneously added to the water-miscible solvent(s).

The term ‘albumin’ as described herein includes the meaning of a protein having the same and/or very similar tertiary structure as human serum albumin (HSA; in particular SEQ ID NO:1; or UniProt sequence P02768-1, accessed 8 May 2019) or HSA domains and has similar properties of HSA or the relevant domains. Similar tertiary structures are, for example, the structures of the albumins from species other than human, e.g. non-human primate albumin, (such as chimpanzee albumin (e.g. predicted sequence GenBank XP_517233.2), gorilla albumin or macaque albumin (e.g. GenBank NP_001182578)), rodent albumin (such as hamster albumin (e.g. GenBank A6YF56), guinea pig albumin (e.g. UniProt Q6WDN9-1), mouse albumin (e.g. GenBank AAH49971 or UniProt P07724-1 Version 3) and rat albumin (e.g. GenBank AAH85359 or UniProt P02770-1 Version 2)), bovine albumin (such as cow albumin (e.g. UniProt P02769-1)), equine albumin (such as horse albumin (e.g. UniProt P35747-1) ordonkey albumin (e.g. UniProt Q5XLE4-1)), rabbit albumin (e.g. UniProt P49065-1 Version 2), goat albumin (e.g. GenBank ACF10391), sheep albumin (e.g. UniProt P14639-1), dog albumin (e.g. UniProt P49822-1), chicken albumin (e.g. UniProt P19121-1 Version 2) and pig albumin (e.g. UniProt P08835-1 Version 2), or any one of SEQ ID NOs:4 to 19 of WO 2013/006675, which are incorporated herein by reference. All of these albumins are included in the scope of the present invention. Mature forms of albumin (e.g. forms where all post translational modifications and/or processing steps have been completed) are particularly preferred, and the skilled person is able to identify mature forms using publicly available information such as protein databanks and/or by using signal peptide recognition software such as SignalP (e.g., SignalP (Nielsen et al, 1997, Protein Engineering 10(1):1-6)). SignalP Version 4.0 is preferred (Petersen et al, 2011, Nat Methods 8(10):785-786). An albumin preparation for use in the methods and compositions of the present invention may comprise one or more (several) albumins.

Some of the major properties of albumin are 1) its ability to regulate plasma volume, ii) a long plasma half-life of around 19 days±5 days, iii) ligand binding, e.g. binding of endogenous molecules such as acidic, lipophilic compounds including bilirubin fatty acids, hemin and thyroxine (see also Table 1 of Kragh-Hansen et al, 2002, Biol Pharm Bull 25(6):695-704, hereby incorporated by reference), iv) binding of small organic compounds with acidic or electronegative features, e.g. drugs such as warfarin, diazepam, ibuprofen and paclitaxel (see also 1 of Kragh-Hansen et al, 2002, Biol Pharm Bull 25(6):695-704, hereby incorporated by reference). Not all of these properties need to be fulfilled in order to characterise a protein or fragment as an albumin. If a fragment, for example, does not comprise a domain responsible for binding certain ligands or organic compounds, the variant of such a fragment is not be expected to have these properties either.

The term ‘albumin’ includes variants, and/or derivatives such as fusions and/or conjugations of an albumin or of an albumin variant.

By ‘variant’ we include the meaning of a polypeptide derived from a parent albumin comprising an alteration, i.e. a substitution, insertion and/or deletion, at one or more (several) positions. A substitution includes the meaning of a replacement of an amino acid occupying a position with a different amino acid; a deletion includes the meaning of the removal of an amino acid occupying a position; and an insertion includes the meaning of adding amino acids (e.g. 1-3 amino acids) adjacent to an amino acid occupying a position. For example, the altered polypeptide (variant) can be obtained through human intervention by modification of the polynucleotide sequence encoding the parent albumin. The variant albumin is preferably at least 70%, preferably at least 75%, more preferably at least 80%, more preferably at least 85%, even more preferably at least 90%, most preferably at least 95%, at least 96%, at least 97%, at least 98%, at least 99%, or 100% identical to SEQ ID NO: 1:

wild-type human albumin
SEQ ID NO: 1
DAHKSEVAHRFKDLGEENFKALVLIAFAQYLQQCPFEDHVKLVNEVTE
FAKTCVADESAENCDKSLHTLFGDKLCTVATLRETYGEMADCCAKQEP
ERNECFLQHKDDNPNLPRLVRPEVDVMCTAFHDNEETFLKKYLYEIAR
RHPYFYAPELLFFAKRYKAAFTECCQAADKAACLLPKLDELRDEGKAS
SAKQRLKCASLQKFGERAFKAWAVARLSQRFPKAEFAEVSKLVTDLTK
VHTECCHGDLLECADDRADLAKYICENQDSISSKLKECCEKPLLEKSH
CIAEVENDEMPADLPSLAADFVESKDVCKNYAEAKDVFLGMFLYEYAR
RHPDYSVVLLLRLAKTYETTLEKCCAAADPHECYAKVFDEFKPLVEEP
QNLIKQNCELFEQLGEYKFQNALLVRYTKKVPQVSTPTLVEVSRNLGK
VGSKCCKHPEAKRMPCAEDYLSVVLNQLCVLHEKTPVSDRVTKCCTES
LVNRRPCFSALEVDETYVPKEFNAETFTFHADICTLSEKERQIKKQTA
LVELVKHKPKATKEQLKAVMDDFAAFVEKCCKADDKETCFAEEGKKLV
AASQAALGL

Typically, the variant albumin maintains at least one of the major properties of the parent albumin or a similar tertiary structure as HSA. For the purposes of the present invention, the sequence identity between two amino acid sequences may be determined using the Needleman-Wunsch algorithm (Needleman & Wunsch, 1970, J Mol Biol 48(3):443-453) as implemented in the Needle program of the EMBOSS package (EMBOSS: The European Molecular Biology Open Software Suite, Rice et al, 2000, Trends Genet 16(6):276-277), preferably version 5.0.0 or later. The typical parameters used are gap open penalty of 10, gap extension penalty of 0.5, and the EBLOSUM62 (EMBOSS version of BLOSUM62) substitution matrix. The output of Needle labelled “longest identity” (obtained using the −nobrief option) may be used as the percent identity and may be calculated as follows: (Identical Residues×100)/(Length of Alignment −Total Number of Gaps in Alignment).

The variant may possess altered binding affinity to FcRn and/or an altered rate of transcytosis across endothelia, epithelia and/or mesothelia mono cell-layer when compared to the parent albumin. The variant polypeptide sequence is preferably one which is not found in nature. A variant includes a fragment, e.g. comprising or consisting of at least 100, 150, 200, 250, 300, 350, 400, 450, 500, 550 contiguous amino acids of an albumin.

Examples of albumin variants include those described in WO 2011/051489, WO 2011/124718, WO 2012/059486, WO 2012/150319, WO 2014/072481, WO 2013/135896, WO 2015/036579, WO 2010/092135, WO 2013/075066, WO 2014/179657, WO 2009/126920, WO 2010/059315, WO 2011/103076, WO 2012/112188, WO 2015/063611, and WO 2017/029407 (the contents of which are incorporated herein by reference in their entirety).

The term ‘wild-type’ (WT) albumin as described herein includes the meaning of an albumin having the same amino acid sequence as the predominant allelic variant of albumins naturally found in an animal or in a human being. SEQ ID NO: 1 is an example of a wild-type albumin, being wild-type albumin from Homo sapiens.

The term ‘parent’ or ‘parent albumin’ as described herein includes the meaning of an albumin to which an alteration is made to produce the albumin variants which may be used in the methods and compositions of the present invention. The parent may be a naturally occurring (wild-type) polypeptide or an allele thereof or a variant thereof such as a variant described in WO 2011/051489 or a variant or derivative described in WO 2011/124718.

The term ‘derivative’ as described herein includes fusions and/or conjugations of an albumin or of an albumin variant.

By ‘fusion’ we include the meaning of a genetic fusion of albumin (or a variant or fragment thereof) and a non-albumin protein. The non-albumin protein may be a therapeutic, prophylactic, or diagnostic protein. Examples of albumin fusions are provided in EP 624195, WO 01/79271, WO 03/059934, WO 03/060071, WO 2011/051489, WO 2011/124718, WO 2012/059486, WO 2012/150319, WO 2014/072481, WO 2013/135896, WO 2015/036579, WO 2010/092135, and WO 2017/029407 (the contents of which are incorporated herein by reference in their entirety).

By ‘conjugation’ we include the meaning of an albumin (or a variant or fragment or fusion thereof) to which a non-albumin moiety is chemically conjugated. The non-albumin moiety may be a therapeutic, prophylactic, or diagnostic protein. Examples of albumin conjugations are provided in WO 2011/124718 and WO 2011/051489 (incorporated herein by reference in their entirety).

Preferably, the albumin used in the methods and compositions of the present invention is recombinant albumin, or human albumin, and most preferably recombinant human albumin. In some embodiments, the recombinant human albumin may be a fusion, variant or derivative. Preferably, human albumin is wild-type human albumin with an amino acid sequence according to SEQ ID NO: 1. Preferably, recombinant human albumin as described herein also has an amino acid sequence according to SEQ ID NO: 1.

The term ‘recombinant albumin’ means that the albumin may be sourced from a recombinant organism such as a recombinant microorganism, recombinant plant or recombinant animal. Since some users prefer animal-free ingredients, it is more preferred that the albumin is sourced from a non-animal recombinant source, such as a recombinant microorganism or recombinant plant. Preferred microorganisms include prokaryotes and, more preferably, eukaryotes such as animals, plants, fungi or yeasts, for example, but not limited to, the following species in which albumins have been successfully expressed as recombinant proteins:
fungi, including but not limited to Aspergillus (WO 2006/066595), Kluyveromyces (Fleer et al, Bio/Technology, 1991, 9(10):968-975). Pichia (Kobayashi et al, Ther Apher, 1998, 2(4):257-262) and Saccharomyces (Sleep et al, Bio/technology, 1990, 8(1):42-46);
bacteria (Pandjaitan et al, J Allergy Clin Immunol, 2000, 105(2 Pt 1):279-285), including Bacillus subtilis (Saunders et al, J Bacteriol, 1987, 169(7):2917-2925);
animals (Barash et al, Transgenic Res, 1993, 2(5):266-276)
plants, including but not limited to potato, tobacco (Sijmons et al, Bio/technology, 1990, 8(3):217-221; Farran et al, Transgenic Res, 2002, 11(4):337-346) and rice e.g. Oryza sativa);
mammalian cells such as CHO and HEK.

It will be appreciated that it is preferable for the albumin for use in the methods of the invention to be provided in the form of an albumin preparation, which may include one or more excipients. It is preferable for such albumin preparations to be in aqueous solution.

Particularly preferred forms of (recombinant human) albumin preparations for use in accordance with the methods and compositions of the present invention include those manufactured in yeast, particularly in Saccharomyces cerevisiae such as the following known commercial presentations of recombinant yeast-derived albumin: Recombumin® Alpha (formerly Albucult®), Recombumin® Prime (formerly Recombumin®) and/or Recombumin® Elite (formerly AlbIX®) (all sourced from Albumedix Limited); or any preparation that is similar thereto.

Suitable methods for the production of exemplary recombinant yeast-derived albumin preparations, and such preparations per se, are described in WO 2000/044772 and/or WO 2013/006675, the contents of which are incorporated herein by reference in their entirety. EP 1329460 (A1), EP 1329461 (A1), EP 1329462 (A1) and EP 1710250 (A1) (the contents of each of which are incorporated herein by reference in their entirety) also describe methods suitable for the production of recombinant yeast-derived albumin preparations, and such preparations per se, which may be potentially suitable for use in the methods and compositions of the present invention.

For example, the albumin preparation may comprise:
from 25 to 400 g/L albumin, preferably from 50 to 400 g/L albumin, and wherein preferably the albumin is recombinant albumin;
a solvent;
from 200 mM to 1000 mM cations, preferably from 200 to 350 mM cations, and wherein preferably the cations are selected from sodium, potassium, calcium, magnesium and ammonium, most preferably sodium ions;
less than or equal to 5 mM octanoate, preferably less than or equal to 1 mM octanoate;
(a) less than 5 mM amino acids (such as N-acetyl tryptophan), preferably less than 1 mM amino acids, most preferably being substantially free of amino acids, and/or (b) less than 20 mg/L detergent (such as polysorbate 80), preferably less than 5 mg/L detergent, most preferably being substantially free of detergent;

and having a pH from about 5.0 to about 8.0, preferably from about pH 6.0 to about pH 7.0, most preferably about pH 6.5.

For example, the albumin preparation may comprise: 50 to 250 g/L albumin, 225 to 275 mM Na⁺; 20 to 30 mM phosphate; less than 2 mM octanoate, preferably being substantially free of octanoate; and having a pH of about 6.5; such as Recombumin® Elite (sourced from Albumedix Limited).

The albumin used in the methods described herein may exhibit one or more of the following properties:
(a) less than 0.5% (w/w) binds to Concanavalin A, preferably less than 0.4%, 0.3%, 0.2% or 0.15%; and/or
(b) a glycation level of less than 0.6 moles hexose/mole of protein, and preferably less than 0.10, 0.075 or 0.05 moles hexose/mole of protein.

Preferably, the albumin used in the methods described herein:
(a) is at least about 95%, 96%, 97%, 98%, more preferably at least about 99.5% monomeric and dimeric, preferably essentially 100% monomeric and dimeric (as used in this context, the term “about”, can include meaning oft 1%, 0.5%, 0.4%, 0.3%, 0.3%, 0.1% or less);
(b) is at least about 93%, 94%, 95%, 96% or 97% monomeric (as used in this context, the term “about”, can include meaning of ±1%, 0.5%, 0.4%, 0.3%, 0.3%, 0.1% or less); and/or
(c) has an albumin polymer content of not greater, and preferably less, than about 1.0% (w/w) 0.1% (w/w) or 0.01% (w/w). As used in this context, the term “about”, can include meaning of ±50%, 40%, 30%, 20%, 10%, 5% 1%, 0.5%, 0.4%, 0.3%, 0.3%, 0.1% or less of the stated value; e.g. 1.0% (w/v)±50% is the range of 0.5 to 1.5% (w/v). As used in this context, the term “polymer” as applied to albumin is distinct from monomeric and dimeric forms.

The albumin preparation used in the methods described herein, may comprise, consist essentially of, or consist of, albumin protein, cations (such as sodium, potassium, calcium, magnesium, ammonium, preferably sodium) and balancing anions (such as chloride, phosphate, sulfate, citrate or acetate, preferably chloride or phosphate), water, and optionally octanoate and polysorbate 80.

The albumin preparation used in the methods described herein, may comprise octanoate at less than 35 mM, 32.5 mM, 30 mM, 28 mM, 26 mM, 24 mM, 22 mM, 20 mM, 18 mM, 16 mM, 15 mM, 14 mM, 12 mM, 10 mM, 8 mM, 6 mM, 5 mM, 4 mM, 3 mM, 2 mM, 1 mM, 0.5 mM, 0.4 mM, 0.3 mM, 0.2 mM, 0.1 mM, 0.01 mM, 0.001 mM, may be substantially free of octanoate, or may be free of octanoate.

The albumin preparation used in the methods described herein, may have an overall fatty acid content less than or equal to 35 mM, 32.5 mM, 30 mM, 28 mM, 26 mM, 24 mM, 22 mM, 20 mM, 15 mM, 10 mM, 5 mM, 4 mM, 3 mM, 2 mM, 1 mM, may be substantially free of fatty acids, or may be free of fatty acids.

The albumin preparation used in the methods described herein, may comprise detergent, such as polysorbate (preferably polysorbate 80) at a concentration less than 200 mg/L, 150 mg/L, 100 mg/L, 90 mg/L, 80 mg/L, 70 mg/L, 60 mg/L, 50 mg/L, 40 mg/L, 30 mg/L, 20 mg/L, 15 mg/L, 10 mg/L, 5 mg/L, 4 mg/L, 3 mg/L, 2 mg/L, 1 mg/L, 0.5 mg/L, 0.1 mg/L, 0.01 mg/L, 0.001 mg/L, may be substantially free of the detergent, or may be free of the detergent.

The albumin preparation used in the methods described herein, may comprise total free amino acid level and/or N-acetyl tryptophan levels less than 35 mM, 32.5 mM, 30 mM, 28 mM, 26 mM, 24 mM, 22 mM, 20 mM, 15 mM, 10 mM, 5 mM, 4 mM, 3 mM, 2 mM, 1 mM, 0.5 mM, 0.1 mM, 0.01 mM, 0.005 mM, 0.001 mM, may be substantially free of free amino acids and/or N-acetyl tryptophan in particular, or may be free of free amino acids and/or of N-acetyl tryptophan in particular.

The albumin preparation used in the methods described herein may be substantially free of, or completely free of, all of octanoate, free amino acids and/or N-acetyl tryptophan in particular, and detergent (such as polysorbate 80).

Typically, the albumin preparation used in the methods described herein, is free of one or more, such as all, components selected from: haem, prekallikrein activator, pyrogens, hepatitis C and/or human viruses. Further, typically, the albumin preparation used in the methods described herein, has an aluminium concentration of less than 200 μg/L, such as less than 180 μg/L, 160 μg/L, 140 μg/L, 120 μg/L, 100 μg/L, 90 μg/L, 80 μg/L, 70 μg/L, 60 μg/L, 50 μg/L, or 40 μg/L, more typically within the range of about 10 μg/L to about μg/L. As used in this context, the term “about”, can include meaning of 10 μg/L, 5 μg/L, 4 μg/L, 3 μg/L, 2 μg/L, 1 μg/L, 0.5 μg/L, 0.1 μg/L or less of the stated value.

Typically, the albumin preparation used in the methods described herein, possesses an intact or substantially intact N-terminal sequence.

Typically, the albumin preparation used in the methods described herein, comprises albumin protein that has a free thiol group content that is greater than about 62%, such as at least about 69%, 70%, 75%, 80%, 85%, 90%, at least about 95%, about 96%, or about 97%. As used in this context, the term “about”, can include meaning of t 50%, 40%, 30%, 20%, 10%, 5%, 4%, 3%, 2%, 1% or less of the stated value; e.g. 80%±10% refers to the range of 72 to 88%.

Typically, the albumin preparation used in the methods described herein, comprises albumin protein that, when tested by size exclusion chromatography (SEC), displays a SEC profile excluding peaks with a peak retention time under 14 minutes and over 19 minutes, and more preferably excludes peaks with a peak retention time under 14 or 15 minutes and over 18 minutes; and/or when tested by reversed phase high performance liquid chromatography (RP-HPLC), displays a single major peak, corresponding to albumin in the native monomeric form; and/or when tested by mass spectrometry, is a product that displays fewer than about 13, about 12, about 11, about 10, about 9, about 8, about 7, about 6, such as about 1 to about 11, 1 to about 8, or 1 to about 5, 1 to about 4, 1 to about 3, 1 to about 2, about 1 or less than 1 hexose modified lysine and/or arginine residues per protein. As used in this context, the term “about”, can include meaning of t 5, 4, 3, 2 or 1 hexose modified lysine and/or arginine residues per protein.

Typically, the albumin preparation used in the methods described herein comprises albumin protein that is not glycated with plant-specific sugars, such as α-1,3-fucose and/or β-1,2-xylose. For the avoidance of doubt, the albumin preparation used in the methods described herein, will be essentially free of, or not contain, plant protein hydrolysate.

Further details on the formulation of preferred recombinant yeast-derived albumin preparations for use in the methods described herein are given in WO 2018/065491, the entire contents of which are incorporated herein by reference.

Preferably, the albumin preparation used in the methods described herein is a recombinant human albumin (rHA) such as Recombumin® Prime (formerly Recombumin®) (sourced from Albumedix Limited) or any preparation that is similar thereto. Preferably the rHA has the following characteristics:
produced by recombinant DNA expression in Saccharomyces cerevisiae;
mass of theoretical mass±20 Da (66418 to 66458) by electrospray mass spectrometry using a standard method known in the art, e.g. United States Pharmacopeia (USP) and National Formulary (NF).
bacterial endotoxin measurement of no greater than 0.5 USP Endotoxin Unit/mL of rHA using a standard method known in the art, e.g. USP <85>;
sterile using a standard method known in the art, e.g. USP <71>;
pH 6.7-7.3 using a standard method known in the art, e.g. USP <791>;
no greater than 1.0% high molecular weight protein impurities using a standard method known in the art, e.g. USP-NF;
no less than 99.0% pure albumin content using a standard method known in the art, e.g. USP-NF (Native PAGE);
total protein of 19.0-21.0% w/v using a standard method known in the art, e.g. USP-NF;
sodium content of 130-160 mM using a standard method known in the art, e.g. USP-NF;
host cell protein impurities no greater than 0.15 μg/g (microgram per gram) using a standard method known in the art, e.g. yeast antigen ELISA;
Concanavalin A binding to rHA no greater than 0.30% (w/w) protein;
nickel content no greater than 0.5 μg/g (microgram per gram) protein;
potassium content no greater than 0.01 mmol/g protein;
octanoate content of 28.8-35.2 mM;
polysorbate content of 10-20 mg/L.

The term ‘aggregation’ as described herein includes the production of multimers (dimers, trimers, tetramers etc.) of albumin. In particular, in a dry state, such as after being spray-dried, proteins such as albumin can begin to cross-link to other molecules using reactive side chains. Such cross-linking can form aggregates, including multimers and polymers.

The term ‘self-aggregation’ as described herein includes aggregation when each of the components undergoing aggregation has the same identity. For example, albumin molecules may aggregate with other albumin molecules (self-aggregate) to form dimers, then trimers, tetramers, higher multimers and polymers.

The term ‘agent that prevents self-aggregation’ as described herein includes any agent that by virtue of its presence reduces, partially or substantially all, of the self-aggregation of albumin compared to the situation when the agent is not present. It will be appreciated that an agent that prevents self-aggregation of albumin (a protein which has evolved to eschew aggregation as a result of being the principal component of blood plasma) will also be likely to prevent non-self aggregation of albumin with other proteins. It will also be appreciated that the presence of an agent that prevents self-aggregation may still permit some of the albumin present to be present in an aggregated form.

The term ‘prevents’ as described herein includes the reduction, partially or substantially all, of a designated phenomenon by a particular product or process compared to the situation where the product was not present or the process was not applied. Albumin has a tendency to self-aggregate under dry conditions and in aqueous solution. Thus, the term ‘prevents self-aggregation of albumin’ as described herein, includes the reduction, partially or substantially all, of the self-aggregation of albumin compared to the background level where a designated or putative preventive agent is not present. It will be appreciated that the self-aggregation of albumin can be measured by any suitable method known in the art, for example gel electrophoresis or chromatography, e.g. high-performance liquid chromatography size exclusion chromatography (HPLC-SEC).

For example, the self-aggregation of albumin may be reduced by at least 10% compared to background level, such as by at least 15%, 20%, 25%, 30%, 35%, 40%, 45%, 50%, 55%, 60%, 65% or 70%, and preferably by at least 75%, 80%, 85%, 90%, 91%, 92%, 93%, 94%, 95%, 96%, 97%, 98%, 99% or 99.5%.

The agent that prevents self-aggregation of albumin may be a sugar, preferably, a non-reducing sugar and/or a sugar with a high glass transition point.

The term ‘sugar’ as described herein includes monosaccharides (one sugar unit), disaccharides (two sugar units), oligosaccharides (up to 10 sugar units) and polysaccharides (>10 sugar units). Monosaccharides may have the general formula CₙH₂ₙOₙ and can be classified by the number of carbon atoms they contain: triose (3), tetrose (4), pentose (5), hexose (6), heptose (7), etc. Generally, they are the fundamental units of carbohydrates and cannot normally be further hydrolyzed to simpler compounds. Monosaccharides include glucose (dextrose), fructose (levulose) and galactose. Disaccharides may consist of two monosaccharides joined by a glycosidic bond. Disaccharides include sucrose (glucose-α-1,2-fructose), lactose (galactose-β-1,4-glucose), maltose (glucose-α-1,4-glucose), trehalose (glucose-α-1,1-glucose), cellobiose (glucose-β-1,4-glucose) and isomaltose (glucose-α-1,6-glucose). Oligosaccharides and polysaccharides, such as cellulose and starch, may also be made up of many monosaccharides. However, it will be appreciated that a sugar molecule for use in the methods and compositions of the present invention must be soluble in aqueous solvents. Both D- and L-isomers of sugars are suitable for use in the methods and compositions of the present invention. Modified sugars and sugar derivatives, e.g. amino sugars (a 2-amino-2-deoxysugar, a sugar molecule in which a hydroxyl group has been replaced with an amine group; such as glucosamine, N-acetylglucosamine, galactosamine, N-acetylgalactosamine), sugar acids (monosaccharides with a carboxyl group; such as gluconic acid, ascorbic acid, glucuronic acid, tartaric acid), sugar alcohols (typically those with the general formula HOCH₂(CHOH)ₙCH₂OH; such as glycerol, xylitol, sorbitol, inositol, maltitol), deoxy sugars (sugars with a hydroxyl group replaced with a hydrogen atom; such as deoxyribose, fucose, fuculose, rhamnose), and sugar phosphate esters (such as glucose-1-phosphate, ribose 5-phosphate), may also be suitable for use in the methods and compositions of the present invention. Further discussion of sugars, modified sugars and sugar derivatives can be found in Chapter 7, pages 203-246 of Garrett & Grisham, Biochemistry, 3^{rd} edition, Thomson Brooks/Cole, 2005, ISBN 0-534-41020-0, the entire contents of which are incorporated herein by reference.

The term ‘reducing sugar’ as described herein includes the meaning of a sugar that is capable of acting as a reducing agent because it has a free aldehyde group or ketone group. The common dietary monosaccharides galactose, glucose and fructose are all reducing sugars, as are some disaccharides, oligosaccharides and polysaccharides. The monosaccharides may be divided into two groups: the aldoses, which have an aldehyde group, and the ketoses, which have a ketone group. Generally, ketoses must first tautomerize to aldoses before they can act as reducing sugars. Reducing sugars may react with amino acids in the Maillard reaction, a series of reactions that occurs while cooking food at high temperatures (the reactions typically proceed rapidly from around 140 to 165° C.) and gives browned food its distinctive flavour. A similar process may also take place when one or more proteins and one or more reducing sugars are present in a spray-dried composition, and therefore it is preferable that the sugar for use in the methods and compositions of the present invention as an agent that prevents self-aggregation of albumin is a non-reducing sugar.

The term ‘non-reducing sugar’ as described herein includes the meaning of a sugar that does not act as a ‘reducing sugar’. Non-reducing disaccharides like sucrose and trehalose have glycosidic bonds between their anomeric carbons and thus cannot normally convert to an open-chain form with an aldehyde group; they are stuck in the cyclic form. Glycogen is a highly branched polymer of glucose that serves as the main form of carbohydrate storage in animals. Although glycogen is a reducing sugar, it has only one reducing end because each branch ends in a non-reducing sugar residue.

As described herein, an amorphous state includes one where a solid has no crystalline structure. It is understood that a solid with an amorphous state may have enhanced solubility and/or dissolution compared to the same substance having a crystalline structure. Some amorphous solids may exhibit a glass transition and so can be called a glass.

The term ‘glass transition’ as described herein includes the meaning of the glass-liquid transition, which is the reversible transition in amorphous materials (or in amorphous regions within semi-crystalline materials) from a hard and relatively brittle ‘glassy’ state into a viscous or rubbery state as the temperature is increased.

The term ‘glass transition point’, also known as the glass transition temperature (T_{g}), as described herein includes the meaning of the mean temperature at which glass transition of an amorphous material (a glass) takes place. It is understood that the T_{g} is always lower than the melting temperature (Tₘ) of the crystalline state of the material, if one exists. Generally, the higher the glass transition point, the more stable the formulation. It is preferable for formulations to be stored 20° C. below their T_{g}.

The term ‘high glass transition point’ as described herein includes temperatures greater than 80° C. An example of a sugar with a high glass transition point is trehalose, as the T_{g} of pure dry trehalose is understood to be 106° C. (Roe & Labuza, Int J Food Prop, 2005, 8(3):559-574). In contrast, the T_{g} of pure dry sucrose is understood to be 60° C. (Roe & Labuza, Int J Food Prop, 2005, 8(3):559-574). Preferably, the glass transition point for an agent that prevents self-aggregation of albumin is greater than 40° C. Most preferably, the agent that prevents self-aggregation of albumin has a high glass transition point, i.e. greater than 80° C. Where an agent that prevents self-aggregation of albumin is used that has a glass transition point that is lower than 40° C., it is preferable to store the solutions and spray-dried powders of the invention at sub-ambient temperatures, e.g. in a refrigerator.

Preferably, the agent that prevents self-aggregation of albumin is selected from one or more of trehalose, sucrose and dextrose, most-preferably trehalose. Trehalose, also known as mycose or tremalose, is a natural alpha-linked disaccharide formed by an α,α-1,1-glucoside bond between two α-glucose units. Because trehalose is formed by the bonding of two reducing aldehyde groups, it normally has no capacity to participate in the Maillard reaction and is therefore a non-reducing sugar. The α,α-1,1-glucoside bond makes trehalose very resistant to acid hydrolysis, and therefore trehalose may be stable in solution at high temperatures, even under acidic conditions. The α,α-1,1-glucoside bond also keeps trehalose in closed-ring form, such that the aldehyde end groups do not bind to the lysine or arginine residues of proteins in the process of glycation. Trehalose is implicated in anhydrobiosis, the ability of plants and animals to withstand prolonged periods of desiccation. Trehalose may be nutritionally equivalent to glucose, because it may be rapidly broken down into glucose by the enzyme trehalase, which may be present in the brush border of the intestinal mucosa of omnivores (including humans) and herbivores. As is evident at least from the examples, the inventors have found trehalose to have particularly useful properties as an agent that prevents self-aggregation of albumin. Trehalose is particularly useful in this regard when the albumin is recombinant human albumin.

Sucrose is a disaccharide formed by a β-1,2-glucoside bond between glucose and fructose, and is a reducing sugar. Sucrose is common table sugar. Sucrose may be stable so that spontaneous hydrolysis happens only very slowly, over several years. Hydrolysis of sucrose to glucose and fructose can be accelerated with acids.

Dextrose, also known as glucose or D-glucose, is a hexose sugar, a monosaccharide, and a reducing sugar. Dextrose can be produced via the enzymatic hydrolysis of starch.

The agent that prevents self-aggregation of albumin may be a polymer, such as a synthetic polymer or a natural polymer. The term ‘polymer’ as described herein includes the meaning of a macromolecule composed of many repeating subunits. It will be appreciated that the term ‘polymer’ also includes branched chain polymers and polymers that have been chemically modified. Possible polymers include sugar polymers and polysaccharides, e.g. starch. Preferably, the polymer for use as an agent that prevents self-aggregation of albumin is suitable for parenteral delivery, e.g. intravenous administration.

The term‘mixing’ as described herein means combining two or more components together, and may additionally comprise stirring, shaking, sonicating and/or whisking. However, when one of the components is albumin, the term ‘mixing’ preferably does not comprise shaking, sonicating and/or whisking.

In the methods described herein, it will be appreciated that there is a wide range of suitable ratios of the Class II or Class IV low solubility molecule to albumin. The ratio of low solubility molecule to albumin may be greater than 1:50, 1:40, 1:30, 1:25, 1:20, 1:15, 1:10, 1:8, 1:6, 1:5, 1:4, 1:3, 1:2.5, 1:2, 1:1.5, 1:1, 1.5:1, 2:1, 2.5:1, 3:1, and 4:1 (w/w). The ratio of low solubility molecule to albumin may be less than 5:1, 4:1, 3:1, 2.5:1, 2:1, 1.5:1, 1:1, 1:1.5, 1:2, 1:2.5, 1:3, 1:4, 1:5, 1:6, 1:8, 1:10, 1:15, 1:20, 1:25, 1:30, and 1:40 (w/w). Preferably, the ratio of low solubility molecule to albumin is greater than approximately 1:50 (w/w), or less than 5:1 (w/w), or between 1:50 and 5:1 (w/w).

In the methods described herein, the solution prior to spray-drying preferably comprises water to maintain the albumin in solution. The mixture of the Class II or Class IV molecule, water, water-miscible solvent (e.g. ethanol), albumin and agent that prevents self-aggregation of albumin may contain at least 40%, 50%, 60%, 65%, 70%, 75%, 80%, 85%, 90% or 95% water prior to spray-drying. In a preferred embodiment, the Class II or Class IV low solubility molecule is dissolved in a mixture of water and a water-miscible solvent (e.g. ethanol) prior to spray-drying. The albumin may be in aqueous solution prior to the methods described herein, i.e. in solution with a solvent comprising water. An albumin solution that exists prior to the methods described herein may comprise water and one or more water-miscible solvents, e.g. ethanol.

It will be appreciated that, in embodiments where the method comprises more than one step prior to spray-drying, there may be a difference in the solvent(s) and/or ratio of solvent(s) between two or more of the steps, and/or between the steps of any such multi-step method and any method with only a single-step prior to spray-drying. For example, in a multi-step method prior to spray-drying, step (a) may comprise dissolving the Class II or Class IV low solubility molecule in a water-miscible solvent(s) to form a solution, step (b) may comprise mixing the solution of the Class II or Class IV low solubility molecule and water-miscible solvent with albumin that is present in an aqueous solution, and step (c) may comprise adding an agent that prevents self-aggregation of albumin to the mixture resulting from step (b); in this example, the solvent or mixture of solvents in each of steps (a), (b) and (c) prior to spray-drying is different, but in another example, the same mixture may be achieved by combining all components together in one step (which will therefore have the same solvent mixture as the solution at the end of step (c) of the multi-step method prior to spray-drying).

In the methods described herein, the solution prior to spray-drying is preferably a single-phase solution of the low solubility molecule, water-miscible solvent(s), albumin and the agent that prevents self-aggregation of albumin, and is most preferably a single-phase solution of the low solubility molecule, water-miscible solvent(s), water, albumin and the agent that prevents self-aggregation of albumin. The term ‘single-phase solution’ as described herein includes the meaning that the solution is homogeneous, for example it has no visible particles and is not a suspension. The term ‘single-phase solution’ as described herein also includes that all of the solvents are miscible together. As such, in a single-phase solution the solvents do not partition (separate), unlike the mixture of some organic solvents (e.g. dichloromethane) with water, where a physical separation takes place after mixing. Among other reasons, having a single-phase solution permits the low solubility molecule to be in close proximity with the albumin on spray drying.

In some embodiments of the methods described herein, the mixture comprising the low solubility molecule, water-miscible solvent, albumin and an agent that prevents self-aggregation of albumin, also comprises a solubility-enhancing agent prior to spray drying. In other embodiments of the methods described herein, the mixture comprising the low solubility molecule, water-miscible solvent, albumin and an agent that prevents self-aggregation of albumin, does not also comprise a solubility-enhancing agent prior to spray-drying.

The term ‘solubility-enhancing agent’ as described herein includes the meaning of an agent other than albumin that, separately or in addition to the albumin, enhances the solubility of the low solubility molecule in aqueous solution (i.e. in a solution that comprises water). It will be appreciated that one or more solubility-enhancing agents may be used in the mixture of the method according to the first aspect of the invention prior to spray drying.

Solubility-enhancing agents as described herein include cyclodextrins, dispersants and surfactants.

The term ‘cyclodextrins’, or cycloamyloses, as described herein may relate to a family of compounds made up of sugar molecules bound together in a ring (i.e. cyclic oligosaccharides). Cyclodextrins may be produced from starch by means of enzymatic conversion. Cyclodextrins may be composed of 5 or more α-D-glucopyranoside units linked by α-1,4-glycosidic bonds, as in amylose (a fragment of starch). The 5-membered macrocycle is understood not to occur naturally. The largest well-characterized cyclodextrin contains 32 1,4-anhydroglucopyranose units, and at least 150-membered cyclic oligosaccharides are also known as part of a poorly characterised mixture. Typical cyclodextrins contain 6-8 glucose monomers in a ring: α (alpha)-cyclodextrin is a 6-membered sugar ring molecule, and is a soluble dietary fibre; β (beta)-cyclodextrin is a 7-membered sugar ring molecule; γ (gamma)-cyclodextrin is a 8-membered sugar ring molecule. α-cyclodextrin and γ-cyclodextrin are currently used in the food industry. Cyclodextrins may form complexes with hydrophobic compounds because cyclodextrins are typically hydrophobic inside and hydrophilic outside. Formation of complexes between cyclodextrins and hydrophobic compounds may enhance the solubility of such hydrophobic compounds (Morrison et al, Mol Pharmaceutics, 2013, 10(2):756-762). Cyclodextrins can also enhance drug permeability through mucosal tissues (Morrison et al, Mol Pharmaceutics, 2013, 10(2):756-762).

The term ‘dispersants’ includes the meaning of either a non-surface active polymer or a surface-active substance added to a suspension, usually a colloid, to improve the separation of particles and to prevent settling or clumping. The term colloid includes a homogeneous non-crystalline substance consisting of large molecules or ultramicroscopic particles of one substance dispersed through a second substance. Colloids include gels, sols, and emulsions; typically the particles do not settle and cannot be separated out by ordinary filtering or centrifuging like those in a suspension. Therefore, a dispersant may aid in making a suspension homogeneous, i.e. amorphous. Dispersants may consist of one or more surfactants.

The term ‘surfactants’ as described herein includes compounds that lower the surface tension (or interfacial tension) between a liquid and a solid. Surfactants may act as detergents, wetting agents, emulsifiers, foaming agents, and dispersants. Surfactants as described herein include polysorbate 80 and polysorbate 20.

In the methods described herein, the solution(s) and/or mixture(s) may be sterilised before or after any step(s) prior to spray drying. For example, where the method comprises multiple steps before spray-drying and one of those steps comprises dissolving the Class II or Class IV low solubility molecule in a water-miscible solvent to form a solution, said solution may be sterilised before the subsequent step(s) at least comprising addition of the albumin and an agent that prevents self-aggregation of albumin. As a further example, where the method comprises a single step before spray-drying (i.e. mixing all of the components together to form the solution or mixture that will be spray-dried), the solution or mixture resulting from that step may be sterilised before spray-drying. The term ‘sterilise’ as described herein includes removing unwanted material, particularly bacteria. One suitable method of sterilisation for use in the methods described herein is sterile filtration, such as using a 0.2 μm (micron) sterile filter. Sterile filtration can be performed on any solution produced prior to spray-drying. It will be appreciated that other sterilisation methods, such as those that are well known in the art, may be suitable for use in the methods described herein.

It will be appreciated that, particularly in embodiments where the method comprises multiple steps before spray drying, it may be advantageous to add additional water-miscible solvent to the solution or mixture that already comprises a water-miscible solvent(s), prior to spray drying; either (1) the same identity of water-miscible solvent as is present in the solution or mixture and/or was used in an earlier step of the method, or (2) a different identity of water-miscible solvent. Preferably, the water-miscible solvent that is additionally added before spray drying is ethanol.

For example, in an embodiment where the method comprises (a) dissolving the Class II or Class IV low solubility molecule in a water-miscible solvent to form a solution, (b) mixing the solution of the Class II or Class IV low solubility molecule and water-miscible solvent with albumin and an agent that prevents self-aggregation of albumin, and (c) spray-drying the mixture; additional water-miscible solvent (either the same identity as the water-miscible solvent in step (a) or a different water-miscible solvent than in step (a)) may be added between steps (b) and (c) of the method.

A step of all of the methods described herein is to spray-dry the mixture (preferably as a solution) of at least the Class II or Class IV low solubility molecule, water-miscible solvent (e.g. ethanol), albumin and an agent that prevents self-aggregation of albumin (which mixture may also include any optional component as described above). The result of the methods described herein is therefore that spray-drying the mixture produces a spray-dried composition. Preferably, this spray-dried composition is amorphous.

The term ‘spray-drying’ as described herein refers to a method of producing a dry powder from a liquid or slurry (hereafter ‘feed solution’) by rapidly drying with a hot gas, and the term ‘spray-dried composition’ as described herein refers to a dry composition produced by spray-drying. The apparatus used for spray-drying is called a ‘spray dryer’.

In summary, a spray dryer may take a liquid stream (feed solution) and separate the solute(s) or suspension(s) as a solid and the solvent(s) into a vapour. The solid is usually collected in a drum or cyclone. The feed solution is typically sprayed through a nozzle into a hot gas stream and vaporised. Typically, solids form as solvent quickly leaves the droplets. A nozzle is usually used to make the droplets as small as possible, maximizing heat transfer and the rate of vaporisation. Droplet sizes can range from 10 to 500 μm (micron) depending on the nozzle. There are at least two main types of nozzles: high pressure single fluid nozzle (50 to 300 bars) and two-fluid nozzles where one nozzle delivers the feed solution and the second nozzle delivers a compressed gas for atomisation of the feed solution. With a two-fluid nozzle, the gas stream impinges on the feed solution stream, thus atomising it. Although the hot gas used in spray-drying is usually air (e.g. at 1 to 7 bars pressure), if the feed solution comprises a flammable solvent such as ethanol or the product is oxygen-sensitive then nitrogen can be used instead.

Spray-drying is the preferred method of drying for many thermally-sensitive materials such as foods and pharmaceuticals because spray-drying may dry a product very quickly compared to other methods of drying, and typically produces a powder from a solution or slurry in a single step. Spray-drying also typically produces a consistent particle size distribution. Its use of evaporative cooling typically keeps the droplet at low ambient temperatures during the drying process

Generally, all spray dryers use some type of atomizer or spray nozzle to disperse the liquid or slurry into a controlled drop size spray. It is understood that the most common ones of these are rotary disk and single-fluid high pressure swirl nozzles. Atomizer wheels may provide broader particle size distribution, but both methods may allow for consistent distribution of particle size. Alternatively, for some applications two-fluid or ultrasonic nozzles can be used. Depending on the process needs, drop sizes from 10 to 500 μm can be achieved with the appropriate choices. The dry powder resulting from spray-drying is often free-flowing. The dry powder resulting from spray-drying with a small-scale spray dryer is typically 1-10 μm (micron) diameter.

Preferred spray dryers are ‘single effect spray dryers’, which may have a single source of drying air at the top of the chamber. In most cases the air is blown in the same direction as the sprayed liquid (co-current). Typically, a fine powder is produced. Other spray dryers include ‘multiple effect spray dryers’ which, instead of drying the liquid in one stage, may perform drying through two steps, typically the first drying step at the top (as per single effect) and the second drying step with an integrated static bed at the bottom of the chamber. However, the humid environment of the bed may cause smaller particles to clump and partially dissolve, and so it will be appreciated that multiple effect spray dryers are not preferred.

The hot drying gas can be passed in as a co-current (same direction as sprayed liquid atomizer) or counter-current (where the hot air flows against the flow from the atomizer). With co-current flow, particles typically spend less time in the system and the particle separator (typically a cyclone device). With counter-current flow, particles typically spend more time in the system, which is usually paired with a fluidized bed system. Co-current flow generally allows the system to operate more efficiently, and so is preferred.

In the methods described herein, spray-drying the mixture can be performed using any spray dryer (spray-drying machine). Examples of suitable spray dryers include those made by Buchi (e.g. Buchi B-290), ProCepT, Niro and Anhydro.

Alternatively, nano spray dryers that produce particles in the range of 300 nm to 5 μm (micron) with a narrow size distribution may be used, which require a minimal sample amount of only 1 mL.

It will be appreciated that different spray-drying parameters may be used, for example according to which low solubility molecule is present in the mixture. Preferably the atomisation pressure is between about 0.5 bar to about 8 bar. Preferably the outlet (drying) temperature is between approximately 40° C. and approximately 120° C. Preferably the outlet (drying) temperature should be kept substantially constant. Depending on the spray dryer used, the inlet temperature used may be dependent on the chosen outlet (drying) temperature. Most preferably the spray-drying is performed using an inlet temperature of approximately 100° C., an outlet temperature of approximately 65° C., a liquid feed rate of about 2 ml/min and an atomisation pressure of about 3 bar.

A second aspect of the invention provides a method of preparing a spray-dried composition comprising (i) a Class II or Class IV low solubility molecule, (ii) albumin, and (iii) an agent that prevents self-aggregation of albumin. The method according to the second aspect of the invention comprises spray-drying a mixture comprising the Class II or Class IV low solubility molecule, a water-miscible solvent, albumin and an agent that prevents self-aggregation of albumin.

It will be appreciated that the order for combining the components of the mixture prior to spray-drying may be varied, as described above according to the first aspect of the invention.

For example, in some embodiments, the method comprises (a) dissolving the Class II or Class IV low solubility molecule in a water-miscible solvent to form a solution, (b) mixing the solution of the Class II or Class IV low solubility molecule and water-miscible solvent with albumin and an agent that prevents self-aggregation of albumin, and (c) spray-drying the mixture.

Preferably the spray-dried compositions described herein are suitable for parenteral delivery, for example subcutaneous delivery, intramuscular delivery, ocular delivery, pulmonary delivery and/or nasal delivery. The term ‘parenteral’ described herein includes a reference to a route of delivery that is not by the enteral route, i.e. that parenteral delivery is not via the gastrointestinal tract. Enteral delivery includes oral administration and administration via gastric tube. The term ‘suitable for parenteral delivery’ as described herein includes having low immunogenicity and low toxicity, so that such a spray-dried composition meets accepted pharmaceutical standards when dissolved in a pharmaceutically acceptable solvent, optionally with any additional pharmaceutically acceptable excipients, diluents or additives. It will be appreciated that such pharmaceutically acceptable excipients, diluents or additives are well known in the art.

The spray-dried compositions described herein may comprise one or more solubility-enhancing agents as described above. Alternatively, the spray-dried compositions described herein do not comprise one or more solubility-enhancing agents as described above.

The low solubility molecule, water-miscible solvent, albumin and agent that prevents self-aggregation of albumin according to the first and second aspects of the invention may be as those described above in relation to the first aspect of the invention.

In the methods according to the first and second aspects of the invention, the ratio of low solubility molecule to albumin may be as described above in relation to the first aspect of the invention.

In the methods according to the first and second aspects of the invention, the solution prior to spray drying is preferably a single-phase solution of the low solubility molecule, water-miscible solvent, albumin and an agent that prevents self-aggregation of albumin, as described above in relation to the first aspect of the invention. In a more preferred embodiment, the solution prior to spray drying is a single-phase solution of the low solubility molecule, water, water-miscible solvent, albumin and an agent that prevents self-aggregation of albumin, as described above in relation to the first aspect of the invention.

In the methods according to the first and second aspects of the invention, the solution(s) and/or mixture(s) may be sterilised before or after any step(s) prior to spray drying, as described above in relation to the first aspect of the invention. For example, where the method comprises multiple steps before spray-drying and one of those steps comprises dissolving the Class II or Class IV low solubility molecule in a water-miscible solvent to form a solution, said solution may be sterilised before the subsequent step(s) at least comprising addition of the albumin and an agent that prevents self-aggregation of albumin, as described above in relation to the first aspect of the invention. As a further example, where the method comprises a single step before spray-drying (i.e. mixing all of the components together to form the solution or mixture that will be spray-dried), the solution or mixture resulting from that step may be sterilised before spray-drying, as described above in relation to the first aspect of the invention.

The methods described herein may further comprise dissolving the spray-dried composition in aqueous solution. This aqueous solution may comprise a surfactant, e.g. polysorbate 80. Preferably, the aqueous solution is one that is suitable for parenteral delivery, for example subcutaneous delivery, intramuscular delivery, ocular delivery, pulmonary delivery and/or nasal delivery. The aqueous solution may also comprise pharmaceutically acceptable excipients, diluents or additives, examples of which are well known in the art.

Particles produced during spray drying according to the methods described herein are usually microparticles (typically, but not limited to, 1-10 μm (micron) diameter) in dry form containing a low solubility molecule, albumin and an agent that prevents self-aggregation of albumin. When the dry microparticles dissolve in water, typically the agent that prevents self-aggregation of albumin will dissolve and the low solubility molecule and albumin will form nanoparticles dispersed in the water. The term ‘nanoparticles’ as described herein includes all particles of less than 1 μm (micron) diameter. Preferably, in the methods described herein, the mixture prior to spray drying does not comprise nanoparticles, but is a solution.

Preferably, the albumin within the spray-dried compositions described herein is not substantially denatured or crosslinked. As such, the albumin will have reduced immunogenicity compared to denatured, crosslinked, aggregated or hydrolysed albumin. It will be appreciated that spray-dried compositions described herein wherein the albumin is not denatured or crosslinked may be suitable for parenteral delivery. The extent of the denaturation and/or crosslinking of albumin can be determined by size exclusion chromatography. Additionally, for mixtures comprising at least a low solubility molecule and albumin, the production of a milky suspension may be indicative of the presence of denatured albumin. It will be appreciated that there is a degree of tolerance regarding denaturation and/or crosslinking, so that a small (ideally a negligible) amount of the albumin may be denatured and/or crosslinked without substantially affecting the spray-dried composition or its suitability to be used for parenteral delivery when dissolved in a pharmaceutically acceptable solvent.

A third aspect of the invention provides a spray-dried composition comprising (i) a Class II or Class IV low solubility molecule, (ii) albumin, and (iii) an agent that prevents self-aggregation of albumin. The spray-dried compositions according to the third aspect of the invention may be produced by any of the methods according to the first and/or second aspects of the invention, but it will be appreciated that the spray-dried compositions according to the third aspect of the invention are not limited to the spray-dried compositions produced by such methods.

Preferences for (i) the Class II or Class IV low solubility molecule, (ii) albumin, and (iii) the agent that prevents self-aggregation of albumin include those described above in relation to the first and second aspects of the invention.

Preferably, the spray-dried compositions according to the third aspect of the invention are suitable for parenteral delivery, for example subcutaneous delivery, intramuscular delivery, ocular delivery, pulmonary delivery and/or nasal delivery. It will be appreciated that, like the spray-dried compositions prepared according to the first and second aspects of the invention, the spray-dried compositions according to the third aspect of the invention may also be dissolved in aqueous solution, which may further comprise pharmaceutically acceptable excipients, diluents or additives, examples of which are well known in the art.

A fourth aspect of the invention provides the use of albumin in a spray drying method to enhance the solubility and/or the rate of dissolution of a Class II or Class IV low solubility molecule. Preferably, the use of albumin is in combination with an agent that prevents self-aggregation of albumin (e.g. trehalose). It is preferred that the use of albumin according to the fourth aspect of the invention is achieved by carrying out a method according to the first or second aspects of the invention. Hence, preferably, the fourth aspect of the invention is carried out by a method that includes spray-drying a mixture comprising the albumin, the Class II or Class IV low solubility molecule, a water-miscible solvent and an agent that prevents self-aggregation of albumin.

Preferences for the Class II or Class IV low solubility molecule, albumin and the agent that prevents self-aggregation of albumin include those described above in relation to the first and second aspects of the invention.

A fifth aspect of the invention provides a spray-dried composition according to the third aspect of the invention for use in medicine, in particular, for use as a medicament.

A sixth aspect of the invention provides a spray-dried composition according to the third aspect of the invention for use in treating or preventing a disease or condition in an individual in need thereof. For example, for a given API that is known to treat or prevent a given disease or condition, it may be desirable to enhance the solubility and/or the rate of dissolution of the API, and so it will be appreciated that the present invention includes a spray dried composition of such an API for use in treating or preventing the corresponding disease or condition that the API is known to treat or prevent.

For the avoidance of doubt, the term ‘described herein’ relates to the specification as a whole, and therefore the direction given by this term is not limited to any single portion of the specification or any single aspect of the invention.

The listing or discussion of an apparently prior-published document in this specification should not necessarily be taken as an acknowledgement that the document is part of the state of the art or is common general knowledge.

The invention will now be described in more detail by reference to the following Examples and Figures.

### BRIEF DESCRIPTION OF THE FIGURES

FIG. 1—Schematic flow chart of feed solution preparation used in the Examples (but without limiting the scope of the invention accordingly).

FIG. 2—Images of dissolved formulations 004B, C and D in saline. 004 A and B formed inhomogeneous, flaky suspensions, whereas 004D formed a more homogeneous solution. The hazy appearance is indicative of the presence of nanoparticles.

FIG. 3—Images of dissolved formulations of 004D in deionised (DI) water at AP1903 concentrations of 0.06, 0.15 and 0.30 mg/mL.

FIG. 4—Zetasizer® traces of 004D solution prepared in water, together with the rHA and AP1903 alone.

FIG. 5—A600 traces over 24 hours for formulations 008, 009A and 009B in DI water. Turbidity is generally related to AP1903 concentration. Image of 009B formulation dissolved in DI water at an AP1903 concentration of 0.47 mg/mL.

FIG. 6—A600 traces over 24 hours for formulations 012A, B and C in DI water. A600 values measured correlated with the AP1903 concentration, and no effect of trehalose was observed.

FIG. 7—Images of 012A formulation dissolved in DI water at a range of AP1903 concentrations (upper), and at an AP1903 concentration 0.3 mg/mL over 24 hours (lower). Note concentrations shown refer to total mass of formulation, and correspond to 0.14, 0.34, 0.68 and 1.35 mg/mL AP1903, respectively (upper).

FIG. 8—A600 traces over 24 hours for formulations 013A and B in DI water. No effect of chilling the feed solution on measured A600 values was observed.

FIG. 9—A600 traces over 24 hours for formulations 015A, B and C in DI water. A600 values decrease with AP1903:rHA ratio, but also correlate with the AP1903 concentration used in testing. Taking concentrations into account, overall no significant difference is observed.

FIG. 10—A600 traces over 24 hours for formulations 017A and B in DI water. A600 values for formulation 017B produced at a higher feed solution concentration were lower and more stable overtime.

FIG. 11—Plot of feed solution concentration vs. A600 measurement at 60 min for all samples prepared during the project.

FIG. 12—A600 traces over 24 hours for formulations 018A and B in DI water.

FIG. 13—Plots of A600 vs AP1903 concentration overtime for all formulations. For most formulations A600 correlated with concentration in the same way. The exception to this is 017B (separate trendline indicated on the figure), which deviates away from the group, and has lower A600 values for a given concentration and these are more stable overtime.

FIG. 14—Comparison of the dissolution performance for batches prepared with two different volumes of ethanol at the end of the feed solution preparation. A600 measurements over 24 hours for two formulations prepared with the addition of 1.5 mL (left) and 3.0 mL (right) of ethanol to obtain a visually clear feed solution. Higher volumes of ethanol lead to higher and less stable A600 values. Individual lines represent different concentrations of AP1903 in solution.

FIG. 15—Plot of AP1903 recovery vs concentration of AP1903 in ethanol in the feed solution. A threshold value around 3 mg/mL is found, above which the recovery of AP1903 is reduced as it precipitates out of the feed solution before spray drying.

FIG. 16—A600 plots for batches made with the addition of 0, 1 and 2 mL of ethanol added to the feed solution. Higher added volumes lead to worse A600 performance. Individual lines represent different concentrations of AP1903 in solution.

FIG. 17—A600 plot of 023 over 24 hours in DI water showing low values and good stability. Dissolution performance of optimised formulation confirms the 3:1 molar ratio of rimiducid:rHA where no further ethanol was added to the feed solution. Individual lines represent different concentrations of AP1903 in solution.

FIG. 18—A600 plot of scaled up batches 026A and B over 24 hours in 5% (w/v) dextrose solution (D5W) showing low values and good stability. The results for 026B can be compared to the grey trace shown for batch 023 prepared on the smaller scale in FIG. 17.

FIG. 19—Images of the dissolution of formulation 026B in D5W over 5 hours.

FIG. 20—A600 trace for formulation 026B at an AP1903 concentration of 0.5 mg/ml in D5W over 5 hours. The formulation was initially reconstituted in water prior to being let down into D5W, resulting in some dilution of the D5W. Hence, the final calculated concentration of dextrose shown on the Figure is 4.59% rather than 5%.

FIG. 21—Plots of A600 data averaged over the three repeat batches (036A, B and C) for 24 hours. An optical image of the solution is included for reference.

FIG. 22—A600 traces for formulation 040A and B, and Placebo (041) over 24 hours.

FIG. 23—Zetasizer® traces for formulation 036A pre and post filtration through a 0.45 μm filter. Post filtration peaks were still observed for particles larger than the filter pore size. This shows the transient nature of the structures as they reform post-filtration, showing the presence of particles with size greater than 0.45 μm (micron). Individual lines represent n=3 repeat measurements.

FIG. 24—Zetasizer® traces for formulation 040A and B at a 1:50 dilution of the A600 measurement solution. Larger peaks have disappeared suggesting these were artefacts of the concentrations previously used.

FIG. 25—Zetasizer® traces for formulation 040A over 24 hours.

FIG. 26—Nanosight trace for sample 036B showing multimodal distribution of sizes of nanoparticle present.

FIG. 27—Zetapotential measurements on sample 036B giving a value of −28.1±4.3 mV at pH7. The zeta potential was measured to obtain an idea of the stability of the nanoparticles. The average value obtained over n=3 analyses was −28.1±4.3 mV at pH7, indicating the nanoparticles have moderate stability against flocculation or aggregation. Individual lines represent n=3 repeat measurements.

FIG. 28—Size exclusion chromatography (SEC) traces for stock rHA solution, and of rHA in the formulation pre and post filtration through a 1.2 μm filter.

FIG. 29—A600 traces for formulations 040A (t=0) and 045 (t=7 days at 37° C.). No change is observed after storage.

FIG. 30—DSC traces for formulations 040A (t=0) and 045 (t=7 days at 37° C.).

FIG. 31—Residual moisture by loss on drying before (left) and after (right) storage. No significant difference in residual moisture.

FIG. 32—Photographs of the reconstituted spray-dried (SD) formulations in D5W.

FIG. 33—UV/Vis analysis at 600 nm of the reconstituted formulations over 24 hours. The absorbance scale for the Lopinavir and Phenytoin was set to the lower value of 0.120 as the default 0.700 failed to show the shape of the traces.

FIG. 34—Zetasizer® traces for the reconstituted spray dried powders at approximately 0.3 mg/mL API.

FIG. 35—SEC-HPLC chromatograms of the reconstituted formulations in D5W.

FIG. 36—UV-Vis analysis at 600 nm of the reconstituted formulation over 24 hours.

FIG. 37—Zetasizer® analysis of reconstituted Ezetimibe formulation in D5W solution.

FIG. 38—Size exclusion chromatogram of reconstituted Ezetimibe formulation produced using DMSO.

FIG. 39—Overlay of HPLC-SEC peaks from stability study (t=0, 7, 14, 21, and 28 days).

FIG. 40—Dissolution of curcumin in water (left) and after spray drying with rHA (right).

### EXAMPLES 1-6

Summary of Examples 1-6

A series of experimental studies were undertaken to develop a spray dried formulation containing rimiducid (AP1903), recombinant human albumin (rHA) and trehalose. The dry powder formulation was produced by spray drying all of the ingredients from a single water/ethanol solution, to produce a stable spray dried dispersion (SDD).

The rationale behind this study was to investigate whether the SDD produced would have improved aqueous dispersion/solubility properties compared to the unformulated rimiducid, which is a BCS class IV compound exhibiting poor solubility/bioavailability.

Initial investigations focused on developing a spray drying feed solution in which both the drug and rHA remained in soluble form for long enough to be spray dried without loss of drug/rHA and without damaging either molecule.

Once this was achieved, trehalose was added to the formulation, in order to stabilise the rHA, and a spray drying method was developed to produce the rimiducid:rHA SDD. The eventual aim of these studies was to create a SDD that could be re-dispersed in an aqueous infusion buffer at a rimiducid concentration of at least 0.3 mg/ml.

In addition to these development studies it was also necessary to develop suitable analytical techniques that could determine whether the SDD batches produced exhibited improved solubility properties. Analytical methods were developed to measure the AP1903 loading in the formulation and to quantify the stability and degree of the drug solubilisation over time.

The preparation of the feed solution was found to be key to product performance. Balancing of the solubility of the different components was essential for successful complex formation and creation of a product which dissolved well and was stable over time.

Closer examination of the re-dispersed SDD revealed that rimiducid and rHA formed both soluble complexes and nanoparticles when the spray dried powder went back into aqueous solution.

Results also suggest that the rimiducid:rHA complex was not tightly bound, and that the nanoparticles formed could be transient in nature, such that rimiducid and rHA are constantly fluxing in and out of the larger nanoparticulate complex. However, over a 24 hour period the nanoparticulate suspension did not change in overall size, showing that it was suitable for intravenous administration.

A stability study was undertaken that showed that the spray dried rimiducid:rHA:trehalose SDD was stable over 7 days, when stored at 37° C./ambient RH.

Materials for Examples 1-6

TABLE 1
Materials used for batch production and analysis

| Item | Supplier | Lot/Batch No. |
|---|---|---|
| Recombinant Albumin (rHA) | Albumedix | 1907 |
| AP1903 | Bellicum | RC012090 |
| Trehalose | Hyashibara | 5F261 |
| Ethanol | Fisher Scientific | 1722802 |
| Water | Fisher Scientific | 1724937, 1726806, |
| | | 1731809 |
| Sodium Chloride | Sigma Aldrich | SLBS5149 |
| Dextrose | Sigma Aldrich | SLBQ9668V |
| TFA | Fisher | 1719859 |
| Acetonitrile | Fisher | 1721955 |
| Methanol | Fisher | 1724488 |
| Sodium phosphate mono | Sigma Aldrich | BCBT4829 |
| basic di hydrate | | |
| Sodium phosphate di basic | Sigma Aldrich | BCBT7935 |
| di hydrate | | |
| Sodium sulfate | Sigma Aldrich | SLBR3463V |

### Example 1—Preliminary Investigations

Initial formulation testing was undertaken to set up a method for the preparation of feed solutions, to test the spray drying parameters, to set up the required analytical methods for loading and dissolution and to identify a suitable starting formulation for further development.

Methods

Feed Solution Preparation

AP1903 was dissolved in ethanol at a concentration of 40 mg/mL. The required volume of this solution to give the desired mass of AP1903 was added to a 50:50 solution of ethanol: HPLC grade water with stirring. A dilute solution of rHA was prepared and the AP1903 solution was added dropwise with stirring. During this addition, the solution went from clear to opaque due to precipitation of the AP1903. Further ethanol was titrated back into the solution in 1 mL aliquots until a visually clear solution was attained.

Spray Drying

All batches were spray dried using a Buchi B-290 spray dryer fitted with a Buchi two-fluid spray nozzle. The spray dryer was fitted with a high-performance cyclone. Solutions were pumped into the spray dryer using a Masterflex peristaltic pump. The spray drying conditions used are listed in Table 2.

Loading Measurements

Loading measurements were undertaken based on a bioanalysis method for AP1903 supplied by Southern Research. Samples were acidified by the addition of trifluoroacetic acid (TFA), which denatured the rHA. The rHA and AP1903 were then separated using solid phase extraction. In a typical extraction 10-20 mg of formulation was added to 1 mL of HPLC grade water and mixed to form a solution. 4 mL of 0.1% TFA in acetonitrile was then added to this solution and mixed. Once the solution had gone clear, 1 mL was taken and passed through an Agilent Captiva ND cartridge. The filtrate was then analysed by HPLC using an Agilent 1100 employing an ACE 5 C18-PFP (50×2.1 mm i.d., 5 μm) column, a gradient of 0.005% TFA in H₂O and methanol with a flow rate of 1 mL/min with UV detection at 280 nm (0 mins—30:70, 2.5 min—0:100, 2.6 min—30:70, respectively).

A600 Measurements

The absorption of the samples at 600 nm was measured using a Shimadzu uv-vis spectrophotometer. Samples were prepared by dissolving the spray dried powder in DI water at the concentrations described. Samples were made up in 2 ml polystyrene cuvettes, and the absorbance at 600 nm was recorded at time intervals, up to 24 hours post dissolution. Optical images of the cuvettes were also taken as a record of the visual appearance of the solutions.

Zetasizer® analysis

For nanoparticle size analysis samples were analysed in DI water, at the concentration used in the A600 measurements, using a Malvern Instruments Zetasizer® Nano S. Samples were contained in a 2 mL quartz glass cuvette. The Z-average size, and PDI were measured.

Results

Drug solubility testing was undertaken to ascertain the highest concentration of drug solution that could be used for the preparation of the feed solution. Considering available data on solubility of AP1903, ethanol and methanol were selected for testing as they were compatible with rHA and can be spray dried easily. Both were tested and found to perform similarly, with ˜40 mg of AP1903 dissolving into 1 mLl of solvent. Based on these results, ethanol was selected for further testing as any consequences of residual solvents in the products are lower.

In order to prepare a homogeneous feed solution for spray drying, the AP1903 and rHA needed to be combined together into a single-phase solution. The composition of solvents in this feed solution needed to be chosen so both the AP1903 and rHA were soluble; therefore, sufficient ethanol was required to dissolve the AP1903 but not so much that the rHA was denatured. It was also noted that when adding ethanol to aqueous solutions, the possibility of locally high concentrations of ethanol could occur. Care was taken to avoid this, with constant effective mixing during addition of ethanol to rHA solutions.

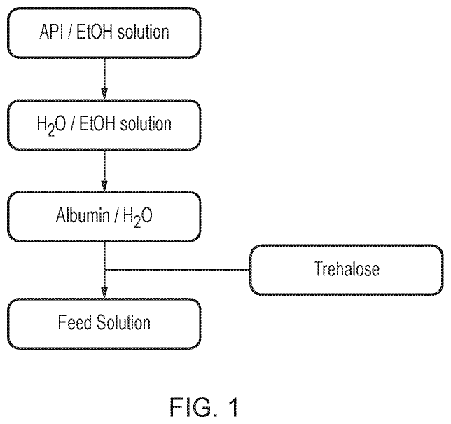

For the initial testing a solution preparation method was found which ensured that the rHA was never exposed to an ethanol concentration greater than 40 v/v %. This limit was chosen to protect the rHA from denaturation. To achieve this, and minimise shocking the rHA with the addition of ethanol, AP1903 was initially dissolved in ethanol at a concentration of 40 mg/ml, a volume of this solution containing the desired mass of AP1903 was then added to a 1:1 v/v water/ethanol mixture. This solution was then added to the dilute rHA solution. In most cases the AP1903 precipitated out during the preparation, and further ethanol was titrated back in until a visually clear solution was formed. Trehalose was then added to the solution to stabilise and protect the rHA (FIG. 1).

Batches were produced using this method covering a range of API loadings from 5.9-20.0 wt %, corresponding to AP1903: rHA molar ratios of 5:1-19:1. Volumes used during the solution preparation were adjusted to ensure the final ethanol content remained below 40 v/v % (Table 2).

Batches were spray dried according to the parameters set out in Table 2. For all batches, the feed solutions were prepared successfully as clear solutions. It was found that additional ethanol was required in some cases, with the addition volume ranging from 3-19 mL as shown in Table 2. This did not appear to correlate to any formulation or preparation characteristics. All batches were found to spray dry successfully, producing free flowing white powders in acceptable yields at this scale, ranging from 27 to 46%.

TABLE 2
Preliminary batches prepared investigation the feed solution preparation and
AP1903 loadings

| | | | | Extra | | | | | |
|---|---|---|---|---|---|---|---|---|---|
| | | | | EtOH | | Liquid | | | |
| | | Batch | API 903 | required | | feed | Inlet | Outlet | Atomisation |
| Experimental | | size | Loading | in feed | AP1903:rHA | rate | temp | temp | Pressure |
| Rationale | Batch | (g) | wt % | (ml) | Molar Ratio | (g/min) | (° C.) | (° C.) | (bar) |
| Testing | 004A | 1600 | 9.1 | 10.0 | 5:1 | 2.0 | 91 | 65 | 3.0 |
| loadings and | 004B | 1100 | 16.7 | 10.0 | 9:1 | 2.0 | 85 | 65 | 3.0 |
| feed solution | 004C | 1140 | 13.2 | 19.0 | 14:1 | 2.0 | 92 | 65 | 3.0 |
| methods | | | | | | | | | |
| preparation | 004D | 850 | 5.9 | 0.0 | 8:1 | 2.0 | 92 | 65 | 3.0 |
| Repeat batch | 008 | 850 | 59 | 0.0 | 8:1 | 2.0 | 111 | 65 | 3.0 |
| 004D | | | | | | | | | |
| Reducing | 009A | 630 | 4.7 | 3.0 | 5:1 | 2.0 | 105 | 65 | 3.0 |
| Loading | | | | | | | | | |
| increasing | 0098 | 1000 | 20.0 | 0.0 | 19:1 | 2.0 | 105 | 65 | 3.0 |
| Loading | | | | | | | | | |

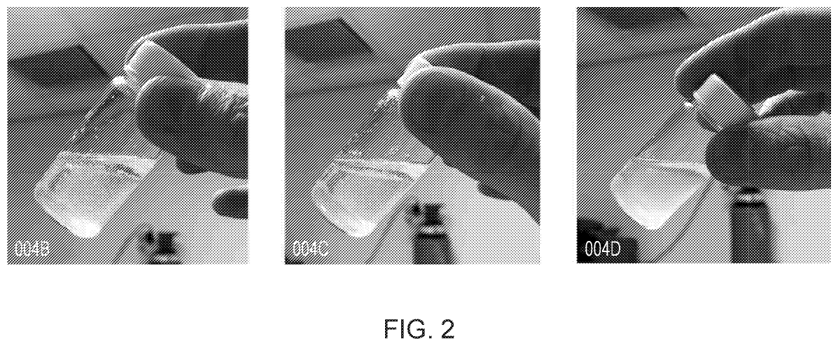

The dissolution of the initial series of batches (004A-D) was tested firstly in saline, as the product would need to be dissolved in an isotonic solution for administration. Sufficient spray dried material was weighed out to give a 0.24 mg/mL AP1903 concentration following dissolution, and the required volume of saline was added to the powder. Visual assessment of the solutions showed that the higher loaded batches did not dissolve well, and formed flaky, inhomogeneous suspensions. In contrast, the lowest loaded batch 004D produced a more homogeneous solution, which had a hazy appearance indicating the likely presence of nanoparticles (FIG. 2).

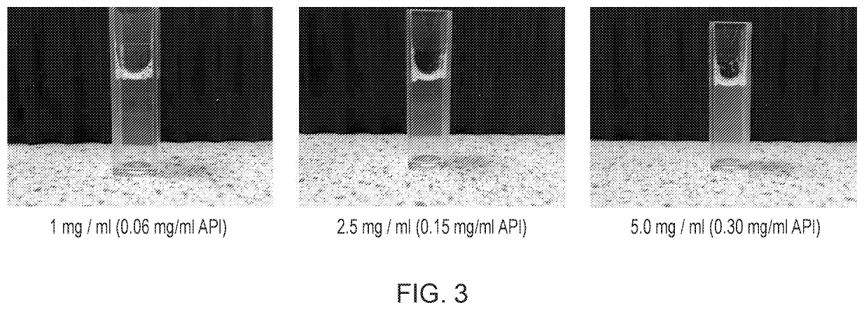

It was noted that use of saline might not be optimum; therefore, 004D was tested further in DI water at a range of concentrations, giving 0.06, 0.15 and 0.30 mg/mL AP1903. It was found that the formulation dissolved more readily into DI water than saline, and that at all concentrations a clear solution was formed. The haziness noted previously was present, and was concentration dependent, being barely visible at 0.06 mg/mL and clearly apparent at 0.30 mg/mL (FIG. 3).

The loading of AP1903 was measured in the spray dried powder using a solid phase extraction method, following denaturation of the rHA under acidic conditions. A loading of 93.6±1.4% of the expected nominal loading was found, indicating that the formulation contained very close to the expected amount of AP1903.

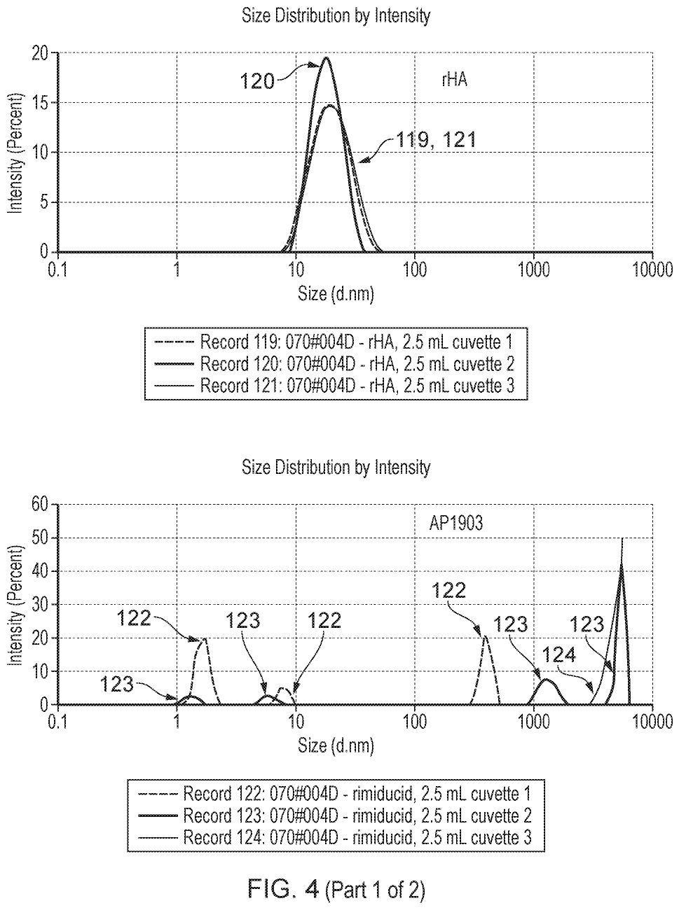

In order to investigate the presence of nanoparticles in the solutions further, initial Zetasizer® measurements were taken on the solutions prepared in water, together with the rHA and AP1903 alone. These indicated that the combination of the AP1903 and rHA was indeed forming nanoparticles, and these were different from those found in either of the starting materials (FIG. 4).

Following these results, the formulation 004D was tested further, and additional material was prepared, as formulation 008. Alongside this, the effects of AP1903 loading was investigated, with lower and higher loaded batches, 009A and B respectively. All of these batches spray dried well, and it was noted that in the case of 008, no further addition of ethanol was required to achieve a clear feed solution during preparation, as the AP1903 remained in solution throughout the addition to aqueous rHA solution.

Following spray drying the loading of AP1903 was again measured in the spray dried powders from batches 009A and B using the same method. Loadings of 99 and 95% vs the expected nominal loadings were obtained respectively.

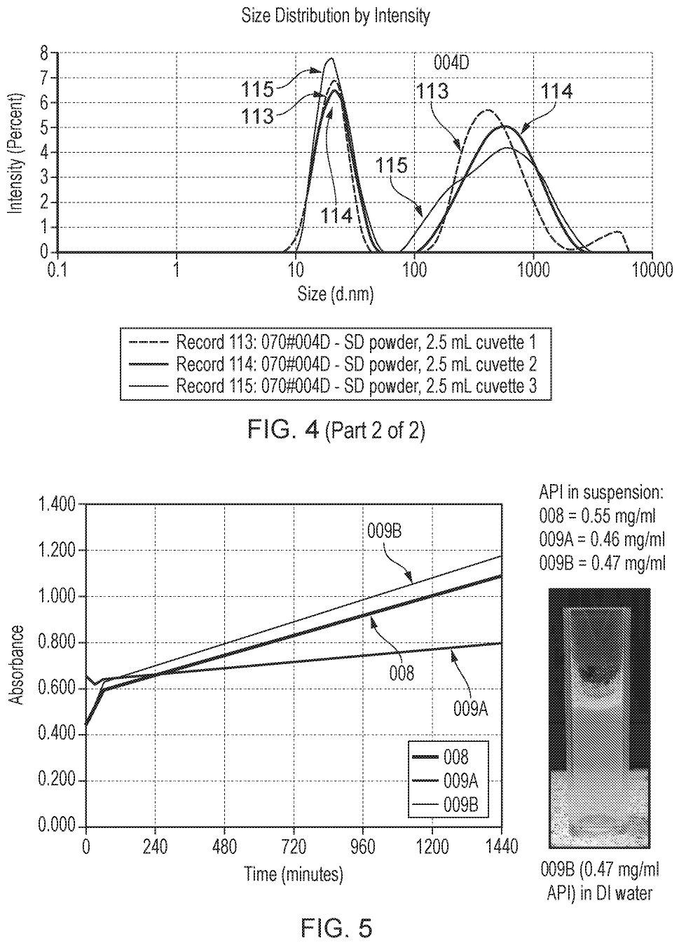

Dissolution of these formulations was assessed in DI water, and the turbidity of the resulting solutions quantified by the absorption of visible light at 600 nm. A more opaque solution was taken as an indicator of poorer dissolution, as this would appear as more turbid, with a higher A600 value. The solutions were dissolved at a concentration of 10 mg/mL formulation, which corresponded to 0.18, 0.46 and 1.90 mg/mL AP1903 for formulations 008, 009A and 009B respectively. It was found from plotting A600 vs time over 24 hours that higher concentration of AP1903 (009B) gave a high A600 value, which was corroborated as being highly opaque by visual assessment (FIG. 5). The A600 results from 008 and 009A were comparable, with the 009A formulation giving a solution which appeared more stable over time (FIG. 5).

Conclusions

Based upon the data above, the formulation prepared as batches 004D/008 was identified as the best performing formulation, based on its dissolution properties and achievable AP1903 loading. This formulation was therefore chosen as the basis for further, more in-depth investigation.

### Example 2—Process and Formulation Optimisation

From the preliminary investigation, the formulation prepared as batch 004D, and repeated as 008, was chosen to take forward. A range of process and formulation parameters were identified for testing, and taking the 004D/008 formulation as a basis, these were systematically tested for influence on the dissolution performance of the formulation.

Methods

Feed Solution Preparation

AP1903 was dissolved in ethanol at a concentration of 40 mg/mL. The required volume of this solution to give the desired mass of AP1903 was added to a 50:50 solution of ethanol: HPLC grade water with stirring. A dilute solution of rHA was prepared and the AP1903 solution was added dropwise with stirring. During this addition, the solution went from clear to opaque due to precipitation of the AP1903. Further ethanol was titrated back into the solution in 1 mL aliquots until a visually clear solution was attained.

Spray Drying

All batches were spray dried using a Buchi B-290 as described in the corresponding section of Example 1 Methods above. The spray drying conditions used in the testing of each parameter are listed in the relevant batch table.

Loading Measurements

Loading measurements were undertaken as described in the corresponding section of Example 1 Methods.

A comparison between the Agilent Captiva ND cartridge and a Phree Filtration tube was made during these experiments, and the Phree filtration tube was found to be less efficient at separating the AP1903 from the rHA, so was not used for further experiments.

A600 Measurements

The absorption of the samples at 600 nm was measured as described in the corresponding section of Example 1 Methods.

Effect of Trehalose

Trehalose was added to the formulation to stabilise and protect the rHA component. The amount of trehalose was initially chosen to match w/w the mass of rHA present. This was varied to test any effect the trehalose may be having on the wider formulation. During the preparation of these batches, the other parameters such as AP1903:rHA molar ratio were kept constant, so the AP1903 loading, and the mass of material processed necessarily changed to accommodate the differing amounts of trehalose (Table 3).

TABLE 3
Batches prepared with a range of trehalose loadings to test the effects of
trehalose on product performance

| | | | | Extra | | | | | |
|---|---|---|---|---|---|---|---|---|---|
| | | | | EtOH | | Liquid | | | |
| | | Batch | AP1903 | required | | feed | inlet | Outlet | Atomisation |
| Experimental | | size | Loading | in feed | AP1903:rHA | rate | temp | temp | Pressure |
| Rationale | Batch | (mg) | wt % | (ml) | Molar Ratio | (g/min) | (° C.) | (° C.) | (bar) |
| Adding 20 mg | 012A | 400 | 13.5 | 0.0 | 8:1 | 2.0 | 102 | 65 | 3.0 |
| Trehalose | | | | | | | | | |
| Adding 200 | 012B | 600 | 9.1 | 0.0 | 8:1 | 2.0 | 101 | 65 | 3.0 |
| mg Trehalose | | | | | | | | | |
| Adding 2000 | 012C | 2400 | 2.1 | 0.0 | 8:1 | 2.0 | 101 | 65 | 3.0 |
| mg Trehalose | | | | | | | | | |

All batches were found to process well, with yield obtained ranging from 38 to 71%. The yield was found to correlate with batch size, indicating that losses are primarily fixed, and reduced with scale.

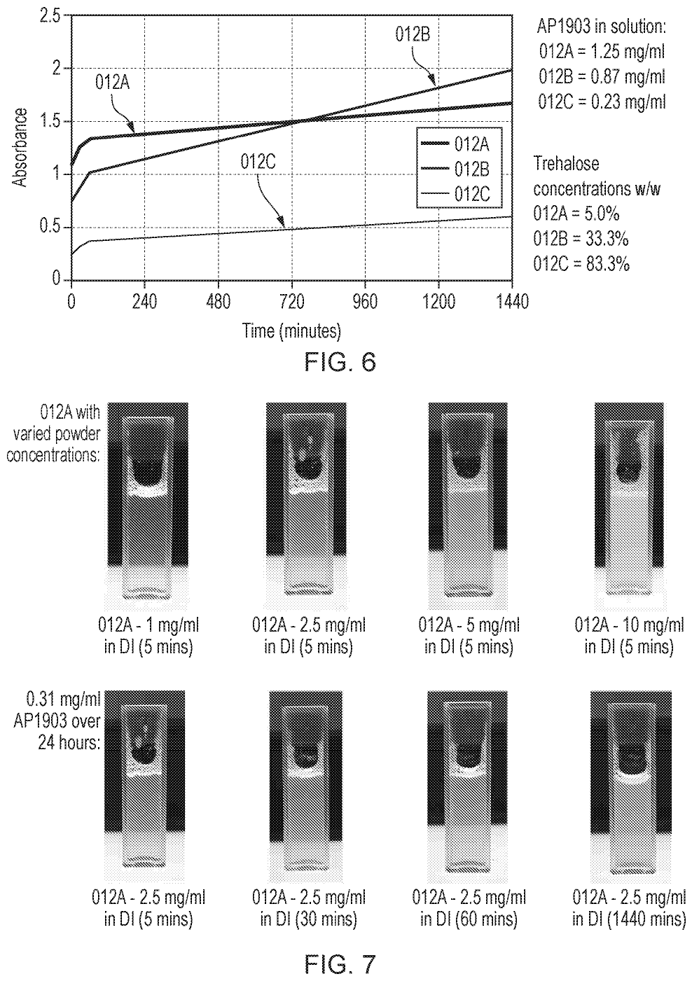

The dissolution of the formulations in DI water was tested at AP1903 concentrations corresponding to 1.25, 0.87 and 0.23 mg/mL for 012A, B and C respectively. Differences in the A600 values over time were observed, but these appeared to correlate to the AP1903 loadings tested (FIG. 6), and overall no effect of trehalose was observed.

The loading of AP1903 was measured in the spray dried powders from batches 012A, B and C using the solid phase extraction method. Loadings of 93, 95 and 109% vs the expected nominal loadings were obtained respectively.

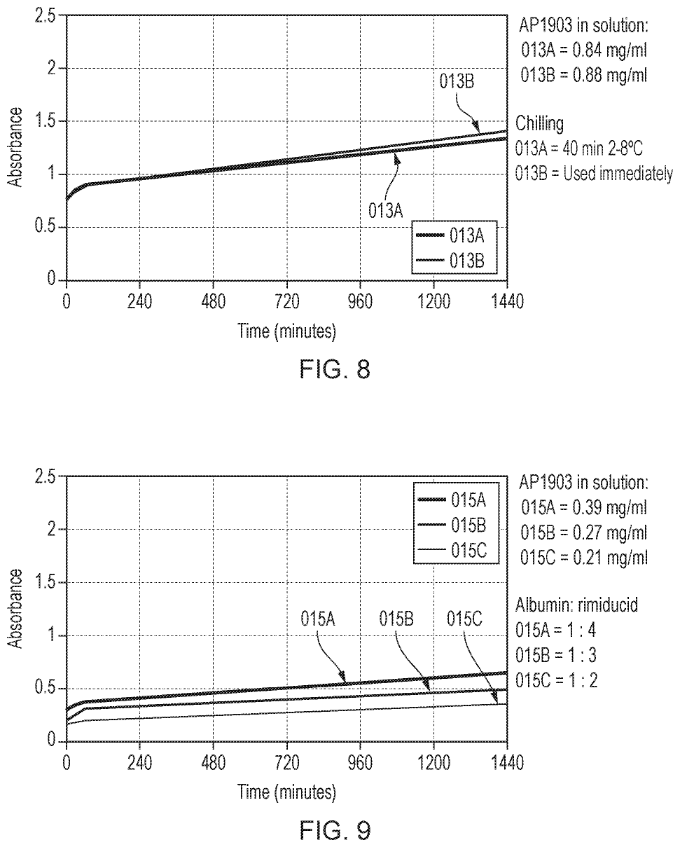

An interesting comparison was made using visual observation of the 012A batch at both a range of concentrations, and over time at a fixed concentration (FIG. 7). It can be observed that the opacity of the solution increases with concentration as may be expected, but over 24 hours no great change was observed. This observation backs up the A600 measurements.

Chilling Feed Solution

As the process of forming the AP1903/rHA complex involves the binding of components present in a co-solvent solution, the key to driving the complex formation is whether the bound components are more thermodynamically favourable than the unbound components. All prior solutions were chilled at 2-8° C. for 40 minutes with the aim of reducing the solubility of the AP1903, and forcing complex formation.

To test if this step was necessary, two batches were prepared: the feed solution was left at room temperature for 40 minutes (013A) and the feed solution was spray dried immediately after preparation (013B) (Table 4). Both batches processed well, with free-flowing powders being recovered in yields of 40 and 46% for batches 013A and 013B respectively. Comparing the yields to those obtained previously, it did not appear that chilling of the solution had any effect on the processing.

The loading of AP1903 was measured in the spray dried powders from batches 013A and B using the solid phase extraction method. Loadings of 93 and 97% vs the expected nominal loadings were obtained, indicating the formulations contained the desired loadings of AP1903.

TABLE 4
Batches prepared with and without chilling to test the effects of chilling the feed
solution on product performance

| | | | | Extra | | | | | |
|---|---|---|---|---|---|---|---|---|---|
| | | | | EtOH | | Liquid | | | |
| | | Batch | AP1903 | required | | feed | inlet | Outlet | Atomisation |
| Experimental | | size | Loading | in feed | AP1903:rHA | rate | temp | temp | Pressure |
| Rationale | Batch | (mg) | wt % | (ml) | Molar Ratio | (g/min) | (° C.) | (° C.) | (bar) |
| Feed solution | 013A | 600 | 14 3 | 0 0 | 8:1 | 2.0 | 105 | 65 | 3.0 |
| Chilled | | | | | | | | | |
| Feed solution | 0138 | 600 | 14.3 | 0.0 | 8:1 | 2.0 | 110 | 65 | 3.0 |
| used | | | | | | | | | |
| immediately | | | | | | | | | |

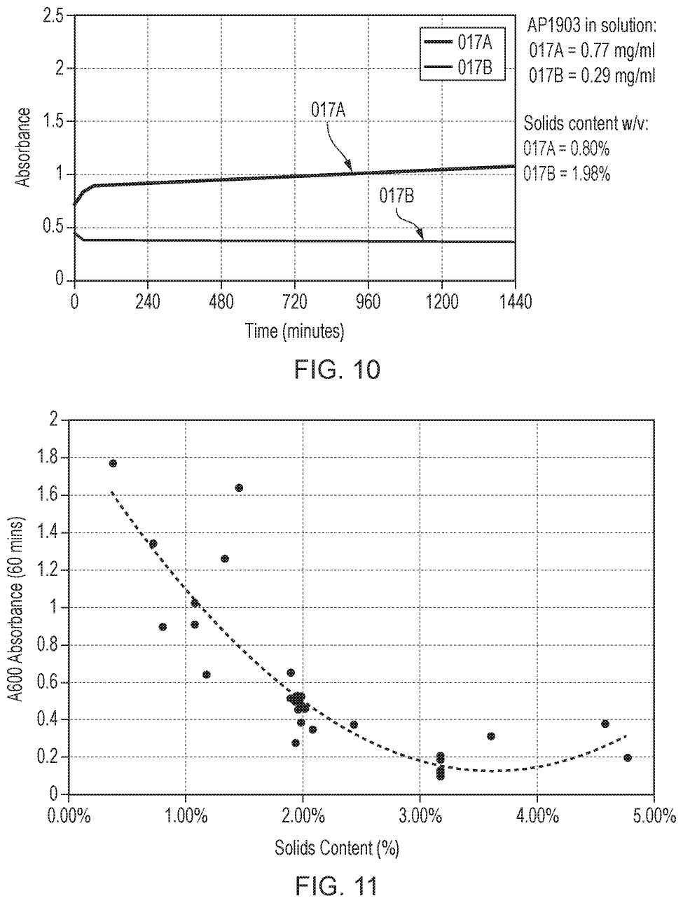

The dissolution of the formulations in DI water was tested at an AP1903 concentration of 0.88 mg/mL over 24 hours. No differences in the A600 values over time were observed between the formulations (FIG. 8), indicating that chilling of the formulations made little difference to the dissolution performance.

Albumin Ratio Effects

In these formulations, the increased solubility of AP1903 relies on its binding to rHA. The nature of that binding, and the amount of AP1903 that can be bound was investigated by changing the molar ratios of AP1903: rHA. In this series of experiments lower ratios were tested (Table 5).

TABLE 5
Batches prepared with reduced AP1903:rHA molar ratios to test the effect on
product performance

| | | | | Extra | | | | | |
|---|---|---|---|---|---|---|---|---|---|
| | | | | EtOH | | Liquid | | | |
| | | Batch | AP1903 | required | | feed | inlet | Outlet | Atomisation |
| Experimental | | size | Loading | in feed | AP1903:rHA | rate | temp | temp | Pressure |
| Rationale | Batch | (mg) | wt % | (ml) | Molar Ratio | (g/min) | (° C.) | (° C.) | (bar) |
| Reducing | 01 SA | 1300 | 7.7 | 0.0 | 4:1 | 2.0 | 108 | 65 | 3.0 |
| 1903: | 015B | 1900 | 5.3 | 0.0 | 3:1 | 2.0 | 104 | 65 | 3.0 |
| ratio | 015C | 2500 | 4.0 | 0.0 | 2:1 | 2.0 | 104 | 65 | 3.0 |

All batches were successfully prepared, and recovered as free flowing powders. Yields ranged from 52 to 67% and correlated with the batch size.

The loading of AP1903 was measured in the spray dried powders from batches 015A, B and C using the solid phase extraction method, and loadings of 97, 101 and 103% vs the expected nominal loadings were obtained respectively.

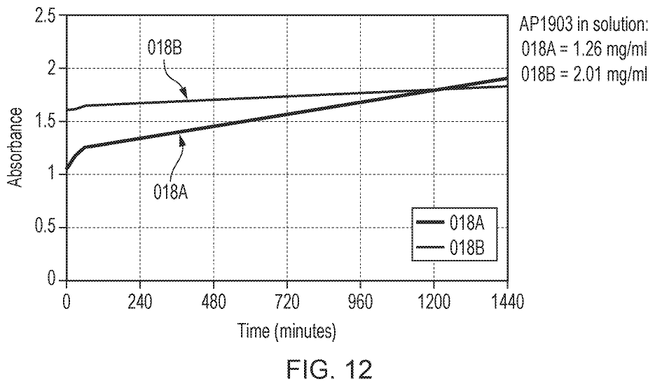

The dissolution of the formulations in DI water was tested, at AP1903 concentrations of 0.39, 0.27 and 0.21 mg/ml for formulations 015A, B and C respectively, over 24 hours. The A600 values obtained decrease with decreasing molar ratio of AP1093:rHA, although the differences are small and most likely arise from the differences in the AP1093 concentrations tested. All traces show similar solution stability over 24 hours (FIG. 9).

Feed Solution Volume

An alternative route to changing the formation of the AP1903:rHA complex is to adjust the concentration of the components in the feed solution. A higher concentration should lead to a higher rate of collisions between AP1903 and rHA, and therefore a more efficient binding process.

Two batches were prepared with a low volume/high concentration and high volume low concentration (Table 6). Both were spray dried successfully with yields of 35%. These were lower than those obtained previously.

TABLE 6
Batches prepared with increased and reduced feed solution volumes to test the
effect of feed solution concentration on product performance

| | | | | Extra | | | | | |
|---|---|---|---|---|---|---|---|---|---|
| | | | | EtOH | | Liquid | | | |
| | | Batch | AP1903 | required | | feed | inlet | Outlet | Atomisation |
| Experimental | | size | Loading | in feed | AP1903:rHA | rate | temp | temp | Pressure |
| Rationale | Batch | (mg) | wt % | (ml) | Molar Ratio | (g/min) | (° C.) | (° C.) | (bar) |
| Total feed | 017A | 700 | 14.3 | 0.0 | 8:1 | 2.0 | 106 | 65 | 3.0 |
| solution vol 81 | | | | | | | | | |
| ml | | | | | | | | | |
| Total feed | 017B | 700 | 14.3 | 1.5 | 8:1 | 2.0 | 106 | 65 | 3.0 |
| solution vol 33 | | | | | | | | | |
| ml | | | | | | | | | |

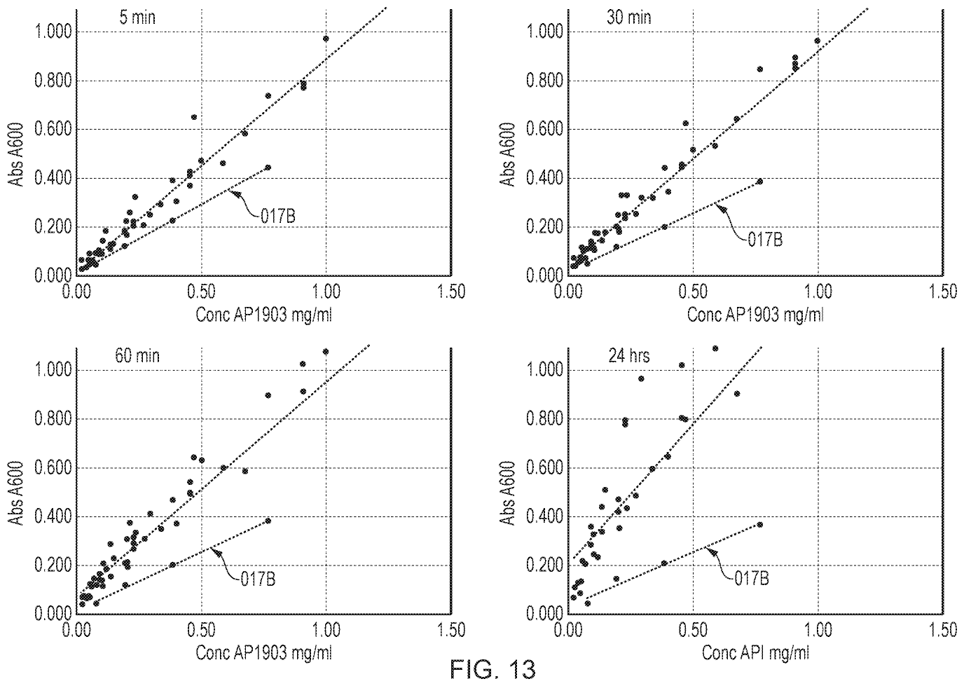

The dissolution of the formulations in DI water was tested at AP1903 concentrations of 0.77 and 0.29 mg/mL for formulations 017A and B respectively over 24 hours. The A600 values obtained were lower for the 017B formulation prepared at the higher concentration, and this formulation appeared more stable over the 24 hour time period (FIG. 10).

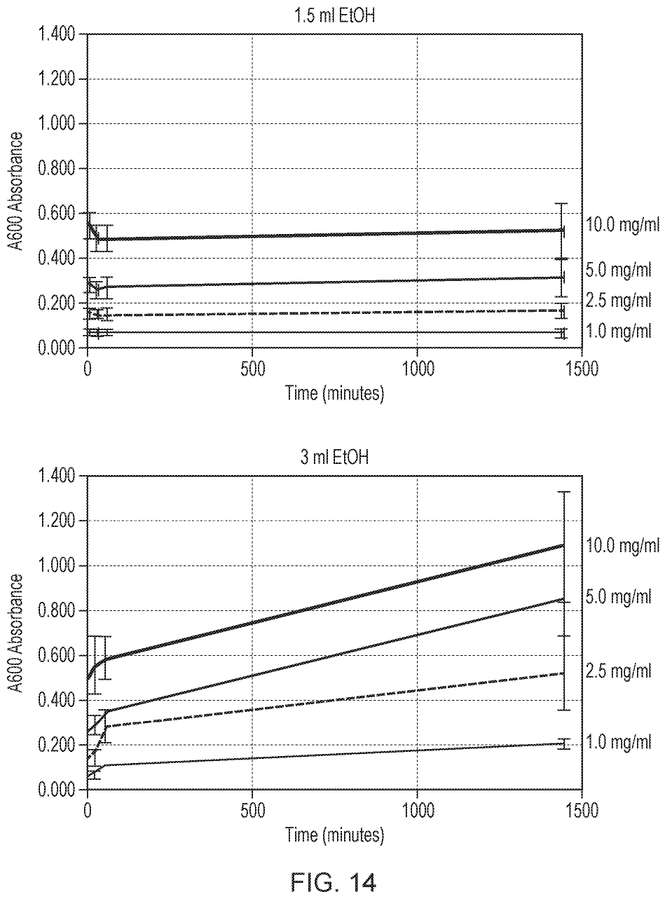

Considering data from all the batches produced, if the solids content (w/v %) in the spray drying feed solution is plotted against A600 at 60 min, the effect of the feed solution concentration can be clearly seen (FIG. 11).

A concentration of 3 w/v % solids or higher in the feed solution is the threshold above which a good A600 performance is observed.

The loading of AP1903 was measured in the spray dried powders from batches 017A and B using the solid phase extraction method. Loadings of 100 and 37% vs the expected nominal loadings were obtained respectively. The solution prepared at the higher concentration gave a much lower loading that was expected. Clearly the reduction in volume, and concomitant reduction in ethanol in the feed solution resulted in less AP1903 being incorporated into the spray dried powder than expected. This was investigated further, and the location of the losses in the process identified through sampling of the solutions and process vessels at the various steps throughout the preparation. This work was undertaken on the repeat formulations 022A, C and D, and is detailed in Example 3.

API Loading

To test the effects of higher AP1903 loadings, two further batches were made with increased loadings of AP1903 (Table 7). Batch 018A contained approximately double the loading used in other batches. Batch 018B contained the maximum loading that could be achieved within the current feed solution preparation constraints (the maximum ratio of ethanol to water without damage to the rHA).

TABLE 7
Batches prepared with increased AP1903 loadings to test the effect on product
performance

| | | | | Extra | | | | | |
|---|---|---|---|---|---|---|---|---|---|
| | | | | EtOH | | Liquid | | | |
| | | Batch | AP1903 | required | | feed | inlet | Outlet | Atomisation |
| Experimental | | size | Loading | in feed | AP1903:rHA | rate | temp | temp | Pressure |
| Rationale | Batch | (mg) | wt % | (ml) | Molar Ratio | (g/min) | (° C.) | (° C.) | (bar) |
| increased | 018A | 700 | 25 | 0.0 | 16:1 | 2.0 | 101 | 65 | 3.0 |
| Loading | | | | | | | | | |
| Max loading | 018B | 800 | 40 | 0.0 | 31:1 | 2.0 | 102 | 65 | 3.0 |
| achievable | | | | | | | | | |
| within | | | | | | | | | |
| constraints of | | | | | | | | | |
| EtOH in feed | | | | | | | | | |

Both batches were prepared and spray dried successfully with yields of 40 and 30% for 018A and 018B respectively. The loading of AP1903 was measured in the spray dried powders and loadings of 88 and 80% vs the expected nominal loadings were obtained.

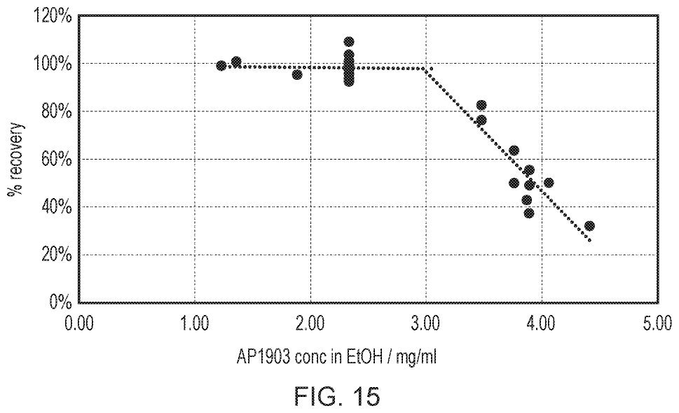

The dissolution of the formulations in DI water was tested at an AP1903 concentrations of 1.26 and 2.01 mg/mL for formulations 018A and B respectively over 24 hours. The A600 values obtained were high compared to previous formulations, and showed a gradual increase over the 24 hour time period (FIG. 12).

Testing of Lead Formulation—Repeat 0178

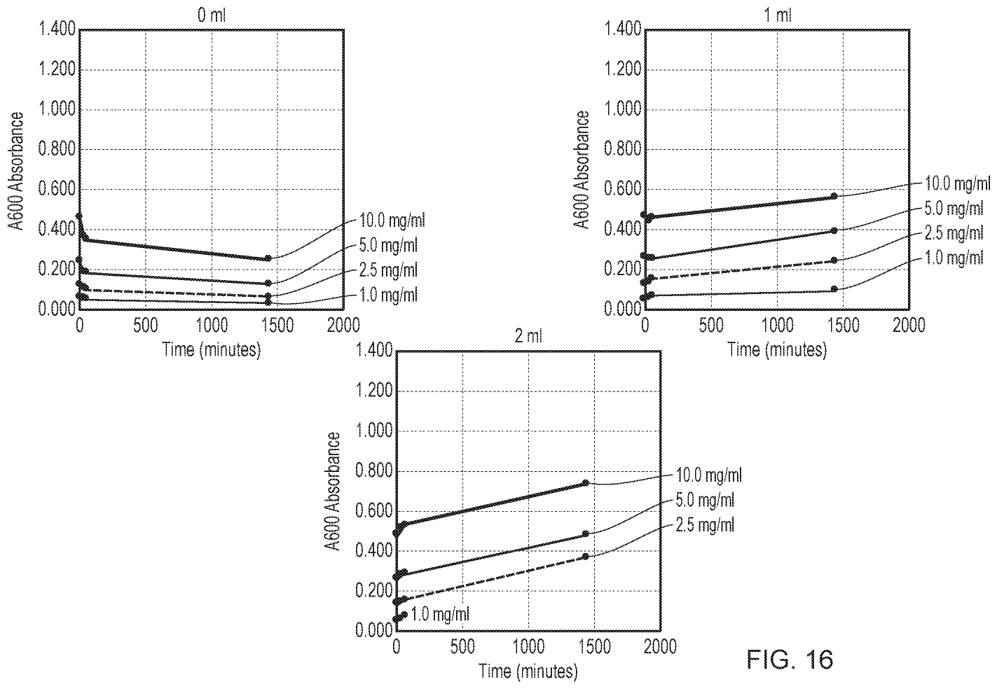

Further analysis of past results was undertaken by plotting A600 vs AP1903 concentration overtime for all formulations prepared (FIG. 13). This showed that for most formulations A600 correlated with API concentration, and most fell on the same line at each timepoint. This showed that the A600 value obtained was primarily controlled by the concentration being tested, irrespective of the formulation or processing methods used. The exception to this was formulation 017B which was seen to deviate from this line, with lower A600 values for a given concentration. This was especially the case at longer timepoints, indicating this formulation was more stable than others overtime.

Considering all available information, 017B was identified as the most promising formulation with low A600 values that were the most stable over time, therefore repeat formulations were made for further testing (Table 8).

It was found during the preparation of these batches that the amount of additional ethanol needed to obtain a visually clear feed solution varied, and was subjective according to the operator preparing the batch. Volumes of 1.5 to 3.0 mL were added, and the batches spray dried. In all cases the batches sprayed well with typical yields.

TABLE 8
Batches prepared as repeats of batch 017B

| | | | | Extra | | | | | |
|---|---|---|---|---|---|---|---|---|---|
| | | | | EtOH | | Liquid | | | |
| | | Batch | AP1903 | required | | feed | inlet | Outlet | Atomisation |
| Experimental | | size | Loading | in feed | AP1903:rHA | rate | temp | temp | Pressure |
| Rationale | Batch | (mg) | wt % | (ml) | Molar Ratio | (g/min) | (° C.) | (° C.) | (bar) |
| Repeats of | 019A | 700 | 14.3 | 3.0 | 8:1 | 2.0 | 106 | 65 | 3.0 |
| to obtain a | | | | | | | | | |
| visually ciear | | | | | | | | | |
| feed solution | | | | | | | | | |
| 017B | 021A | 700 | 14.3 | 3.0 | 8:1 | 2.0 | 102 | 65 | 3.0 |
| Different | 021B | 700 | 14.3 | 2.0 | 8:1 | 2.0 | 106 | 65 | 3.0 |
| ethanol | G21C | 700 | 14 3 | 1 5 | 8:1 | 2.0 | 103 | 65 | 3.0 |
| volumes | 021D | 700 | 14.3 | 1.6 | 8:1 | 2.0 | 100 | 65 | 3.0 |
| were added | 021E | 700 | 14.3 | 1.5 | 8:1 | 2.0 | 101 | 65 | 3.0 |

The loading of AP1903 was measured in all the spray dried powders from these batches, with loadings between 42 and 82% of the expected nominal loadings being obtained. The measured loadings appeared to correlate with the volume of ethanol added back into the feed solution, with high volumes leading to higher loadings and vice versa.

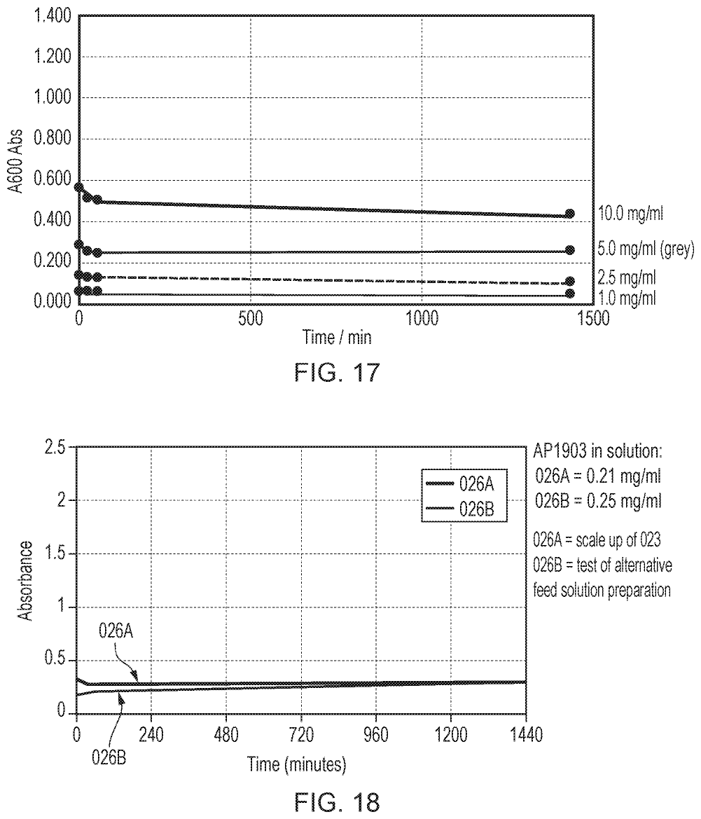

A600 measurements were also taken, and were also found to correlate with the ethanol addition, with high volumes of added ethanol in the feed leading to high A600 measurements (FIG. 14).

Conclusions

Taking a lead concept formulation from the preliminary work, a range of processing parameters and formulation compositions were tested around this to investigate the effects on the formulation performance. Important attributes of the final formulation that were tested include the A600 values and stability of the measurement overtime in DI water, and the AP1903 loading in the formulation.

The key aspects of the process that were identified to influence the attributes included the concentration of solids and ethanol content of the feed solution.

Higher concentrations of solids in the spray drying feed solutions were found to result in powders with comparatively lower A600 values in solution, at the same API concentration, and also to improve the stability of the A600 measurements over time. It was thought that this may arise from improved binding of API to rHA in the feed solution, due to the higher concentrations.

It was also found that the addition of larger volumes of ethanol to the feed solution, in order to produce a visually clear solution, led to formulations with higher and less stable A600 results in solution. Conversely, the addition of lower volumes of ethanol led to lower recovered loadings of AP1903 in the formulations. At this stage, the reasons for these observations were not clear, and additional work was undertaken in the next section to investigate this further.

### Example 3—Effect of Ethanol Volume

From the previous work, the volume of ethanol added to the feed solution was clearly having an influence on the dissolution performance. To this point, ethanol was titrated back into the formulation to produce a visually clear solution. The amount required was found to vary from batch to batch. In this study, a range of volumes of ethanol were added systematically to investigate the effects of the added volume.

Methods

Feed Solution Preparation

AP1903 was dissolved in ethanol at a concentration of 40 mg/mL. The required volume of this solution to give the desired mass of AP1903 was added dropwise to a 50:50 solution of ethanol: HPLC grade water with stirring. A dilute solution of rHA was prepared and the AP1903 solution was added dropwise with stirring. Further ethanol was added into the solution as indicated in Table 9.

Spray Drying

All batches were spray dried using a Buchi B-290 as described in the corresponding section of Example 1 Methods. The spray drying conditions used are listed in Table 9.

Loading Measurements

Loading measurements were undertaken as described in the corresponding section of Example 1 Methods.

A600 Measurements

The absorption of the samples at 600 nm was measured as described in the corresponding section of Example 1 Methods.

Results

To test the effects of the volume of ethanol added to the feed solution, four batches were made with fixed volumes added ranging from 0-2 mL (Table 9).

In contrast to the preparation of the earlier feed solutions, a fixed volume of ethanol was added and the batch spray dried from the feed solution as existed in that state. All batches were found to spray successfully, and batch yields ranging from 22 to 60% were obtained.

TABLE 9
Batches prepared to test the addition of ethanol to the feed solution

| | | | | Extra | | | | | |
|---|---|---|---|---|---|---|---|---|---|
| | | | | EtOH | | Liquid | | | |
| | | Batch | AP1903 | required | | feed | inlet | Outlet | Atomisation |
| Experimental | | size | Loading | in feed | AP1903:rHA | rate | temp | temp | Pressure |
| Rationale | Batch | (mg) | wt % | (ml) | Molar Ratio | (g/min) | (° C.) | (° C.) | (bar) |
| Testing | 022A | 700 | 7.7 | 0.0 | 8:1 | 2.0 | 98 | 65 | 3.0 |
| addition of a | 022B | 700 | 7.7 | 1 0 | 8:1 | 2.0 | 98 | 65 | 3.0 |
| range of | 022C | 700 | 7.7 | 2.0 | 8:1 | 2.0 | 102 | 65 | 3.0 |
| EtOH | | | | | | | | | |
| volumes to | | | | | | | | | |
| feed solution | | | | | | | | | |
| Repeat 022C | 022D | 700 | 7,7 | 2 0 | 8:1 | 2.0 | 94 | 65 | 3.0 |

The loadings of AP1903 were measured in the batches and found to range from 32 to 65%. These again correlated with the volume of addition of ethanol, with high volumes giving high loadings, and low volumes giving low loadings. However, all loadings were lower than anticipated, and it was clear that some AP1903 was being lost during processing. Samples were taken from solutions, vessels and equipment at multiple steps during the processing of batch 022C to investigate the location of any remaining AP1903. It was found that the majority of AP1903 which was not found in the final formulation could be found in the residue on the walls of the feed solution container after spray drying (Table 10).

TABLE 10
Mass balance of AP1903 obtained during the processing
of formulation 022C. AP1903 was recovered from
the formulation and feed solution vessel.

| Loading in | Recovered from | |
|---|---|---|
| Formulation | Feed Vessel | Total |
| 63.4% | 39.7% | 103.1% |

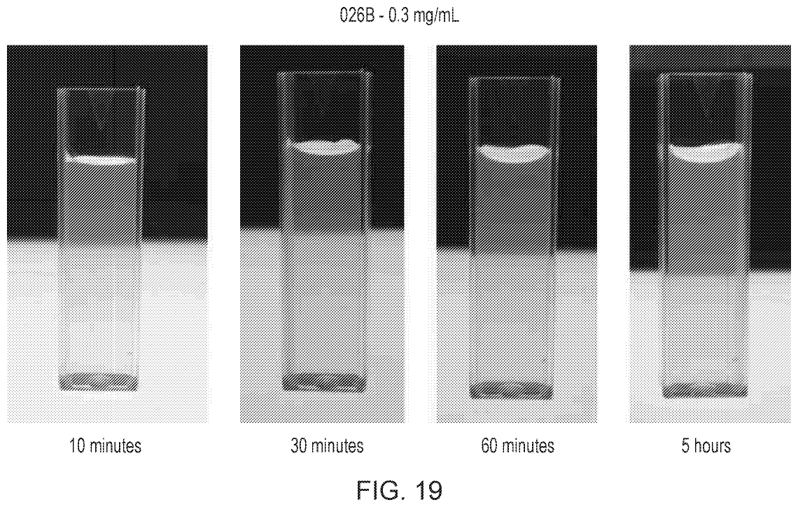

This indicated that the AP1903 had not remained in solution, and thus was not spray dried. This would further suggest that this material was not bound to the rHA during the feed solution preparation stage. Considering the feed solution, it is the ethanol present in this solution which keeps the AP1903 solubilised such that it can bind to the rHA during processing. It would therefore follow that there was a maximum concentration of AP1903 in ethanol in the feed solution beyond which all the AP1903 would not remain dissolved, and would precipitate out. Plotting measured loading against this concentration suggested the threshold lay around 3 mg AP1903 per mL ethanol (FIG. 15).

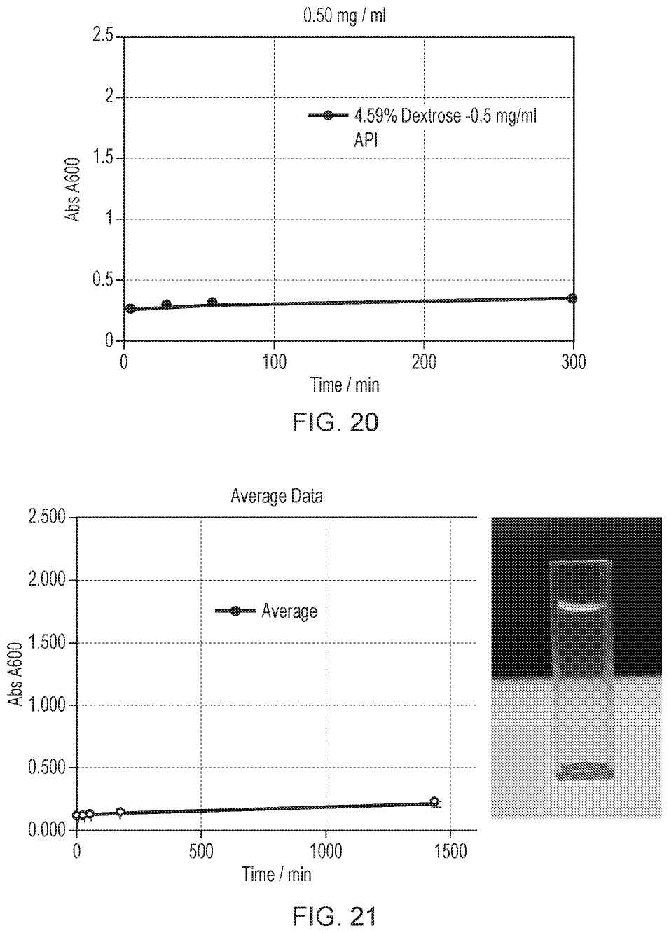

The dissolution of the formulations was measured in DI water, and the A600 values were again found to correlate with the volume of ethanol added, with lower volumes giving better performance with lower A600 values and better stability (FIG. 16).

Conclusions

Taking all the results in information gathered on the lead formulation together, a more complete understanding of the system could be obtained. At the highest level, a spray dried material was being prepared which consisted of the ethanol soluble AP1903 binding to water soluble rHA. Considering the binding in more depth, there were a finite number of AP1903 molecules that can bind to a molecule of rHA, and once all the rHA molecules have been saturated, no further AP1903 can be bound. What then happened to any excess AP1903 present depended on the amount of ethanol present in the feed solution. If a relatively high volume of ethanol was present, the AP1903 remained in solution and was spray dried as free drug. A 100% recovery of API was then achieved when the loading in the spray dried formulation was measured. But the A600 values for the reconstituted solution were high, and solution stability was poor, as the free drug was not solubilised. However, if the volume of ethanol in the feed solution was relatively low, the excess AP1903 precipitated out, and was not spray dried. The spray dried product then contained only AP1903 that was bound to rHA. This resulted in lower A600 values for the reconstituted solution, and good solution stability. But there was poor recovery of drug when the loading was measured. Examples of both of these scenarios can be seen in the previous examples.

This analysis suggested that to get the best performing product, free insoluble AP1903 arising as a result of spray drying excess AP1903 dissolved in ethanol, should be avoided. This was not present in any of the batches which performed well, but which were made with low ethanol volumes, and gave low loading due to AP1903 losses though precipitation. This would suggest the 8:1 molar ratio of AP1903:rHA was too high and only approximately 40% of the AP1903 was being bound to the rHA. Using this to back-calculate a molar ratio for complete binding suggests a molar ratio of 3:1 might be more appropriate to achieve a product that performs well with a 100% AP1903 recovery.

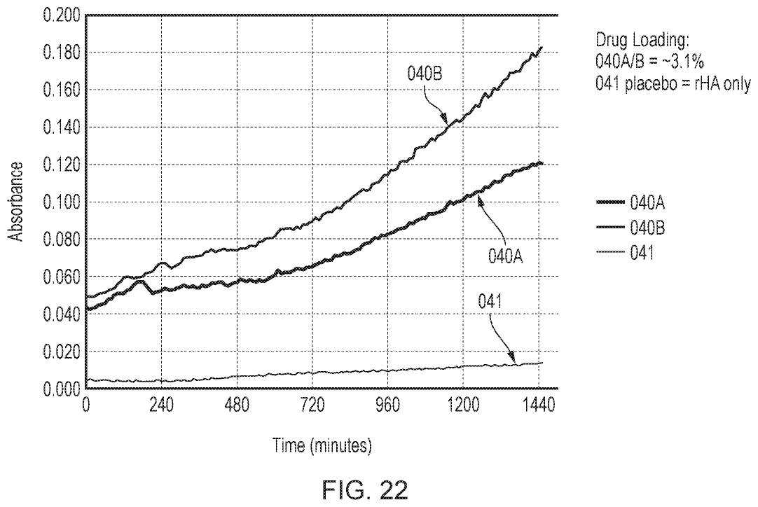

In order to test this a further batch (023) was made in an identical manner to those described in section 5.2, but with the AP1903:rHA molar ratio reduced to 3:1. This produced a product with a measured loading of 98%, and a good, stable A600 performance (FIG. 17).

### Example 4—Scale Up

With a good understanding of the batch preparation process, and a lead candidate formulation identified, increasing the scale of the batch preparation with the lead formulation was desirable to understand if the formulations could be made on a larger scale.

An alternative feed solution preparation method was developed which was thought to be more suitable for larger scale production. The sample prepared with this alternative method also had an increased solids content as this appeared to be a contributing factor for better reconstitution performance, based on information from the process and formulation optimisation studies.

An additional dissolution test was added, where the formulations were dissolved back into a 5 w/v % dextrose solution (D5W), to give an isotonic solution that would be suitable for IV administration.

Methods

Feed Solution Preparation

Standard Feed Solution Preparation:

AP1903 was dissolved in ethanol at a concentration of 40 mg/mL. The required volume of this solution to give the desired mass of AP1903 was added dropwise to a 50:50 solution of ethanol: HPLC grade water with stirring. A dilute solution of rHA was prepared and the AP1903 solution was added dropwise with stirring. During this addition, the solution initially went from clear to opaque, as the API precipitated out of the aqueous solution. As further API solution was added, and hence the ethanol content of the solution increased, the solution gradually clarified, as the API re-dissolved.

Alternative Feed Solution Preparation:

AP1903 was dissolved in ethanol at a concentration of 2.5 mg/mL. The entirety of this solution was added dropwise to a dilute solution of rHA with stirring. As above, the solution went from clear to opaque during this addition which indicated the API precipitating out of the aqueous solution. However, the solution gradually clarified as further API solution was added and the API re-dissolved.

Spray Drying

All batches were spray dried using a Buchi B-290 spray dryer as described in the corresponding section of Example 1 Methods. The spray drying conditions used are listed Table 11.

Loading Measurements

Loading measurements were undertaken as described in the corresponding section of Example 1 Methods.

A600 Measurements

The absorption of the samples at 600 nm was measured using a Shimadzu UV-Vis. Samples were reconstituted in both DI water and a water/D5W system for comparison. The DI water reconstitution method is described in the corresponding section of Example 1 Methods. Samples reconstituted in the water/D5W system were prepared by first dissolving the spray dried powder in HPLC grade water at a concentration of 200 mg/mL. Once dissolved, this solution was transferred into D5W using a needle and syringe to give the final concentration shown. Samples were made up in 2 mL polystyrene cuvettes, and the absorbance at 600 nm recorded at time intervals up to 24 hours post dissolution. Optical images of the cuvettes were also taken as a record of the visual appearance of the solutions.

Results

To test the effects of scaling up the lead formulation, several batches were made comparing the large and small scale, and an alternative feed solution preparation method was developed which was thought to be more suitable to use on a larger scale (Table 11).

All batches processed well, and yields of 87% were achieved for the two larger scale batches, and 76% for the smaller batch 026E.

TABLE 11
Batches prepared to test the scaling up of the lead formulation

| | | | | Extra | | | | | |
|---|---|---|---|---|---|---|---|---|---|
| | | | | EtOH | | Liquid | | | |
| | | Batch | AP1903 | required | | feed | inlet | Outlet | Atomisation |
| Experimental | | size | Loading | in feed | AP1903:rHA | rate | temp | temp | Pressure |
| Rationale | Batch | (mg) | wt % | (ml) | Molar Ratio | (g/min) | (° C.) | (° C.) | (bar) |
| Large scale. | 026A | 3700 | 5.8 | 0.0 | 3:1 | 2.0 | 108 | 65 | 3.0 |
| Standard feed | | | | | | | | | |
| solution prep | | | | | | | | | |
| Large scale, | 026B | 4100 | 6.1 | 0.0 | 3:1 | 2.0 | 104 | 65 | 3.0 |
| Alternative | | | | | | | | | |
| feed solution | | | | | | | | | |
| prep | | | | | | | | | |
| Repeat small | 026E | 600 | 5.6 | 0.0 | 3:1 | 2.0 | 104 | 65 | 3.0 |
| scale hatch | | | | | | | | | |
| standard feed | | | | | | | | | |
| solution prep | | | | | | | | | |

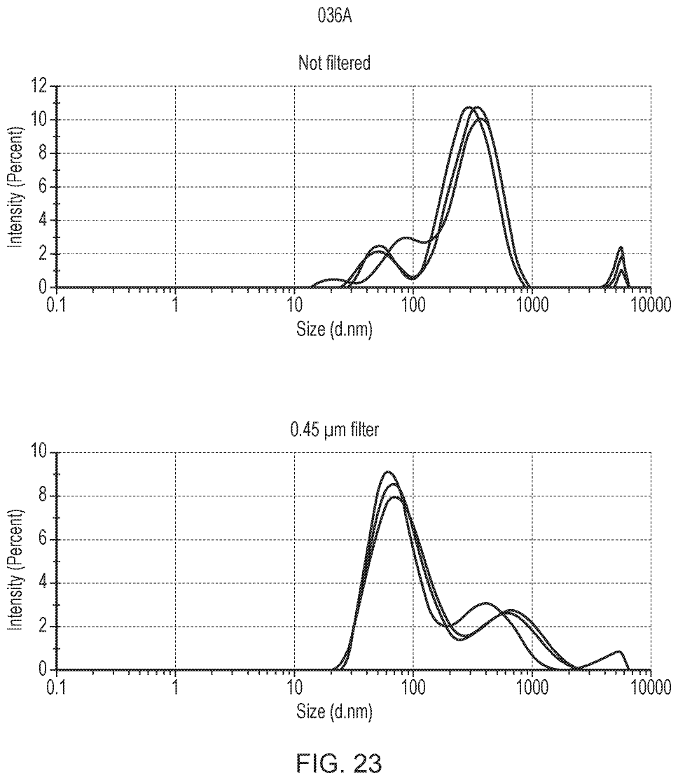

The dissolution of the formulations in DI water was tested at AP1903 concentrations of 0.21 and 0.25 mg/mL for formulations 026A and B respectively over 24 hours (FIG. 18). The A600 values obtained were low and showed good stability over 24 hours. No difference could be observed between the two formulations, indicating the feed solution preparation method has little impact on the A600 values obtained.

The results obtained here for 026B can be directly compared to those shown by the grey trace in FIG. 17 for formulation 023 which was prepared using the same methods, but on a smaller scale. No difference is observed between the two batches in terms of dissolution performance.

D5W was tested in order to find a more relevant dissolution medium that would allow the formulations to be dissolved in an isotonic solution.

The dissolution of the formulation directly into D5W was tested first, but found to be unsuccessful, with the formation of a very hazy, lumpy suspension rather than a solution.

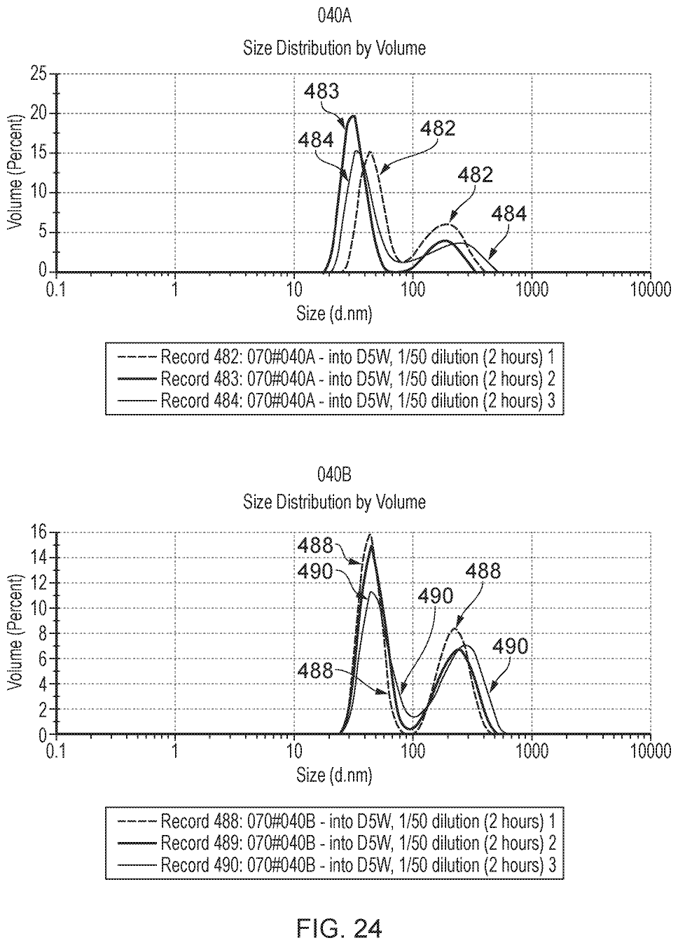

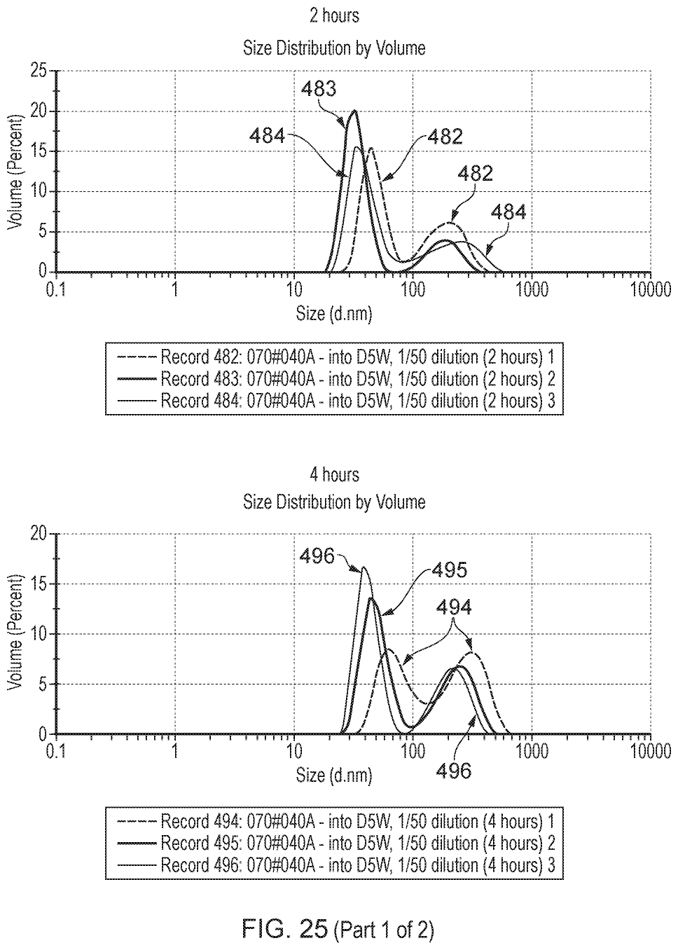

In order to overcome this, an alternative method was devised whereby the formulation was initially dissolved in a small volume of water. This initial solution was then transferred, using a needle and syringe, to a larger volume of D5W to produce a solution that was stable overtime (FIG. 19 and FIG. 20).

Conclusions

Preparation of batches on the 4 g scale was successful, and they were shown to perform similarly to batches prepared on the smaller scale. An alternative solution preparation method more suited to larger scale batches was tested and presented A600 measurements very similar to those produced by the sample made via the standard solution preparation method. However, as sample 026B had an increased solids content, which was previously shown to improve reconstitution, the alternative solution preparation method may have caused slightly higher A600 measurements. The standard solution preparation was therefore carried forward with the lead formulation.

### Example 5—Lead Candidate Testing

In the previous work a lead formulation has been identified, together with the processing parameters for its production and test methods to characterise it. In this section, manufacture of this lead formulation is repeated and analysis of these repeat batches is undertaken.

Methods

Feed Solution Preparation

AP1903 was dissolved in ethanol at a concentration of 7.2 mg/mL. The required volume of this solution to give the desired mass of AP1903 was added dropwise to a 50:50 solution of ethanol: HPLC grade water with stirring. A dilute solution of rHA was prepared and the AP1903 solution was added dropwise with stirring. During this addition, the solution went from clear to opaque then back to clear due to precipitation and re-dissolving of the AP1903.

Spray Drying

All batches were spray dried as described in the corresponding section of Example 1 Methods. The spray drying conditions used are listed in Table 12.

Loading Measurements

Loading measurements were undertaken as described in the corresponding section of Example 1 Methods.

A600 Measurements

The absorption of the samples at 600 nm was measured using a Shimadzu UV-Vis. Samples were prepared by first dissolving the spray dried powder in HPLC grade water at concentration of 200 mg/mL. Once dissolved, this solution was transferred into D5W using a needle and syringe to give the final concentration shown. Samples were made up in glass scintillation vials, aliquots transferred to polystyrene cuvettes and the absorbance at 600 nm recorded at time intervals up to 24 hours post dissolution. Optical images of the cuvettes were also taken as a record of the visual appearance of the solutions.

For comparison the same samples were analysed on a Thermo Scientific Varioskan lux multimode microplate reader. Samples were plated up in a 96 well Maxisorp Nunc-immuno plate, 200 μL in triplicate vs a 200 μL D5W blank in triplicate. UV detection was set to 600 nm on a kinetic loop every 10 minutes for 24 hours (Pulsed shaking for 20 seconds every 2 minutes at 300 rpm).

Zetasizer® and Zeta Potential Measurement

For nanoparticle size analysis samples were analysed in in D5W using Malvern Instruments Zetasizer®. Samples were analysed taking the concentration used in the A600 measurements, and diluting between 1:50 and 1:100 with further D5W based on visual observation of the sample. Sufficient dilution rendered the sample visually clear. For analysis samples were contained in a 2 mL polystyrene cuvette. The Z-average size and PDI were measured.

Zetapotential measurements were also conducted on a Malvern Instruments Zetasizer®, using solution prepared at 0.3 mg/mL AP1903. Samples were analysed at pH 7.0.

Nanosight Imaging

Nanoparticle Tracking Analysis (NTA) was undertaken with a Malvern Nanosight LM14. The Nanosight systems pass a laser beam through the sample chamber, where the particles in suspension in the path of the beam scatter light that is visualised by a 20× magnification video microscope. Sequential (30 frames p/second) files of the scattered light can then be analysed and a hydrodynamic radius calculated using the Stokes-Einstein equation.

Analysis was performed at 1 in 10000 dilution of the neat suspension, collecting data over 5×60 second captures using conventional scattering analysis.

rHA SEC Analysis

Size exclusion chromatography was performed on an Agilent 1100 series HPLC employing a TsK gel G3000SWXL (300×7.8 mm I.d., 5 μm) column, an isocratic flow of phosphate buffer (pH 7) at 1 mL/min with UV detection at 210 nm.

Results

Initially three repeat batches were prepared to test the reproducibility of the preparation of the lead formulation (Table 12). The three batches were prepared and spray dried successfully with yields of 74, 75 and 78%.

The loading of AP1903 was measured in the spray dried powders from the repeat batches using the solid phase extraction method, and found to be highly reproducible (Table 13).

TABLE 12
Batches prepared to test the reproducibility of the manufacturing of the lead
formulation

| | | | | Extra | | | | | |
|---|---|---|---|---|---|---|---|---|---|
| | | | | EtOH | | Liquid | | | |
| | | Batch | AP1903 | required | | feed | inlet | Outlet | Atomisation |
| Experimental | | size | Loading | in feed | AP1903:rHA | rate | temp | temp | Pressure |
| Rationale | Batch | (mg) | wt % | (ml) | Molar Ratio | (g/min) | (° C.) | (° C.) | (bar) |
| Repeat batches | 036A | 600 | 6 1 | 0 | 3:1 | 2.0 | 121 | 65 | 3.0 |
| of lead | 036B | 600 | 6.1 | 0 | 3:1 | 2.0 | 127 | 65 | 3.0 |
| formulation | 0360 | 600 | 6 1 | 0 | 3:1 | 2.0 | 125 | 65 | 3.0 |
| | 040A | 1100 | 6.1 | 0 | 3:1 | 2.0 | 95 | 65 | 3.0 |
| | 040B | 1100 | 6.1 | 0 | 3:1 | 2.0 | 90 | 65 | 3.0 |
| Placebo | 041 | 600 | n/a | 0 | n/a | 2.0 | 95 | 65 | 3.0 |

TABLE 13
Measured AP1903 loadings for three
repeat batches of lead formulation

| | Measured |
|---|---|
| Formulation | Loading |
| 036A | 102.8 ± 0.5 |
| 0368 | 102.8 ± 1.0 |
| 036C | 100.8 ± 0.6 |

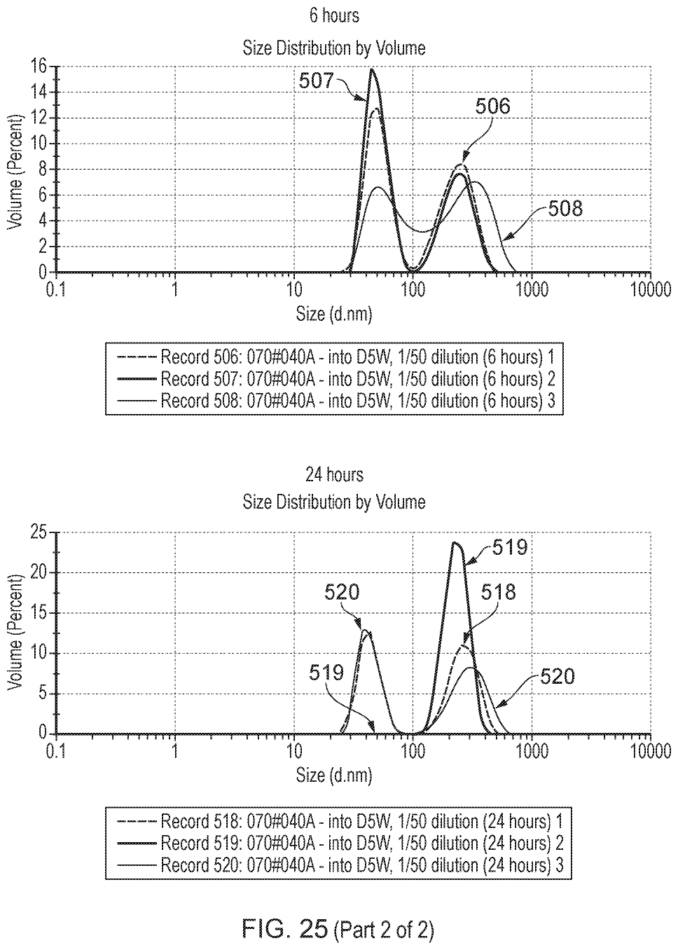

The dissolution of the three formulations in D5W was tested at an AP1903 concentration of 0.30 mg/mL over 24 hours (FIG. 21). The A600 values obtained were low and showed good stability over 24 hours. No difference could be observed between the three repeat formulations, indicating good reproducibility across the three batches.

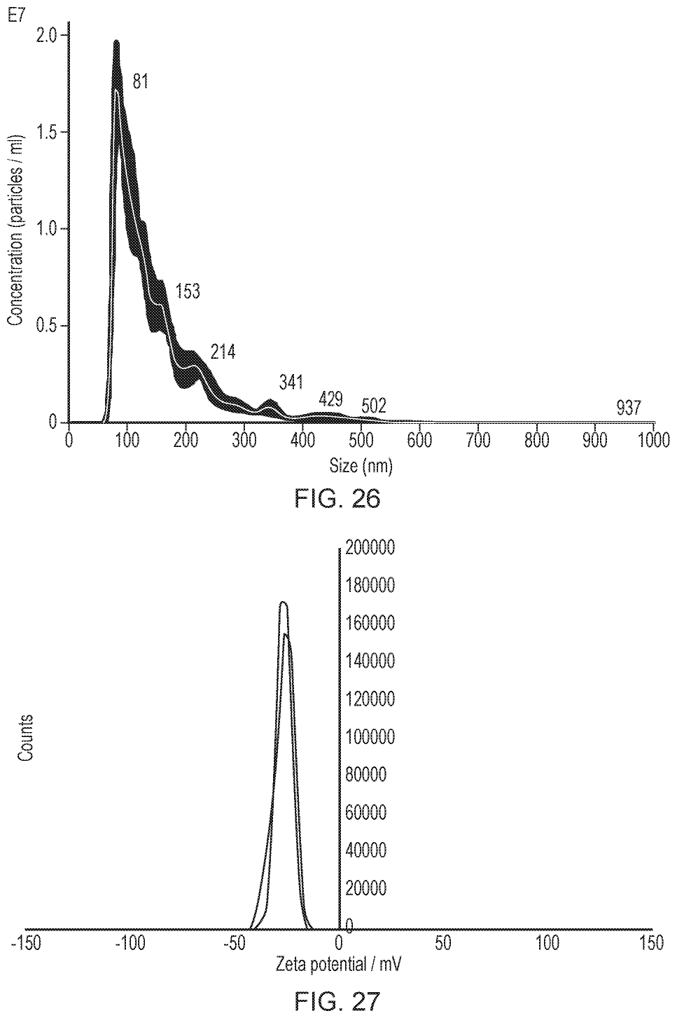

Further repeat batches were prepared, together with a placebo taking rHA alone through the process. The dissolution of these was also studied over 24 hours using a plate reader method which allowed more frequent analysis with smaller timesteps. Both 040A and B performed similarly, with the placebo showing a much lower value as might be expected as the nanoparticle structures seen in the formulation will not be formed in the absence of AP1903 (FIG. 22).

The visual appearance of the solutions formed when the formulations are reconstituted suggested the formation of nanoparticles. This was investigated using several techniques for the investigation of nanoparticle size including dynamic light scattering (DLS) and imaging techniques.

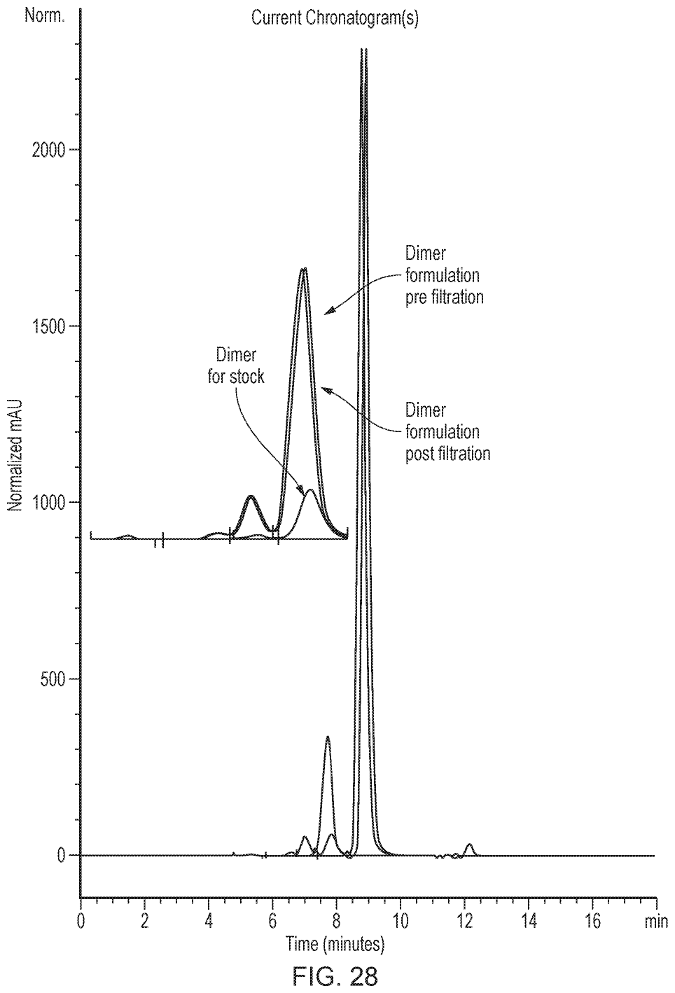

DLS analysis by Zetasizer® was undertaken on the samples prepared for A600 determination, as was done previously in Example 1 Results. However, the AP1903 concentrations that were solubilised here were higher than those previously obtained, and the analysis was found to be highly sensitive to the sample concentration being analysed. Peaks corresponding to large particles >6000 nm in size were found, and these remained after the sample was filtered through a 0.45 μm (450 nm) filter (FIG. 23).

Particles greater than 0.45 μm should not have been present in the solution after filtration, therefore the fact that these were still detected indicates that the peaks may be artefacts. Following discussions with particle size instrument manufacturers (Malvern Instruments, Sympatec), and particle sizing experts at The University of Nottingham, it was suggested that the concentrations of solutions being analysed were too high, and much more dilute solutions should be used. The DLS method is based on laser scattering by a single particle to determine it's size, and therefore, concentrations should be used that are sufficiently low that multiple scattering events are avoided.

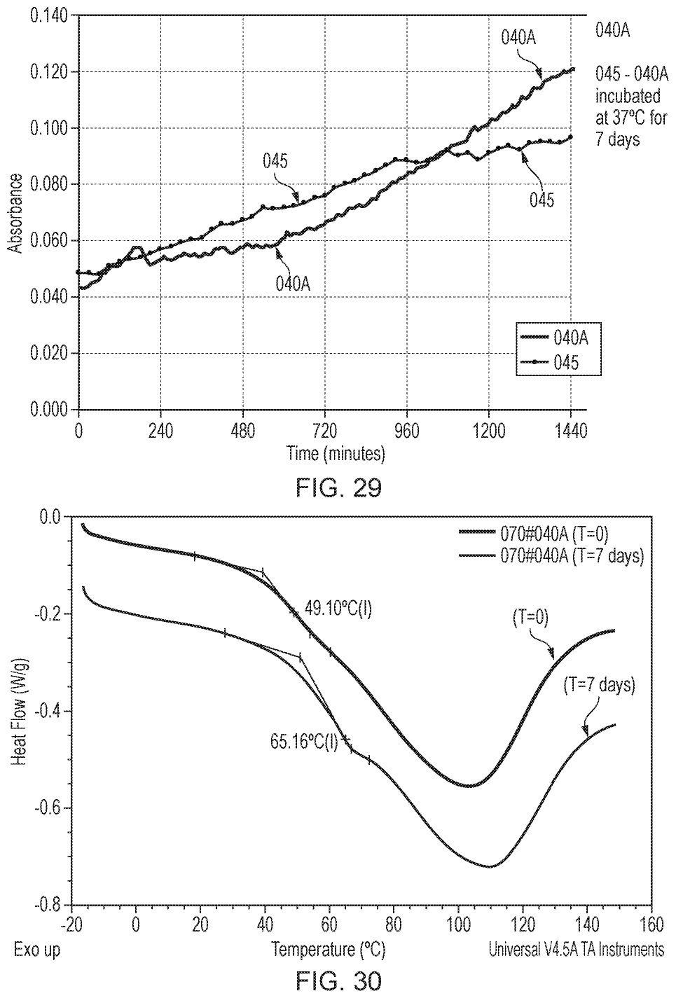

Modifying the analysis method, it was found that 1:50-1:100 dilutions of the standard dissolution solution in D5W gave reproducible measurements, showing a bimodal distribution of particles with peaks centred at 45 and 200 nm (FIG. 24).

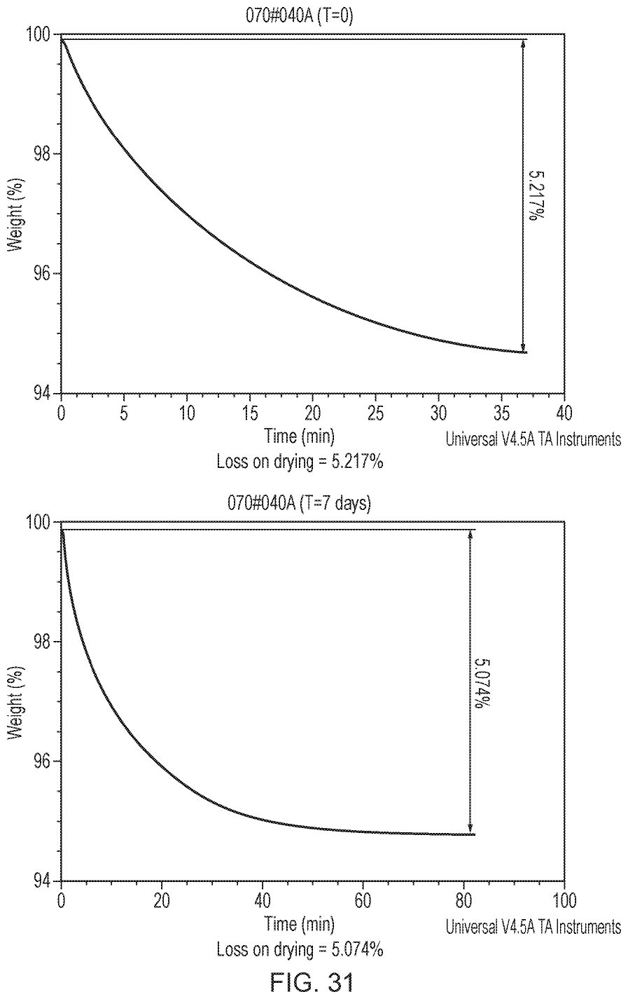

Studying the evolution of the traces over time, it was observed that both peaks remained for 24 hours, although the relative intensities were seen to vary (FIG. 25). This variation may be indicative of the nanoparticles being transient in nature, and therefore variability in the measurements can be expected.

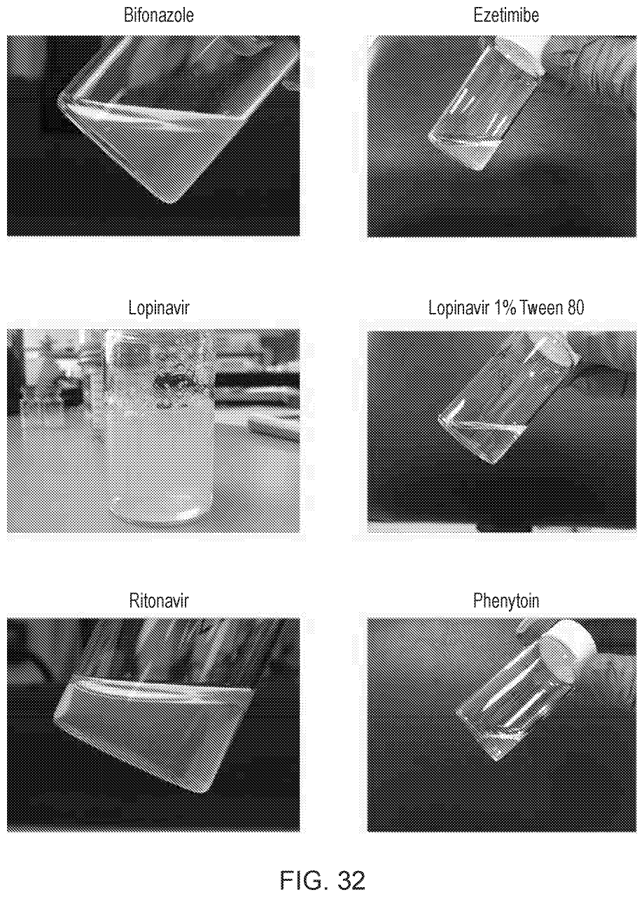

Further analysis was undertaken using a Malvern NanoSight instrument. This permits an imaging technique that captures and analyses images of the nanoparticles directly. This created an average trace for n=5 imaging runs of sample 036B and presents the data as an average+/−1 SD (FIG. 26). This again showed a multi modal distribution with the main peaks at 81, 153 and 214 nm. These data broadly correlate with the Zetasizer® data that were also obtained.

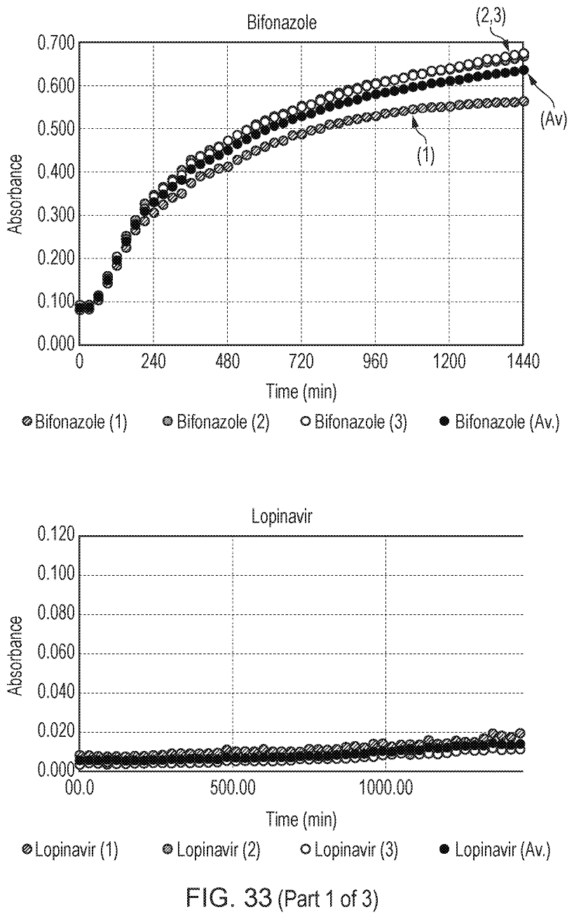

The zeta potential of sample 036B was measured to obtain an idea of the nanoparticles in solution. The average value obtained over n=3 analyses was −28.1*4.3 mV at pH 7, indicating the nanoparticles have moderate stability against flocculation or aggregation (FIG. 27).

HPLC analysis of the AP1903 and SEC analysis of the rHA was undertaken on a sample of the formulation dissolved in D5W before and after filtration through a 1.2 μm filter. Before filtration, 100% of the AP1903 was recovered, and the rHA was found to consist of 81% monomer and 17% dimer. The remainder of the rHA being higher order structures. Post filtration the rHA was unchanged, but the AP1903 recovery had decreased to 84% indicating that some material had been lost in the filter (Table 14).

TABLE 14
Analysis ofAP1903 and rHA content in solution pre and
post filtration through 1.2 μm filter

| | Stock | | Pre-filtration | | Post 1.2 filtration | |
|---|---|---|---|---|---|---|
| Component | Monomer | Dimer | Monomer | Dimer | Monomer | Dimer |
| rHA | 97% | 3% | 81% | 17% | 81% | 17% |

| | | | |
|---|---|---|---|
| AP1903 | n/a | 100% | 84% |

Given that the rHA content did not change, it was speculated that the hydrophobic nature of the filter may be pulling the AP1903 out of the rHA complex, and the AP1903 was therefore being lost selectively on the filter. This was confirmed by repeating the experiment, but with an ethanol wash of the filter following the filtration step. A significant proportion of the lost AP1903 was recovered in the ethanol wash indicating that the filter was indeed removing the AP1903 from the rHA complex.

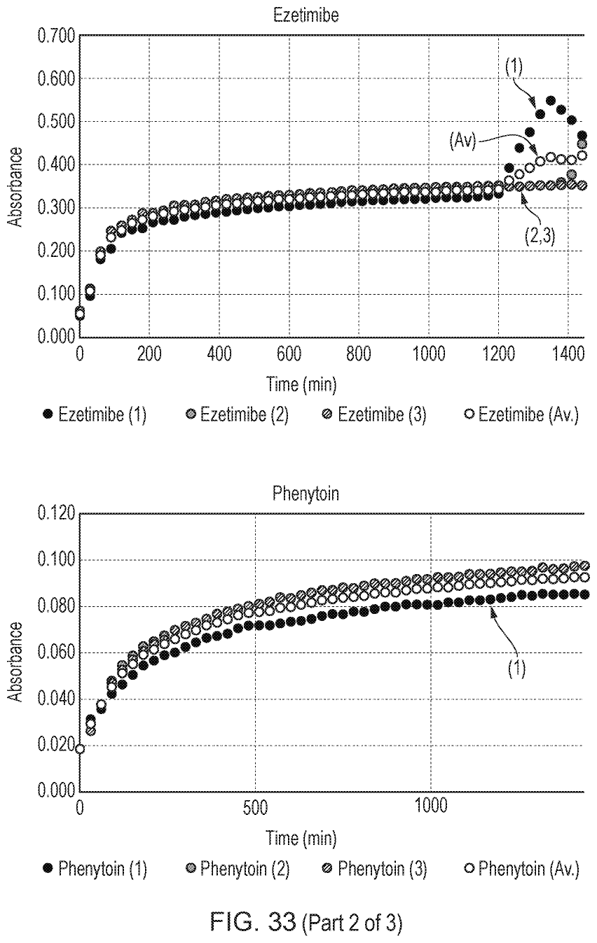

SEC analysis of the rHA component pre and post filtration showed no change, but it was observed that increased dimerization of the rHA was found in the formulations. This may be indicative of the complex formation with the AP1903 causing crosslinking of the rHA (FIG. 28).

Conclusions

The lead formulation had been reproducibly manufactured on five occasions, and analysis of these batches has shown them to perform identically. Analysis of the nanoparticles present in solution has shown them to be of a multimodal size distribution, and they are likely to be transient in nature, as the AP1903 is weakly bound to the rHA, and can be removed from the complex on a hydrophobic filter membrane.

### Example 6—Stability Study

To test the stability of the lead formulation, samples were stored for one week under accelerated temperature conditions of 37° C. and ambient relative humidity. Samples were tested at t=0 and t=7 days for A600 performance of the reconstituted solution, and residual moisture and thermal properties of the powder.

Methods

Loading Measurements

AP1903 loading measurements were undertaken as described in the corresponding section of Example 1 Methods.

A600 Measurements

Samples were prepared by first dissolving the spray dried powder in HPLC grade water at a concentration of 200 mg/mL. Once dissolved, this solution was transferred into D5W using a needle and syringe to give the final concentration shown. Samples were analysed on a Thermo Scientific Varioskan lux multimode microplate reader. Samples were plated up in a 96 well Maxisorp Nunc-immuno plate, 200 μL in triplicate vs a 200 μL D5W blank in triplicate. UV detection was set to 600 nm on a kinetic loop every 10 minutes for 24 hours (Pulsed shaking for 20 seconds every 2 minutes at 300 rpm).

DVS

Loss of mass on drying, by desorption at 0% RH/60° C., was used to determine the residual moisture content of the spray dried powders.

Analysis was performed using a TA Instruments Q5000 SA Dynamic Vapour Sorption (DVS) analyser. For each measurement approximately 20 mg of powder was loaded into a quartz glass sample crucible. The sample was measured using the following method:
1:Equilibrate sample at 25° C./0% relative humidity (RH);
2:Maintain at 0% RH;
3:Ramp temperature at 1° C./min to 60° C.;
4:Hold at 60° C./0% RH until a weight change of <0.010% in 5 minutes is achieved.

The residual moisture was calculated as a percentage weight loss on drying, and data were analysed using TA Instruments Universal Analysis build 4.5.0.5.

DSC

DSC analysis was undertaken using a TA Instruments Q20 MDSC with auto sampler and refrigerated cooling accessory. Approximately 5 mg of sample was run in a T Zero aluminium pan under an N2 flow (50 mL/min). Pans were sealed using a T Zero pan press. The following cycle was used on all samples:
1: Data storage: Off;
2: Equilibrate at −20.00° C.;
3: Isothermal for 5.00 minutes;
4: Data storage: On;
5: Ramp 10.00° C./min to 150.00° C.;
6: Isothermal for 5.00 min;
7: Ramp 10.00° C./min to −20.00° C.;
8: Isothermal for 5.00 min;
9: Ramp 10.00° C./min to 150.00° C.

Data were analysed using TA Instruments Universal Analysis build 4.5.0.5.

Stability Storage

Samples were packaged in screw cap scintillation vials, and the tops wrapped with parafilm. Samples were stored in an incubator at 37° C. for 7 days.

Results

A sample of batch 040A, prepared as part of the lead candidate testing, was packaged and stored at 37° C. for 7 days. The sample was analysed after this time and compared to the sample at t=0, pre-storage.

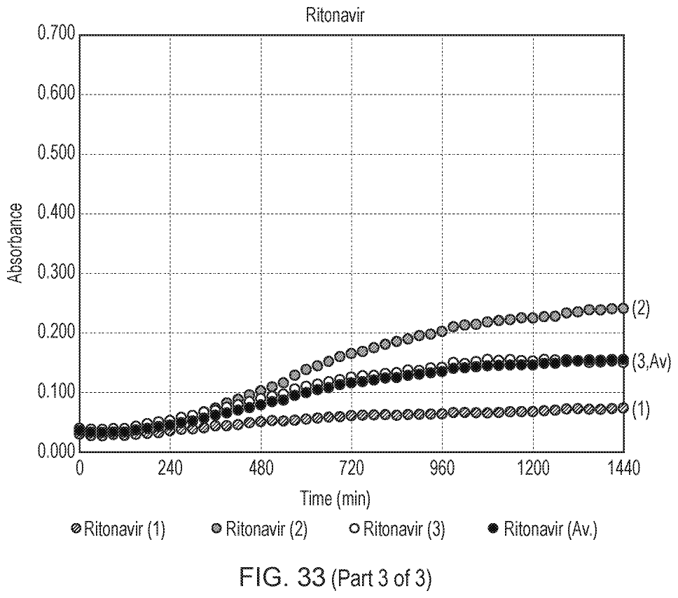

The AP1903 loadings of samples were measured at t=0, 100% (sample 040A) and at t=7 days, 98% (sample 045). No change in AP1903 loading was observed during storage. Dissolution of the sample was tested by A600 measurement over 24 hours (FIG. 29).

The results obtained were low and identical between samples, indicating no change in the dissolution performance of the formulation following storage.

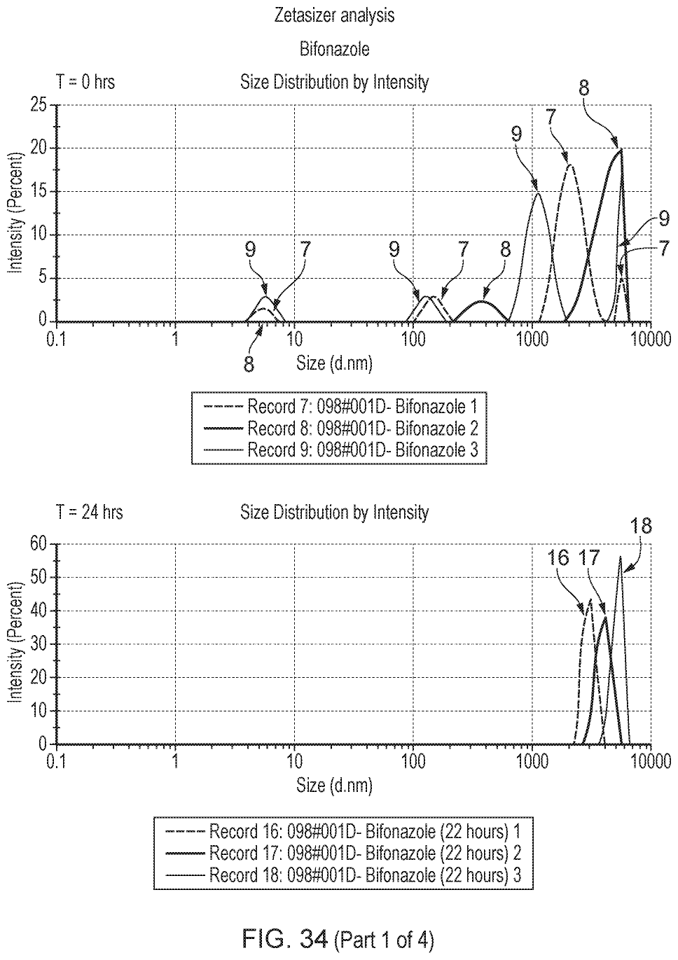

The thermal properties of the formulation before and after storage were measured by DSC, and the first cycle traces were analysed (FIG. 30). Both traces exhibit a glass transition, probably from the trehalose, and a broad endothermic transition arising from the rHA. The traces before and after storage were broadly similar, with the same features present, but an increase in the glass transition temperature of the trehalose was observed; this may be a result of the slight reduction in residual moisture, or possibly from vitrification or aging of the trehalose on storage. The second cycle traces are not shown as no thermal events were observed.

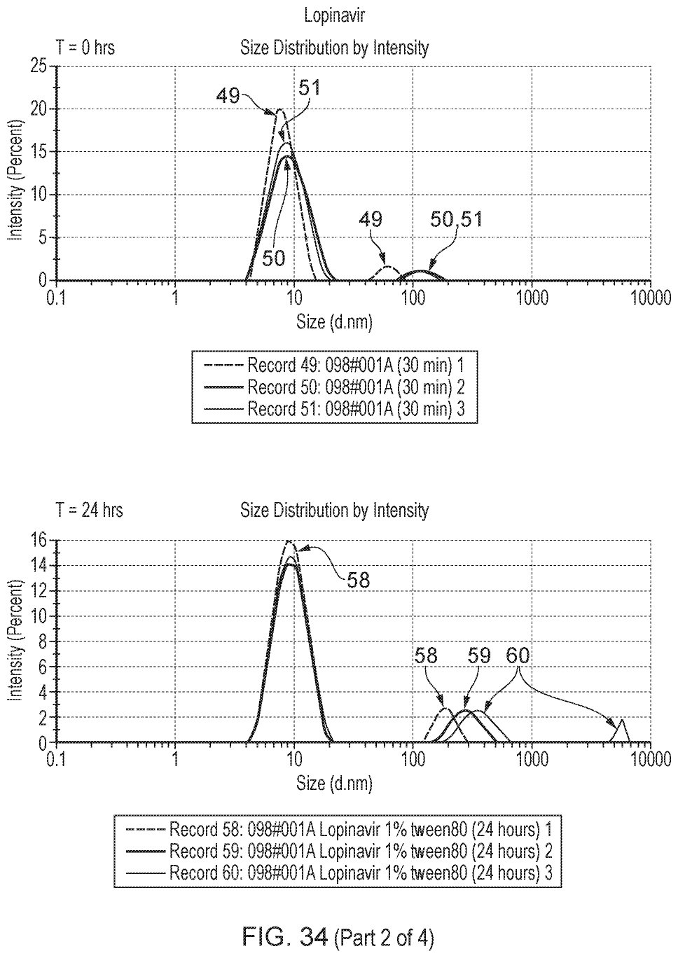

Residual moisture present in the formulations was quantified by loss on drying by DVS before and after storage (FIG. 31). A small decrease in residual moisture was observed.

Conclusions

A sample of the lead formulation was stored at 37° C. for 7 days. No significant difference was observed between this sample and the original material analysed at t=0 in terms of its reconstituted solution A600 performance, and the residual moisture in the powder. A slight increase in the trehalose glass transition temperature was observed after 7 days storage, in the DSC analysis.

Overall Conclusion of Examples 1-6

From an initial candidate formulation, feed solution preparation and spray drying methods were developed to prepare a complex of AP1903 and rHA which could be dissolved to give at least 0.3 mg/mL AP1903 in the finally prepared solution in D5W, which is suitable for intravenous (IV) infusion. Absorbance at 600 nm was used to measure turbidity, and hence solubility of the formulation, and absorbance was shown to be low and stable over 24 hours. AP1903 and rHA were found to form nanoparticles in solution, and these were measured. The results suggest that the AP1903 complex is not tightly bound, and that the nanoparticles formed may transient in nature, such that AP1903 and rHA are constantly fluxing in and out of complex. Stability of the lead candidate formulation was shown over 7 days stored at 37° C./ambient RH.

### Example 7—Development of Spray Dried Formulations of Poorly Soluble Apis with Recombinant Human Albumin

Introduction

Experimental studies were undertaken to develop a spray dried formulation of a poorly soluble API, recombinant human albumin (rHA) and trehalose. The dry powder formulation was produced by spray drying all of the ingredients from a single water/ethanol solution, or water/DMSO solution, to produce a stable spray dried dispersion (SDD). The dry powder formulations were shown to improve the solubility of the API in water, compared to that of the unformulated API, by formation of a nanoparticulate complex.

The rationale behind this study was to investigate whether the SDDs produced would have improved aqueous dispersion/solubility properties compared to the unformulated APIs, selected from BCS class II and IV compounds exhibiting poor solubility/bioavailability.

Examples 1-6 exemplified the development of a method for producing a spray dried dispersion (SSD) of Rimiducid, recombinant human albumin and trehalose. The dry powder formulation was shown to improve the solubility of the API in water, compared to that of the unformulated Rimiducid, by formation of a nanoparticulate complex. The present example describes a series of experimental studies to investigate the applicability of this method to other poorly soluble APIs.

Nine poorly soluble APIs were investigated; Acyclovir, Aripiprazole, Bifonazole, Etravirine, Ezetimibe, Glibenclamide, Lopinavir, Phenytoin and Ritonavir. Initial investigations reduced this down to five by assessing the APIs solubility in ethanol and ethanol water mixtures; Bifonazole, Ezetimibe, Lopinavir, Phenytoin and Ritonavir.

Five spray dried formulations were produced and reconstituted into a 5 wt % dextrose solution (D5W) at a nominal API concentration of 0.3 mg/mL. The Lopinavir formulation required addition of 1% Tween 80 (polysorbate 80) to successfully disperse within the D5W solution. Analysis showed a considerable increase in API solubility, for all five spray dried formulations, compared to the unformulated APIs.

Dimethyl sulfoxide was investigated as an alternative solvent to ethanol in the preparation of the spray dried dispersion of Ezetimibe. A spray dried formulation was produced; upon analysis the rHA was seen to have a significant increase in dimer compared to the same formulation produced using ethanol. However, no negative effects upon the performance of the formulation were apparent, with similar increases in API solubility observed.

Materials

TABLE 15
Materials used for batch production and analysis

| Item | Supplier | Lot/Batch No. |
|---|---|---|
| Acyclovir | Sigma Aldrich | LRAA9058 |
| Aripiprazole | — | 160501 |
| Bifonazole | Sigma Aldrich | LRAA9092 |
| Etravirine | Advanced ChemBlocks | 10441 |
| Ezetimibe | Sigma Aldrich | LRAB0503 |
| Glibenclamide | Sigma Aldrich | LRAA9084 |
| (Glyburide) | | |
| Lopinavir | Sigma Aldrich | 116M4711V |
| Phenytoin | Sigma Aldrich | LRAA8984 |
| Ritonavir | Sigma Aldrich | LRAA8003 |
| Recombumin ® Prime | Albumedix | 1907 |
| rHA | | |
| HPLC grade water | Fisher Scientific | 1735453 |
| Ethanol | Fisher Scientific | 1722802, 1727232 |
| Dimethyl sulfoxide | Fisher Scientific | 1729536 |
| (DMSO) | | |
| Dextrose | Fisher Scientific | SLBQ9668V |
| Polysorbate 80 | Croda | 1176143 |

Methods

Ethanol Solubility Testing

Solubility in Ethanol

The solubility of nine poorly soluble APIs in ethanol was investigated; Acyclovir, Aripiprazole, Bifonazole, Etravirine, Ezetimibe, Glibenclamide. Lopinavir, Phenytoin and Ritonavir. For each API, approximately 20 mg was weighed into a scintillation vial. 3 mL of ethanol was added to each vial. Samples were vortexed until completely dissolved. If after 2 mins the sample was not dissolved, a further 3 mL of ethanol was added, and the sample was vortexed again. After another 2 mins, if the sample was still not dissolved, the API in question was classed as insoluble. For Aripiprazole, extra ethanol was added (9 mL total) to give an approximate concentration of 2.2 mg/mL before it was completely solubilised.

Ethanol Water Mixtures

APIs were dissolved in ethanol at the desired concentration (Tables 16 and 17). The required volume of this solution was added to a 50:50 solution of ethanol:HPLC grade water with stirring (Table 17).

DMSO Solubility Testing

1 mL of DMSO was added to a scintillation vial containing approximately 20 mg of Ezetimibe. The sample readily dissolved with gentle mixing.

Feed Solution Preparation

Five APIs were selected for spray drying, based upon their solubility in ethanol, and ethanol/water. These are shown in Table 16.

Based upon Examples 1-8, the feed solutions were prepared with the quantities of all components calculated to maintain the following:
Total solids concentration in the feed solution of 3.2% w/v;
3:1 molar ratio of API:rHA;
1:1 weight ratio of rHA-trehalose;
40:60 volume ratio of EtOH:H₂O.

In calculating the molar ratio of each API to rHA, the molecular weight values shown in Table 16 were used.

TABLE 16
Molecular weight values

| | | Weight |
|---|---|---|
| | Molecular weight | 13.5 μmol API |
| API | g/mol | (mg) |
| Bifonazole | 310.392 | 4.19 |
| Ezelimibe | 409.4 | 5.53 |
| Lopinavir | 628.81 | 8.49 |
| Phenytoin | 252.268 | 3.41 |
| Ritonavir | 720.946 | 9.73 |
| rHA | 66438 | |

Each feed solution contained 300 mg rHA (4.5 μmoles). For 3:1 molar ratio of API to rHA, 13.5 μmoles of each API was added.

The feed solution preparation method is designed to keep the API in solution, and avoid denaturation of the rHA by the ethanol. For all feed solutions, the preparation method was in a series of six steps as follows, all weights and volumes are shown in Table 17:
I. A solution of the API in ethanol was prepared (Solution 1);
II. 1.5 mL of Recombumin rHA (20%, 300 mg) was diluted to the required volume (see Table 17) with HPLC grade water (Solution 2);
III. A 1:1 (v/v) mixture of ethanol and HPLC grade water was prepared;
IV. The API solution (solution 1) was added dropwise to the 1:1 v/v Ethanol/water (solution 3), with stirring (solution 4);
V. Solution 4, was then added dropwise to the rHA solution (solution 2) with stirring;
VI. Trehalose was added at a weight ratio of 1:1 with the rHA, and the solution mixed gently until it was fully dissolved.

TABLE 17
Feed solution components

| | | Solution 2 | Solution 3 | Total Solids | % API in |
|---|---|---|---|---|---|
| | Solution 1 | (rHA Solution | (1:1 v/v | (including | formulation |
| API | (API in ethanol) | aq) | Ethanol/water) | trehalose) | w/w |
| Bifonazole | 4.2 mg in 3.0 ml | 300 mg in 6.8 ml | 9.0 ml | 604.2 mg | 0.7 |
| Ezetimibe | 5.5 mg in 3.0 ml | 300 mg in 6.8 ml | 9.0 ml | 605.5 mg | 0.9 |
| Lopinavir* | 20.0 mg in 3.0 ml ethanol | 300 mg in 6.9 ml | 9.5 ml | 620 mg | 3.2 |
| Phenytoin | 3.4 mg in 3.0 ml | 300 mg in 6.8 ml | 9.0 ml | 603.4 mg | 0.6 |
| Ritonavir | 9.8 mg in 3.0 ml | 300 mg in 6.8 ml | 9.0 ml | 609.8 mg | 1.6 |

*Incorrectly prepared at 7:1 molar ratio

Spray Drying

All feed solutions were spray dried using a Buchi B-290 spray dryer fitted with a Schlick two-fluid nozzle. The spray dryer was fitted with a high-performance cyclone. Solutions were pumped into the spray dryer using a Masterflex peristaltic pump at approximately 3 g/min. The solution was atomised with compressed air at a pressure of 1 bar. An outlet temperature of 65° C. was used for all samples.

The spray dried powders were collected, and stored refrigerated in sealed glass vials.

Dissolution Testing

The spray dried powders were reconstituted by first resuspending in HPLC grade water at a solids concentration of 200 mg/mL (the spray dried ritonavir formulation was made up at 132 mg/mL, as additional water was required to fully wet the powder). Once homogenous, this solution was transferred into a 5% dextrose in water (D5W) solution using a needle and syringe to give the final nominal concentration of 0.3 mg/mL API. A 1 mL sample was taken and mixed with 4 mL of 0.1% trifluoroacetic acid (TFA) in acetonitrile. Once the solution had gone clear, 1 mL was removed and passed through an Agilent Captiva ND cartridge. The filtrate was then analysed by HPLC to determine the concentration of API present in solution.

Solubility of APIs In D5W Solution

A saturated solution of Bifonazole, Lopinavir, Ritonavir, Ezetimibe and Phenytoin in D5W was prepared by adding 1 mL of D5W solution to approximately 10 mg of API and vortexing for 2 minutes. The solution was sampled after centrifugation (13.5 k rpm for 5 minutes) and the solubility of the APIs was determined by HPLC analysis of the saturated solution.

API Loading

Loading measurements were undertaken based on a bioanalysis method for AP1903 supplied by Southern Research. The method was assumed to be applicable for the multiple APIs investigated in this study. Samples were acidified by the addition of TFA, which denatured the rHA. The rHA and API in question were then separated using solid phase extraction. In a typical extraction 20 mg of spray dried formulation was added to 1 mL of HPLC grade water and mixed to form a solution. 4 mL of 0.1% TFA in acetonitrile was then added to this solution and mixed. Once the solution had gone clear, 1 mL was taken and passed through an Agilent Captiva ND cartridge. The filtrate was then analysed by HPLC.

HPLC Analysis

Analysis was performed on an Agilent 1200 series HPLC employing a Waters, Xbridge Peptide BEH C18 column (150×4.6 mm i.d., 3.5 μm). Lopinavir, Ritonavir, Ezetimibe and Phenytoin were analysed using similar methods, utilising a 1 mL/min flow rate of 60:40 HPLC acetonitrile:10 mM Phosphate buffer (pH 3±0.1) with UV detection at 220, 220, 232 and 208 nm respectively. Bifonazole was analysed using a 1 mL/min flow rate of 65:35 HPLC methanol:ammonium acetate (5 g/L, pH 2) with UV detection at 252 nm.

Size Exclusion Chromatography

Samples were prepared by first dissolving the spray dried powder in DI water at concentration of 200 mg/mL. Once dissolved, this solution was transferred into D5W using a needle and syringe to give the final nominal concentration of 0.3 mg/mL API. Size exclusion chromatography was performed on an Agilent 1100 series HPLC employing a TsK gel G3000SWXL (300×7.8 mm I.d., 5 μm) column, an isocratic flow of phosphate buffer (pH 7±0.1) at 1 mL/min with UV detection at 210 nm.

UV/Vis Analysis at 600 nm (A600)

For A600 analysis, the samples were analysed in D5W solution, at the same concentration used in the size exclusion chromatography. Samples were analysed on a Thermo Scientific Varioskan lux multimode microplate reader. Samples were plated up in a 96 well Maxisorp Nunc-immuno plate, 200 μL in triplicate vs. a 200 μL D5W blank in triplicate, and UV detection was set to 600 nm on a kinetic loop every 30 minutes for 24 hours (pulsed shaking for 30 seconds every 4 minutes at 300 rpm).

Zetasizer® Analysis

For nanoparticle size analysis, samples were analysed in D5W solution at the concentration used in the A600 measurements, using a Malvern Instruments Zetasizer® Nano S. Samples were contained in a 2 mL polystyrene cuvette.

Results and Discussion

Solubility

Ethanol Solubility

To quickly assess the APIs applicability to this method, the solubility of each API in ethanol was tested. Any API with solubility less than 1.2 mg/mL was considered insoluble and ruled out from further work (Table 18). Six of the nine APIs were deemed soluble enough to continue on the next stage of testing.

TABLE 18
Solubility of APIs in ethanol (>1.2 mg/mL)

| API | Soluble (>1.2 mg/mL) in EtOH? |
|---|---|
| Acyclovir | No |
| Aripiprazole | Yes |
| Bifonazole | Yes |
| Etravirine | No |
| Ezetimibe | Yes |
| Glibenclamide | No |
| Lopinavir | Yes |
| Phenytoin | Yes |
| Ritonavir | Yes |

Ethanol Water Mixtures

To prepare the spray drying feed solution the API should preferably be soluble in 40:60 (v/v) ethanol/water mixtures. Any API that precipitated on addition of the ethanolic solution (from the previous section) to a 50:50 solution of EtOH:H₂O was eliminated from the study. Five of the remaining six APIs passed this stage of testing (Table 19).

TABLE 19
Solubility of APIs in ethanol water mixtures.

| | Soluble in EtOH/H₂O |
|---|---|
| API | mixture |
| Aripiprazole | No |
| Bifonazole | Yes |
| Ezetimibe | Yes |
| Lopinavir | Yes |
| Phenytoin | Yes |
| Ritonavir | Yes |

Spray Drying

Product Yields

The five feed solutions were successfully spray dried, producing white free flowing powders, with high yields. Product yields obtained for each formulation are detailed in Table 20.

TABLE 20
Yields of spray dried formulation for each API.

| Spray dried API | |
|---|---|
| formulation | Yield |
| Bifonazole | 77% |
| Ezetimibe | 84% |
| Lopinavir | 75% |
| Phenytoin | 76% |
| Ritonavir | 60% |

API Loading

The percentage API present in the spray dried formulations was determined using the method described above (Example 7, Methods, API Loading). The loadings and their percent assay of the expected amount—based on the nominal—are shown in Table 21.

This method was optimised for the quantification of Rimiducid. and its accuracy has not been tested for these APIs. For all but one formulation (Ritonavir) the percent loading is within 120% of the nominal amount. The ritonavir formulation showed a loading of 2.18% vs. the expected value of 1.6%.

TABLE 21
Percentage of API contained within the
SD formulations vs. the nominal.

| SD API/rHA | API Loading | % Assay |
|---|---|---|
| Bifonazole | 0.66% | 95% |
| Ezetimibe | 0.80% | 88% |
| Lopinavir | 3.65% | 115% |
| Phenytoin | 0.60% | 105% |
| Ritonavir | 2.18% | 138% |

Dissolution Testing

Solubility of APIs in D5W Solution

The solubility of Bifonazole, Ezetimibe, Lopinavir, Phenytoin and Ritonavir in D5W solution were determined, as described above (Example 7, Methods, Solubility of APIs in D5W solution). The results are shown in Table 22, alongside the literature values of the APIs solubility in water.

TABLE 22
Solubility of APIs in water and D5W solution.

| | Solubility in water | Solubility in D5W solution |
|---|---|---|
| API | (literature) (mg mL⁻¹) | (mg mL⁻¹) |
| Bifonazole | 0.0025 | 0.0035 |
| Ezetimibe | 0.0085 | 0.0034 |
| Lopinavir | 0.0019 | 0.0149 |
| Phenytoin | 0.0711 | 0.0141 |
| Ritonavir | 0.0013 | 0.0026 |

Reconstitution of Spray Dried Powders in D5W Solution

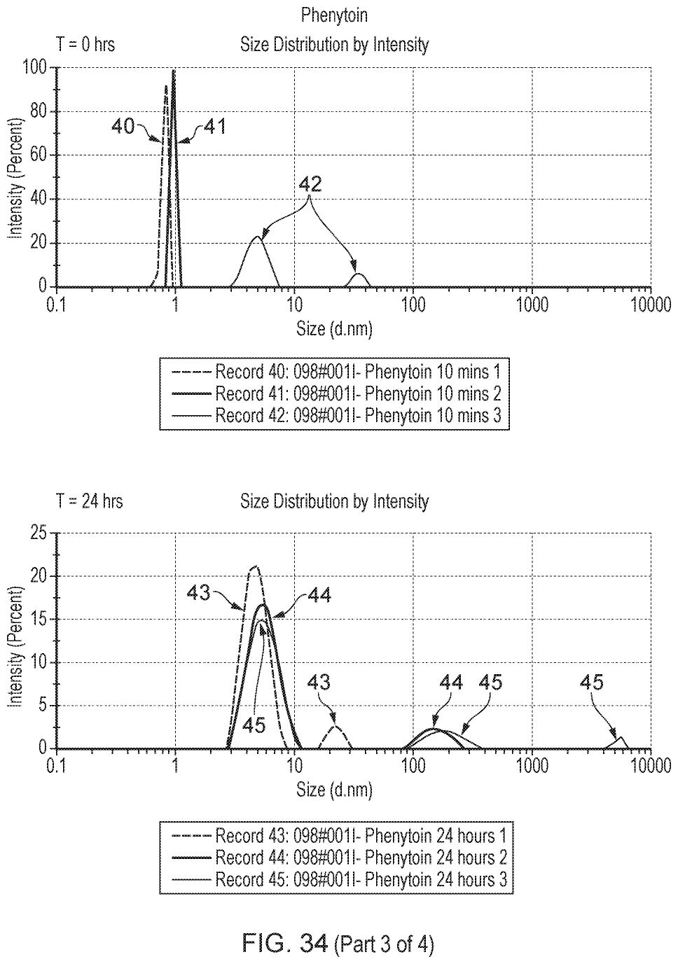

The spray dried formulations were reconstituted in D5W to a nominal concentration of 0.3 mg/mL API. All but one formulation (Lopinavir) reconstituted easily into D5W (FIG. 32). It was difficult to achieve wetting of the Lopinavir formulation, with a large number of particles seen floating on the surface. To improve the wetting of the particles a surfactant was added to the D5W solution, 1% (v/v) Tween 80 (polysorbate 80). The presence of the surfactant resulted in a homogeneous suspension, which was analysed along with the other reconstituted formulations by HPLC, A600 measurements, Zetasizer® and SEC. On reinvestigation, it was found that this formulation was mistakenly prepared at an API:rHA molar ratio of 7:1 rather than the intended 3:1, which may explain this difficulty with reconstitution.

Enhanced API Solubility Measurements

The concentration of each API present in the D5W solution was determined using the API loading HPLC method described above (Example 7, Methods, API Loading). The nominal concentration for all formulations was 0.3 mg/mL API. For all but one of the SD formulations the calculated concentrations were lower than the expected 0.3 mg/mL (Table 23). The phenytoin formulation had a higher than nominal concentration, but this is to be expected as the formulation has a slightly higher API loading (138% of nominal). Table 23 also shows the percentage increase in solubility of the formulated API compared to the unformulated API in D5W solution. All formulations show in excess of a 1000-fold increase in solubility compared to the unformulated API. This is a significant increase in API solubility, demonstrating the effectiveness of this method in improving the aqueous solubility of poorly soluble APIs. The SD formulation of Lopinavir was analysed twice, once for the dissolution that failed to fully wet, and once for the formulation dispersed in D5W/1% Tween 80 (polysorbate 80). The initial solution shows an increase of 629% compared to the unformulated API. However, when the dissolution medium contains Tween 80 (polysorbate 80), this increases to 1267%. The reconstituted solutions were further analysed by A600, Zetasizer® and SEC.

TABLE 23
Concentration of APIs in the D5W dissolution and the
percentage increase over the solubility of unformulated
API in D5W. ( ) = Reconstituted in D5W 1% Tween 80.

| | API Concentration in D5W | |
|---|---|---|
| SD API/rHA | solution (mg/mL) | % Increase in solubility* |
| Bifonazole | 0.188 | 5327% |
| Ezetimibe | 0.202 | 5919% |
| Lopinavir | 0.094 (0.189) | 629% (1267%) |
| Phenytoin | 0.342 | 2422% |
| Ritonavir | 0.226 | 8819% |

*Compared with unformulated API (see Table 22)

A600 Measurements

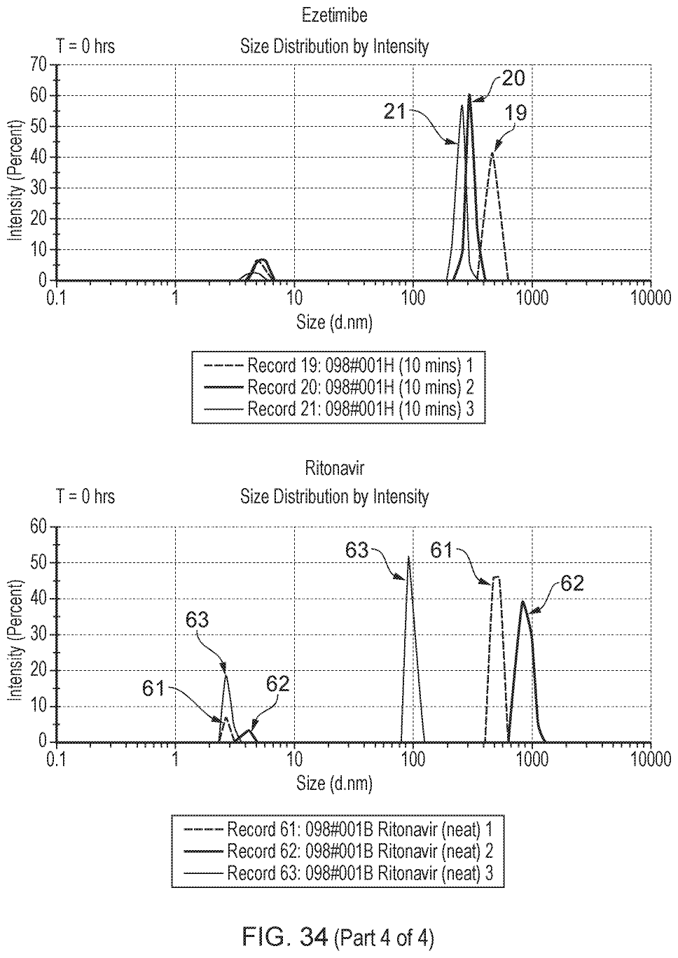

The formulations reconstituted in D5W were analysed by UV-vis analysis at a wavelength of 600 nm to give a measure of the turbidity of the solutions over 24 hours (FIG. 33). Triplicate repeat measurements show reproducibility for all formulations, with the exception of the Ritonavir formulation which seems to deviate from the mean by a significant amount, towards the end of the analysis. Although all formulations seem to follow a similar trend of slowly increasing in A600 over the course of 24 hours, the extent of this increase seems to be individual to each formulation. The Bifonazole formulation shows the largest increase in absorbance over the course of 24 hours with the Lopinavir formulation (in D5W 1% Tween 80 (polysorbate 80)) showing almost no increase in absorbance over 24 hours. The presence of surfactant in the reconstituted Lopinavir formulation could explain the inherently low turbidity, with the Tween stabilising the nanoparticles in the nanosuspension.

Zetasizer® Analysis

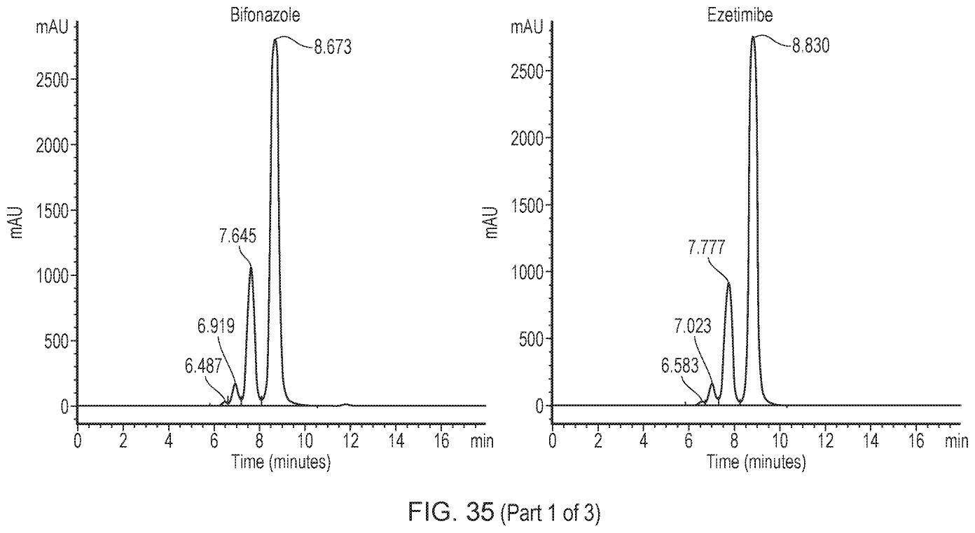

FIG. 34 shows the Zetasizer® traces for the reconstituted SD powders at T=0 and 24 hours. The Lopinavir and Phenytoin suspensions appeared to be largely stable over 24 hours. The particle size distributions for both the Bifonazole and Phenytoin suspensions showed that over the entire 24 hours period, some larger particles had formed, which may have also been indicated by A600 results as an initial increase in turbidity followed by a plateau. The Lopinavir suspension shows stable and small nanoparticles over 24 hours correlating well with the small increase in A600 over the same timescale. The stability of the particle size distributions over 24 hours was not established for the Ritonavir and Ezetimibe suspensions as the intercept from the Zetasizer® exceeded one, reducing the validity of the data obtained. Overall there is good agreement between the A600 and Zetasizer® data. Formulations that show a greater increase in turbidity also show the growth of larger particles over the 24 hours, while those with a small and almost negligible change in turbidity show a stable particle distribution over 24 hours.

SEC-HPLC

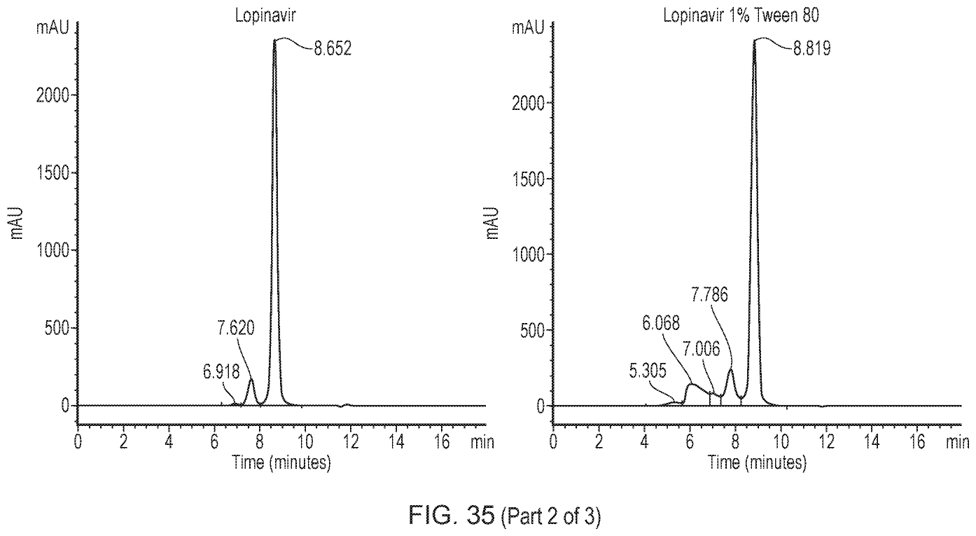

The reconstituted formulations were analysed by SEC-HPLC to investigate the integrity of the rHA. The presence of larger molecular weight structures could indicate degradation/aggregation of the albumin possibly leading to insoluble components of the formulations. FIG. 35 shows the resultant chromatograms and Table 24 details the relative percentage of the different peaks, defined as rHA monomer, dimer, trimer, tetramer and polymer (Retention time (Rt) of approximately 8.6, 7.6, 6.9, 6.4 and 5.3 minutes respectively). Two traces for Lopinavir are shown, one corresponding to the initial attempt to reconstitute the SD formulation and the other to the successful attempt containing 1% Tween 80 (polysorbate 80) in the D5W solution. The difference between the two is quite striking, the presence of the surfactant has solubilised the higher molecular weight rHA structures. This is interesting as the API remains soluble despite their presence, so much so that this formulation has the lowest increase in turbidity over 24 hours (FIG. 33).

The Bifonazole and the Ezetimibe formulations show the largest increase in turbidity over 24 hours and show a larger percentage of rHA dimer in the formulation, 23% vs. 8% for the formulations of Phenytoin and Lopinavir. It seems the stability of the formulations might be linked to the presence of the higher molecular weight structures, excluding the Lopinavir formulation reconstituted in D5W with 1% Tween 80 (polysorbate 80), in which the addition of the surfactant has solubilised these structures.

TABLE 24
The relative peak area percentages obtained from
SEC, defined as rHA monomer, dimer, trimer, tetramer
and polymer (Rt of approximately 8.6, 7.6, 6.9,
6.4 and 5.3 minutes respectively).

| | Monomer | Dimer | Trimer | Tetramer | Polymer |
|---|---|---|---|---|---|
| SD API/rHA | (%) | (%) | (%) | (%) | (%) |
| Bifonazole | 73 | 23 | 3 | 0.5 | — |
| Ezetimibe | 73 | 23 | 3 | 0.5 | — |
| Lopinavir | 91 | 8 | 1 | — | — |
| Lopinavir (1% | 71 | 11 | 3 | 13 | 2 |
| Tween 80) | | | | | |
| Phenytoin | 91 | 8 | 1 | — | — |
| Ritonavir | 88 | 11 | 1 | — | — |

DMSO as an Alternative Solvent to Ethanol

Solubility in DMSO

A solution preparation method that utilised an alternative solvent was investigated. The alternative solvent needed to be similar to ethanol in its ability to dissolve the APIs whilst being miscible with water. Dimethyl sulfoxide (DMSO) was chosen as a promising candidate. Ezetimibe was chosen as a suitable API owing to its favourable COSHH assessment combined with the knowledge of DMSO as a transport agent. The solubility of Ezetimibe in DSMO was assessed by dissolving approx. 20 mg of API in 1 mL of solvent. The API dissolved readily with gentle agitation. The next step was to assess the API solubility in a mixture of DMSO and water. The API remained in solution upon addition to a 50:50 DMSO:water mixture.

Feed Solution Preparation

The feed solution was prepared as described above (Example 7, Methods, Feed Solution Preparation). All quantities used were as shown for this API in Table 17, with the replacement of ethanol by DMSO. The feed solution produced was clear and suitable for spray drying with no signs of precipitation or degradation of the rHA.

Spray Drying

The prepared feed solution was spray dried as above. The product was a white free flowing powder with little deposition on the cyclone walls and a yield of 78%.

API Loading

The percentage of Ezetimibe present in the spray dried formulation was calculated using the method described above (Example 7, Methods, API Loading). The loading was 74% of the expected, at 0.71% Ezetimibe in the formulation (Table 25). The Ezetimibe formulation processed with DMSO produced similar values to the Ezetimibe formulation processed with ethanol.

TABLE 25
Percent API loading

| SD API/rHA | API Loading | % Assay |
|---|---|---|
| Ezetimibe | 0.71% | 74% |

Reconstitution of the Spray Dried Powder in D5W Solution

The spray dried formulation was reconstituted without issue in D5W to a nominal concentration of 0.3 mg/mL API. The concentration of Ezetimibe present in the D5W solution was calculated using the API loading method, with the initial 1 mL sampled directly from the dissolution. A concentration of 0.160 mg/mL Ezetimibe was calculated, resulting in an increase of 4673% over the solubility of unformulated Ezetimibe in D5W solution (Table 26). These values were similar to the Ezetimibe formulation processed with ethanol.

TABLE 26
Concentration of Ezetimibe in dissolution of reconstituted spray-dried
(SD) powder and its increase in solubility over unformulated API.

| | Concentration in D5W | Increase over D5W |
|---|---|---|
| SD API/rHA | solution (mg/mL) | solubility |
| Ezetimibe | 0.160 | 4673% |

A600 Measurements

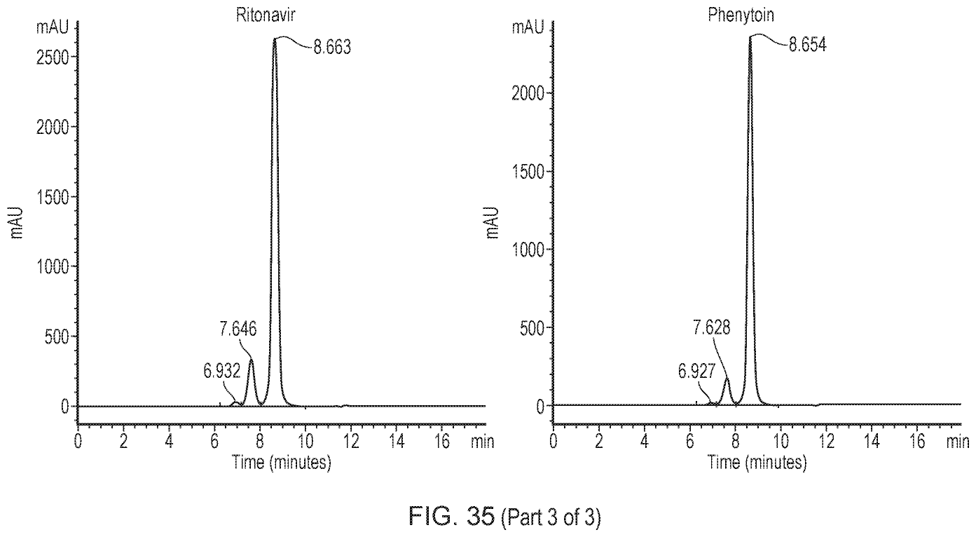

A sample of the dissolution was analysed by UV-vis at 600 nm over 24 hours (FIG. 36). The absorbance increases slowly over time, as was observed with the other formulations. However, the Ezetimibe formulation processed with DMSO increased less in 24 hours than the Ezetimibe formulation processed with ethanol did in the first hour (FIG. 33 and FIG. 36).

Zetasizer® Analysis

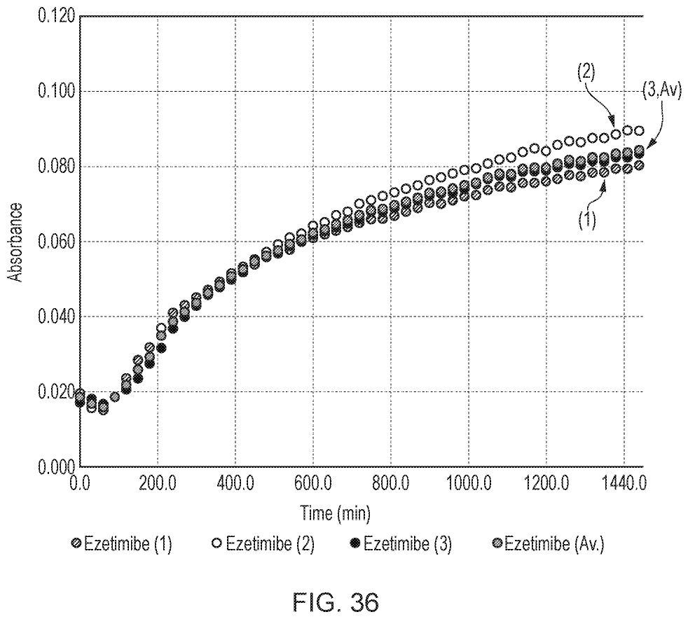

FIG. 37 shows the Zetasizer® analysis of the dissolution in D5W. As seen previously, formulations with lower turbidity over 24 hours tend to show greater stability in the Zetasizer® analysis. A similar case is seen here, with similar particle size distributions present at T=0 and T=24 hours.

SEC-HPLC

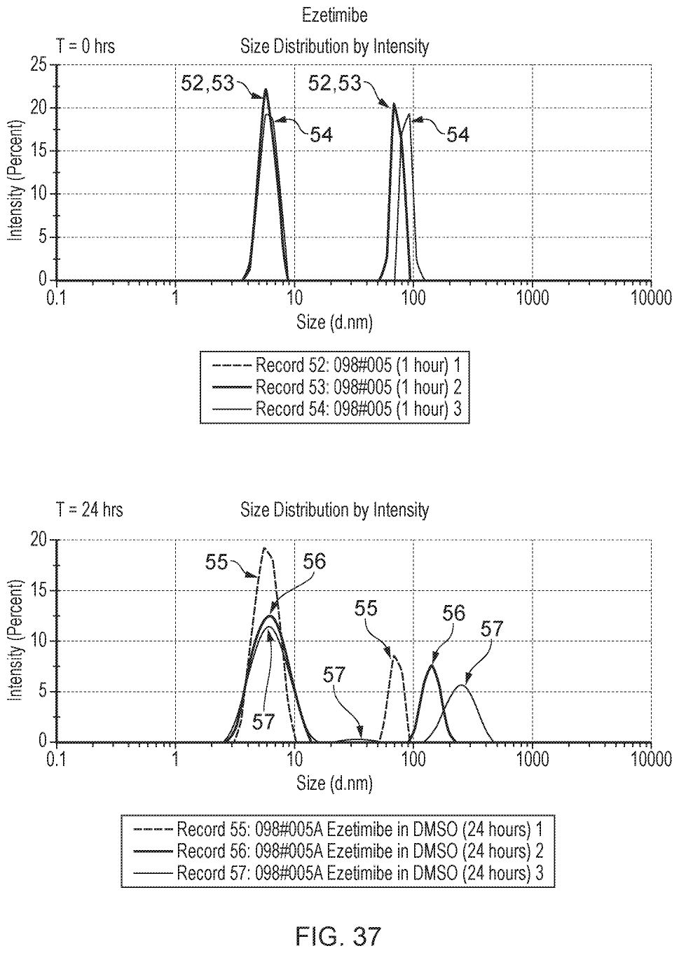

The reconstituted formulation was analysed by SEC-HPLC to investigate the effect of the API and DMSO on the albumin. It was a concern that, unlike the ethanol previously, the feed solution preparation process had not been optimised for DMSO and the albumin may be damaged by the high DMSO concentrations. FIG. 38 and Table 27 show a high percentage of rHA dimer in the formulation. It appears that the presence of DMSO in the formulation preparation has caused the growth of these higher molecular weight rHA structures. However, this does not seem to have negatively impacted the A600 or Zetasizer® analysis. In fact, this formulation has a lower and more stable turbidity than the Ezetimibe formulation produced when using ethanol. It is possible that the rHA dimer has a higher affinity towards binding the API than the monomer does creating the more stable dissolution seen in the A600 and Zetasizer® analysis.

TABLE 27
The relative peak area percentages obtained from SEC, defined
as rHA monomer, dimer, trimer, tetramer and polymer (Rt of
approximately 8.6, 7.6, 6.9, 6.4 and 5.3 mins respectively).

| | % | % | % | % | % |
|---|---|---|---|---|---|
| SD API/rHA | Monomer | Dimer | Trimer | Tetramer | Polymer |
| Ezetimibe | 46 | 43 | 9 | 2 | — |

Conclusion

Out of the initial nine APIs chosen for this study, four APIs, Acyclovir, Aripiprazole, Etravirine and Glibenclamide were excluded from this initial study using ethanol as a solvent due to poor solubility in either ethanol or an ethanol water mixture. The final five remaining APIs, Bifonazole, Ezetimibe, Lopinavir, Phenytoin and Ritonavir were spray dried with recombinant human albumin and trehalose, as in Examples 1-6 using Rimiducid. The formulations were analysed and all showed a considerable enhancement of API aqueous solubility, through formation of a stable nanosuspension, further supporting this method as a platform for improving poorly soluble APIs. The use of an alternative solvent DMSO, was shown to have similar results, improving the solubility of formulated Ezetimibe compared to the starting API. The stability of the albumin in the presence of DMSO was raised as a possible drawback to this solvent since the percentage of rHA dimer in the formulation increased. However, no other detriment to the formulation was observed.

### Example 8—Research Studies Looking at the Effect of Trehalose on the Storage Stability of Spray Dried Recombinant Human Albumin

Study Outline

In total 2 studies were undertaken to evaluate the effect of excipient sugars, formulating and spray drying rHA with different levels of trehalose and sucrose.

In stability study 1 (Example 8A), we spray dried AlbIX® recombinant human albumin (Albumedix Limited, Nottingham, UK) with 0-20% w/w trehalose and sucrose compared to rHA alone. The spray dried powders were tested for their stability at 40° C. with monomer levels measured by HPLC size exclusion (HPLC-SEC).

In stability study 2 (Example 8B) the rHA used was Recombumin® Alpha (Albumedix Limited, Nottingham, UK), supplied in vials containing 50 ml of sterile 20% (w/v) solution. This study used a higher amount of trehalose (1:1 rHA:Trehalose) and compared spray drying with freeze drying. Spray drying was undertaken using a Buchi B-290 spray dryer and solution was spray dried under standard conditions at an outlet (drying) temperature of 55° C. Again, samples were tested for their stability at 40° C. with monomer levels measured by HPLC-SEC.

Results

Results of Stability Study 1 (Example 8A)

Table 28 shows a summary of monomer levels over 4 weeks' incubation of spray dried powders. Monomer level/stability improves with increasing trehalose compared to the unformulated spray dried powder and sucrose appeared to work as well as trehalose.

TABLE 28
HPLC-SEC analysis of rHA monomer levels in spray-dried powders over 4
weeks' incubation at 40° C.
Incubation Time (weeks)

| | Stabiliser | | | | | |
|---|---|---|---|---|---|---|
| Sample | Conc (w/w) | 0 | 1 | 2 | 3 | 4 |
| Liquid | N/A | 96.5 | N/A | | | |
| Spray dried: no | 0 | 96.3 | 86.5 | 94.8 | 96.1 | 95.4 |
| excipients | | | | | | |
| Trehalose | 2 | 96.2 | 87.4 | 85.6 | 87.7 | 87.1 |
| | 5 | 96.2 | 87.4 | 86.4 | 88.3 | 881 |
| | 10 | 96.4 | 87.4 | 86.4 | 88.3 | 88.1 |
| | 20 | 96.6 | 91.1 | 89.4 | 90.4 | 90.5 |
| Sucrose | 2 | 96.4 | 88.1 | 85.1 | 86.7 | 88.1 |
| | 5 | 96.6 | 88.0 | 86.5 | 87.5 | 90.1 |
| | 10 | 96.7 | 89.8 | 88.5 | 89.7 | 90.1 |
| | 20 | 96.8 | 89.3 | 88.3 | 89.9 | 90.4 |

Results of Stability Study 2 (Higher Trehalose Levels) (Example 8B)

Results at the higher trehalose level (1:1) confirmed even better retention of monomer structure than observed at the lower levels used in Example 8A.

On storage at 40° C. (for up to 28 days) there was no observable change in the either the spray dried or the freeze-dried formulations (Table 29). Both samples remained stable, with no evidence of polymer formation throughout the study. Side by side comparison of the HPLC-SEC profiles over the 28-day stability study confirmed no detectable polymer formation and no concomitant loss of monomer peak height/area. The individual monomer peaks were then collated (data not shown).

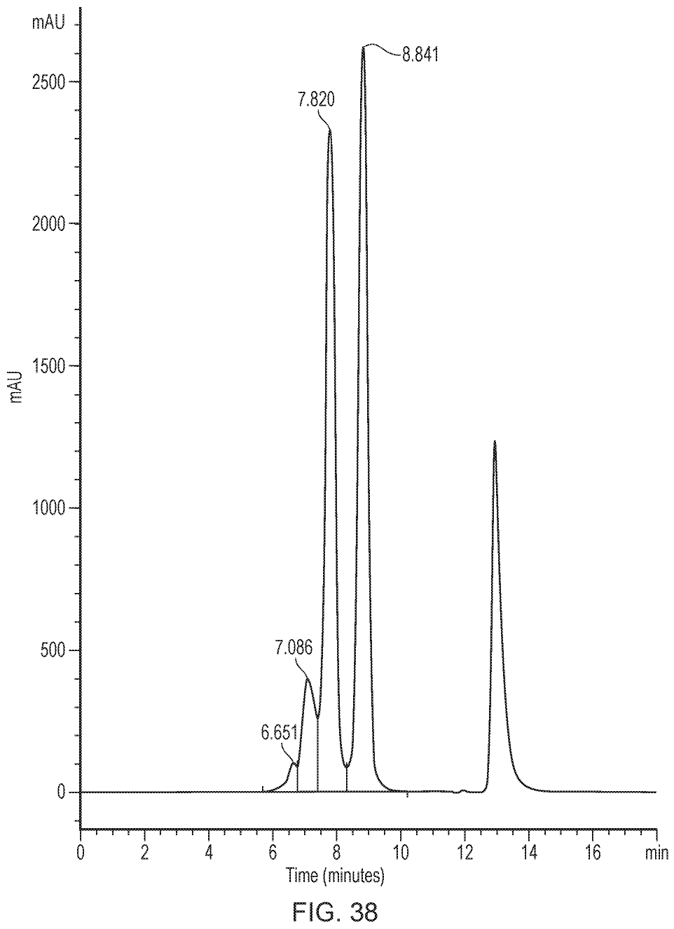

To assess whether any change in peak height or shape had occurred, peaks for all 5 times points (for each dried sample) were overlaid on a single elution profile, and as can be seen in FIG. 39, none of the peaks showed any change with time.

TABLE 29
HPLC-SEC analysis of spray dried and freeze
dried rHA after storage at 40° C.

| Sample | T = 0 | 7 d | 14 d | 21 d | 28 d |
|---|---|---|---|---|---|
| Spray dried rHA | 99.3 | 99.1 | 99.1 | 99.2 | 99.3 |
| (% monomer) | | | | | |
| Freeze dried rHA | 99.6 | 99.3 | 99.1 | 99.5 | 99.4 |
| (% monomer) | | | | | |

### Example 9—Initial Studies with Curcumin

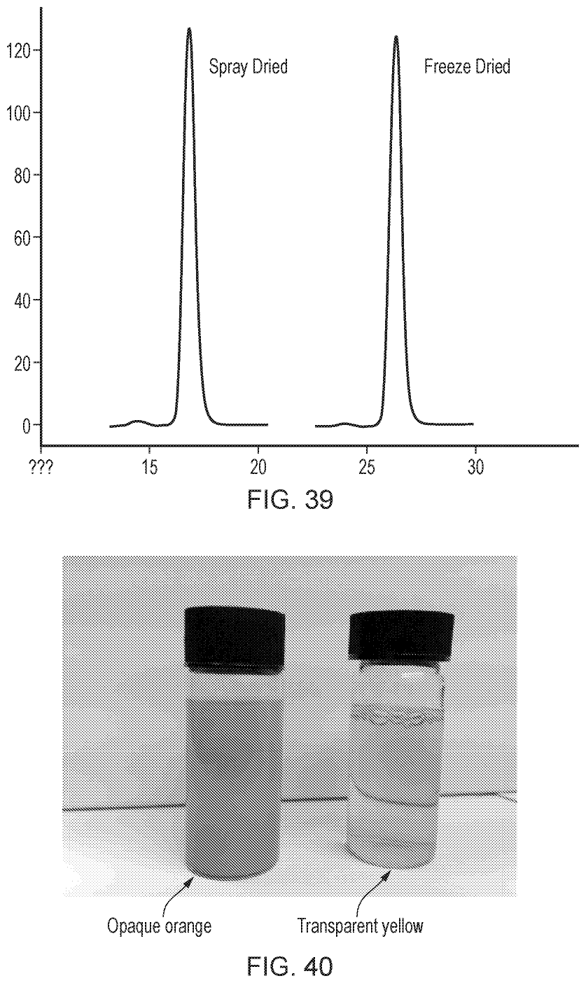

The potential for rHA to enhance the formulation/solubility of poorly soluble drugs was first noticed using curcumin as a model drug compound. Curcumin is poorly water soluble; however, when it was spray dried with rHA the subsequent spray dried dispersion produced showed excellent solubility (see FIG. 40). It is likely that curcumin is binding tightly to the rHA.

### Embodiments of the Invention

1. A method of enhancing the solubility and/or the rate of dissolution of a Class II or Class IV low solubility molecule, the method comprising spray-drying a mixture comprising the Class II or Class IV low solubility molecule, a water-miscible solvent, albumin and an agent that prevents self-aggregation of albumin.

2. The method according to Embodiment 1, wherein the method comprises (a) dissolving the Class II or Class IV low solubility molecule in a water-miscible solvent to form a solution, (b) mixing the solution of the Class II or Class IV low solubility molecule and water-miscible solvent with albumin and an agent that prevents self-aggregation of albumin, and (c) spray-drying the mixture.

3. The method according to Embodiment 1 or 2, wherein the low solubility molecule is a Class II molecule, or wherein the low solubility molecule is a Class IV molecule.

4. The method according to any one of Embodiments 1-3, wherein the low solubility molecule has a solubility in water of less than or equal to 10 mg/mL, 8 mg/mL, 6 mg/mL, 5 mg/mL, 4 mg/mL, 2 mg/mL, 1.5 mg/mL, 1 mg/mL, 0.8 mg/mL, 0.6 mg/mL, 0.4 mg/mL, 0.2 mg/mL, 0.1 mg/mL, 0.05 mg/mL, 0.02 mg/mL, 0.01 mg/mL, 0.005 mg/mL, 0.001 mg/mL, 0.0005 mg/mL or 0.0001 mg/mL.

5. The method according to any one of Embodiments 1-4, wherein the low solubility molecule has a solubility in water of greater than or equal to 0.00001 mg/mL, 0.0001 mg/mL, 0.0005 mg/mL, 0.001 mg/mL, 0.005 mg/mL, 0.01 mg/mL, 0.02 mg/mL, 0.05 mg/mL, 0.1 mg/mL, 0.2 mg/mL, 0.4 mg/mL, 0.6 mg/mL, 0.8 mg/mL, 1 mg/mL, 1.5 mg/mL, 2 mg/mL, 4 mg/mL, 5 mg/mL, 6 mg/mL or 8 mg/mL.

6. The method according to any one of Embodiments 1-5, wherein the low solubility molecule has a solubility in phosphate buffer of less than or equal to 10 mg/mL.

7. The method according to any one of Embodiments 1-6, wherein the low solubility molecule has a physiological charge of +1, 0 or −1.

8. The method according to any one of Embodiments 1-7, wherein the low solubility molecule has a number of rings greater than or equal to 1.

9. The method according to any one of Embodiments 1-8, wherein the low solubility molecule has a number of heterocyclic rings containing 3 to 7 molecules greater than or equal to 1.

10. The method according to any one of Embodiments 1-9, wherein the low solubility molecule has a molecular weight of less than or equal to 3000 g/mol.

11. The method according to any one of Embodiments 1-10, wherein the low solubility molecule has a molecular weight of greater than or equal to 150 g/mol.

12. The method according to any one of Embodiments 1-11, wherein the low solubility molecule is soluble in the water-miscible solvent, optionally wherein the low solubility molecule has a solubility in the water-miscible solvent of at least 1 mg/mL, 1.2 mg/mL, 1.4 mg/mL, 1.6 mg/mL, 1.8 mg/mL, 2.0 mg/mL, 2.2 mg/mL, 2.4 mg/mL, 2.6 mg/mL, 2.8 mg/mL or 3.0 mg/mL.

13. The method according to Embodiment 12, wherein the low solubility molecule is soluble in the water-miscible solvent comprising one or more water-miscible solvents selected from ethanol, acetic acid, acetone, dimethylsulphoxide, formic acid, 1-propanol, 2-propanol, tetrahydrofuran and N,N-dimethylformamide.

14. The method according to Embodiment 13, wherein the low solubility molecule is soluble in ethanol.

15. The method according to Embodiment 14, wherein the low solubility molecule has a solubility in ethanol of at least 1 mg/mL.

16. The method according to any one of Embodiments 1-15, wherein the low solubility molecule is a peptide, a small molecule, a nucleic acid, a carbohydrate, or a natural product.

17. The method according to any one of Embodiments 1-16, wherein the low solubility molecule has a solubility in water of less than or equal to 0.02 mg/ml, a physiological charge of between +1 and −1, and greater than or equal to 4 rings.

18. The method according to any one of Embodiments 1-17, wherein the low solubility molecule is selected from the compounds in Table A, optionally wherein the low solubility molecule is a Class II compound selected from the group: aceclofenac, albendazole, atovaquone, bicalutamide, clozapine, danazol, ezetimibe, fenofibrate, glibenclamide, itraconazole, lopinavir, modafinil, nabilone, nimesulide, nimodipine, paliperidone, phenytoin, propofol, prostaglandin E1, rapamycin, repaglinide, risperidone, ritonavir, tacrolimus, teniposide, tretinoin, valsartan, vincristine, voriconazole, zipradisone; or a Class IV compound selected from the group: acyclovir, allopurinol, amoxicillin, amphotericin b, aripiprazole, bifonazole, carfilzomib, cefuroxime axetil, docetaxel, etravirine, linezolid, oxcarbazepine, paclitaxel, rimiducid.

19. The method according to Embodiment 18, wherein the low solubility molecule is a Class II compound selected from the group: danazol, ezetimibe, lopinavir, phenytoin, rapamycin, ritonavir and tacrolimus; or a Class IV compound selected from the group: bifonazole, etravirine and rimiducid.

20. The method according to any one of Embodiments 1-19, wherein the low solubility molecule is a Class IV molecule having a solubility in water less than 0.005 mg/ml, a physiological charge of 0, a number of rings greater than or equal to 4, and a molecular weight of less than 350 g/mol.

21. The method according to Embodiment 20, wherein the low solubility molecule is bifonazole.

22. The method according to any one of Embodiments 1-19, wherein the low solubility molecule is a Class II molecule having a solubility in water less than 0.05 mg/ml, a physiological charge of 0, a number of rings greater than or equal to 5, and a molecular weight of less than 350 g/mol.

23. The method according to Embodiment 22, wherein the low solubility molecule is danazol.

24. The method according to any one of Embodiments 1-19, wherein the low solubility molecule is a Class IV molecule having a solubility in water less than 0.05 mg/ml, a physiological charge of 0, a number of rings greater than or equal to 3, and a molecular weight of less than 450 g/mol.

25. The method according to Embodiment 24, wherein the low solubility molecule is etravirine.

26. The method according to any one of Embodiments 1-19, wherein the low solubility molecule is a Class II molecule having a solubility in water less than 0.01 mg/ml, a physiological charge of 0, a number of rings greater than or equal to 2, and a molecular weight of less than 450 g/mol.

27. The method according to Embodiment 26, wherein the low solubility molecule is ezetimibe.

28. The method according to any one of Embodiments 1-19, wherein the low solubility molecule is a Class II molecule having a solubility in water less than 0.005 mg/ml, a physiological charge of 0, a number of rings greater than or equal to 4, and a molecular weight of less than 650 g/mol.

29. The method according to Embodiment 28, wherein the low solubility molecule is lopinavir.

30. The method according to any one of Embodiments 1-19, wherein the low solubility molecule is a Class II molecule having a solubility in water less than 0.1 mg/ml, a physiological charge of 0, a number of rings greater than or equal to 3, and a molecular weight of less than 300 g/mol.

31. The method according to Embodiment 30, wherein the low solubility molecule is phenytoin.

32. The method according to any one of Embodiments 1-19, wherein the low solubility molecule is a Class IV molecule having a solubility in water less than 0.000002 mg/ml, a physiological charge of 0, a number of rings greater than or equal to 8, and a molecular weight of less than 1500 g/mol.

32a. The method according to any one of Embodiments 1-19, wherein the low solubility molecule is a Class II molecule having a solubility in water less than 0.002 mg/mL, a physiological charge of 0, a number of rings greater than or equal to 4, and a molecular weight of less than 950 g/mol.

33. The method according to Embodiment 32, wherein the low solubility molecule is rimiducid.

33a. The method according to Embodiment 32a, wherein the low solubility molecule is rapamycin.

34. The method according to any one of Embodiments 1-19, wherein the low solubility molecule is a Class II molecule having a solubility in water less than 0.005 mg/ml, a physiological charge of 0, a number of rings greater than or equal to 4, and a molecular weight of less than 750 g/mol.

35. The method according to Embodiment 34, wherein the low solubility molecule is ritonavir.

36. The method according to any one of Embodiments 1-19, wherein the low solubility molecule is a Class II molecule having a solubility in water less than 0.005 mg/ml, a physiological charge of 0, a number of rings greater than or equal to 4, and a molecular weight of less than 850 g/mol.

37. The method according to Embodiment 36, wherein the low solubility molecule is tacrolimus.

38. The method according to any one of Embodiments 1-19, wherein the low solubility molecule is a Class IV molecule having a solubility in water less than 0.01 mg/ml, a physiological charge of 0, a number of rings greater than or equal to 7, and a molecular weight of less than 900 g/mol.

39. The method according to Embodiment 38, wherein the low solubility molecule is paclitaxel.

40. The method according to any one of Embodiments 1-39, wherein the ratio of low solubility molecule to albumin is greater than approximately 1:50, or less than 5:1, or between 1:50 and 5:1.

41. The method according to any one of Embodiments 1-40, wherein the water-miscible solvent comprises one or more water-miscible solvents selected from ethanol, acetic acid, acetone, dimethylsulphoxide, formic acid, 1-propanol, 2-propanol, and tetrahydrofuran and N,N-dimethylformamide.

42. The method according to Embodiment 41, wherein the water-miscible solvent(s) comprises ethanol.

43. The method according to Embodiment 40 or 41, wherein the water-miscible solvents comprise ethanol and water.

44. The method according to any one of Embodiments 1-43, wherein prior to the method the albumin is in solution in a solvent comprising water.

45. The method according to Embodiment 44, wherein prior to the method the albumin solution comprises water and one or more water-miscible solvents.

46. The method according to Embodiment 45, wherein prior to the method the albumin solution comprises water and ethanol.

47. The method according to any one of Embodiments 1-46, wherein the result prior to spray-drying is a single-phase solution of the low solubility molecule, water-miscible solvent(s), albumin and the agent that prevents self-aggregation of albumin.

48. The method according to any one of Embodiments 1-47, wherein the albumin is a recombinant albumin.

49. The method according to any one of Embodiments 1-48, wherein the albumin is human albumin.

50. The method according to Embodiment 48 or 49, wherein the albumin is recombinant human albumin.

51. The method according to Embodiment 50, wherein the recombinant human albumin is a fusion, variant or derivative.

52. The method according to any one of Embodiments 1-51, wherein the agent that prevents self-aggregation of albumin is a sugar, modified sugar or sugar derivative.

53. The method according to Embodiment 52, wherein the sugar, modified sugar or sugar derivative is a non-reducing sugar.

54. The method according to Embodiment 52 or 53, wherein the sugar, modified sugar or sugar derivative has a high glass transition point.

55. The method according to any one of Embodiments 52-54, wherein the sugar, modified sugar or sugar derivative is selected from one or more of trehalose, sucrose and dextrose.

56. The method according to Embodiment 55, wherein the sugar, modified sugar or sugar derivative is trehalose.

57. The method according to any one of Embodiments 1-51, wherein the agent that prevents self-aggregation of albumin is a polymer.

58. The method according to Embodiment 57, wherein the polymer is a synthetic polymer, a natural polymer, a sugar polymer or a polysaccharide, preferably wherein the polymer is suitable for parenteral delivery, e.g. intravenous administration.

59. The method according to any one of Embodiments 1-58, wherein the mixture comprising the low solubility molecule, water-miscible solvent, albumin and an agent that prevents self-aggregation of albumin, does not also comprise a solubility-enhancing agent prior to spray-drying.

60. The method according to Embodiment 59, wherein the solubility-enhancing agent is a cyclodextrin, a dispersant, or a surfactant, e.g. polysorbate 80.

61. The method according to any one of Embodiments 1-58, wherein the mixture comprising the low solubility molecule, water-miscible solvent, albumin, an agent that prevents self-aggregation of albumin, also comprises a solubility-enhancing agent prior to spray-drying.

62. The method according to Embodiment 61, wherein the solubility-enhancing agent is a cyclodextrin, a dispersant, or a surfactant, e.g. polysorbate 80.

63. The method according to any one of Embodiments 1-62, wherein the solution or mixture is sterilised prior to spray-drying, optionally wherein one or more of the solutions and/or mixtures is sterilised before or after one or more steps prior to spray-drying, optionally wherein between steps (a) and (b) according to Embodiment 2 the solution of the low solubility molecule and water-miscible solvent is sterilised, and/or between steps (b) and (c) according to Embodiment 2 the mixture is sterilised.

64. The method according to Embodiment 63, wherein the sterilisation of the solution(s) and/or mixture(s) prior to spray-drying is performed by sterile filtration, optionally with a 0.2 μm (micron) sterile filter.

65. The method according to any one of Embodiments 2-64, wherein between steps (b) and (c) given in Embodiment 2, additional water-miscible solvent is added.

66. The method according to Embodiment 65, wherein the additional water-miscible solvent that is added between steps (b) and (c) is ethanol.

67. The method according to any one of Embodiments 1-66, wherein spray-drying the mixture produces a spray-dried composition.

68. A method of preparing a spray-dried composition comprising (i) a Class II or Class IV low solubility molecule, (ii) albumin, and (iii) an agent that prevents self-aggregation of albumin, the method comprising spray-drying a mixture comprising the Class II or Class IV low solubility molecule, a water-miscible solvent, albumin and an agent that prevents self-aggregation of albumin; optionally comprising the steps (a) dissolving the Class II or Class IV low solubility molecule in a water-miscible solvent to form a solution, (b) mixing the solution of the Class II or Class IV low solubility molecule and water-miscible solvent with albumin and an agent that prevents self-aggregation of albumin, and (c) spray-drying the mixture.

69. The method according to Embodiment 67 or 68, wherein the spray-dried composition is suitable for parenteral delivery, subcutaneous delivery, intramuscular delivery, ocular delivery, pulmonary delivery and/or nasal delivery.

70. The method according to any one of Embodiments 67-69, wherein the spray-dried composition does not comprise one or more solubility-enhancing agents.

71. The method according to Embodiment 70, wherein the solubility-enhancing agent is a cyclodextrin.

72. The method according to Embodiment 70 or 71, wherein the solubility-enhancing agent is a surfactant, e.g. polysorbate 80.

73. The method according to any one of Embodiments 67-69, wherein the spray-dried composition comprises one or more solubility-enhancing agents.

74. The method according to Embodiment 73, wherein the solubility-enhancing agent is a cyclodextrin.

75. The method according to Embodiment 73 or 74, wherein the solubility-enhancing agent is a surfactant, e.g. polysorbate 80.

76. The method according to any one of Embodiments 67-75, wherein the method comprises spray-drying the mixture using an outlet temperature of between approximately 40° C. and approximately 120° C., and/or an atomisation pressure of between about 0.5 bar to about 8 bar.

77. The method according to Embodiment 76, wherein the method comprises spray-drying the mixture using an inlet temperature of approximately 100° C., an outlet temperature of approximately 65° C., a liquid feed rate of about 2 ml/min, and an atomisation pressure of about 3 bar.

78. The method according to any one of Embodiments 67-77, wherein the spray-dried composition does not comprise nanoparticles prior to spray-drying.

79. The method according to any one of Embodiments 67-78, wherein the low solubility molecule is as defined in any of Embodiments 3-39.

80. The method according to any one of Embodiments 67-79, wherein the water-miscible solvent is as defined in any of Embodiments 41-43.

81. The method according to any one of Embodiments 67-80, wherein the albumin is as defined in any of Embodiments 44-48, 48-51.

82. The method according to any one of Embodiments 67-81, wherein the agent that prevents self-aggregation of albumin is as defined in any of Embodiments 52-58.

83. The method according to any one of Embodiments 67-81, wherein the ratio of low solubility molecule to albumin is greater than approximately 1:50, or less than 5:1, or between 1:50 and 5:1.

84. The method according to any one of Embodiments 67-83, wherein the solution prior to spray-drying is a single-phase solution of the low solubility molecule, water-miscible solvent, albumin and the agent that prevents self-aggregation of albumin.

85. The method according to any one of Embodiments 67-84, wherein the solution(s) and/or mixture(s) is/are sterilised before or after one or more step(s) prior to spray-drying, as defined in Embodiment 63 or 64.

86. The method according to any one of Embodiments 67-85, wherein between steps (b) and (c) as defined in Embodiment 2 or 68, additional water-miscible solvent is added, as defined in Embodiment 65 or 66.

87. The method according to any one of Embodiments 67-86, wherein substantially all of the albumin within the spray-dried composition is not denatured or crosslinked.

88. The method according to any one of Embodiments 67-87, wherein the method further comprises dissolving the spray-dried composition in aqueous solution.

89. The method according to Embodiment 88, wherein the aqueous solution comprises a surfactant.

90. The method according to Embodiment 89, wherein the surfactant is polysorbate 80.

91. A spray-dried composition comprising (i) a Class II or Class IV low solubility molecule, (ii) albumin, and (iii) an agent that prevents self-aggregation of albumin.

92. Use of albumin in a spray drying method to enhance the solubility and/or the rate of dissolution of a Class II or Class IV low solubility molecule.

93. Use of albumin according to Embodiment 92, wherein the use of albumin is in combination with an agent that prevents self-aggregation of albumin.

94. Use of albumin according to Embodiment 92, or the use of albumin in combination with an agent that prevents self-aggregation of albumin according to Embodiment 93, wherein the use is by carrying out a method according to any one of Embodiments 1-90.

95. A spray-dried composition according to Embodiment 91 for use in medicine.

96. A spray-dried composition according to Embodiment 91 for use in treating or preventing a disease or condition in an individual in need thereof.

1. A method of enhancing the solubility and/or the rate of dissolution of a Class II or Class IV low solubility molecule, the method comprising spray-drying a mixture comprising the Class II or Class IV low solubility molecule, a water-miscible solvent, albumin and an agent that prevents self-aggregation of albumin, optionally wherein the method comprises (a) dissolving the Class II or Class IV low solubility molecule in a water-miscible solvent to form a solution, (b) mixing the solution of the Class II or Class IV low solubility molecule and water-miscible solvent with albumin and an agent that prevents self-aggregation of albumin, and (c) spray-drying the mixture.

2. The method according to claim 1, wherein the low solubility molecule is a peptide, a small molecule, a nucleic acid, a carbohydrate, or a natural product.

3. The method according to claim 1 or 2, wherein the low solubility molecule is selected from the compounds in Table A, optionally wherein the low solubility molecule is a Class II compound selected from the group: aceclofenac, albendazole, atovaquone, bicalutamide, clozapine, danazol, ezetimibe, fenofibrate, glibenclamide, itraconazole, lopinavir, modafinil, nabilone, nimesulide, nimodipine, paliperidone, phenytoin, propofol, prostaglandin E1, rapamycin, repaglinide, risperidone, ritonavir, tacrolimus, teniposide, tretinoin, valsartan, vincristine, voriconazole, zipradisone; or a Class IV compound selected from the group: acyclovir, allopurinol, amoxicillin, amphotericin b, aripiprazole, bifonazole, carfilzomib, cefuroxime axetil, docetaxel, etravirine, linezolid, oxcarbazepine, paclitaxel, rimiducid.

4. The method according to any one of claims 1-3, wherein the water-miscible solvent comprises one or more water-miscible solvents selected from ethanol, acetic acid, acetone, dimethylsulphoxide, formic acid, 1-propanol, 2-propanol, and tetrahydrofuran and N,N-dimethylformamide, preferably wherein the water-miscible solvent(s) comprises ethanol.

5. The method according to any one of claims 1-4, wherein the albumin is a recombinant albumin and/or human albumin.

6. The method according to any one of claims 1-5, wherein the agent that prevents self-aggregation of albumin is a sugar, modified sugar, sugar derivative or polymer.

7. The method according to claim 6, wherein the sugar, modified sugar or sugar derivative is selected from one or more of trehalose, sucrose and dextrose.

8. The method according to claim 7, wherein the sugar, modified sugar or sugar derivative is trehalose.

9. The method according to any one of claims 1-8, wherein spray-drying the mixture produces a spray-dried composition.

10. A method of preparing a spray-dried composition comprising (i) a Class II or Class IV low solubility molecule, (ii) albumin, and (iii) an agent that prevents self-aggregation of albumin, the method comprising spray-drying a mixture comprising the Class II or Class IV low solubility molecule, a water-miscible solvent, albumin and an agent that prevents self-aggregation of albumin; optionally comprising the steps (a) dissolving the Class II or Class IV low solubility molecule in a water-miscible solvent to form a solution, (b) mixing the solution of the Class II or Class IV low solubility molecule and water-miscible solvent with albumin and an agent that prevents self-aggregation of albumin, and (c) spray-drying the mixture.

11. The method according to claim 9 or 10, wherein the spray-dried composition is suitable for parenteral delivery, subcutaneous delivery, intramuscular delivery, ocular delivery, pulmonary delivery and/or nasal delivery.

12. The method according to any one of claims 9-11, wherein the ratio of low solubility molecule to albumin is greater than approximately 1:50, or less than 5:1, or between 1:50 and 5:1.

13. The method according to any one of claims 9-12, wherein the solution prior to spray-drying is a single-phase solution of the low solubility molecule, water-miscible solvent, albumin and the agent that prevents self-aggregation of albumin.

14. A spray-dried composition comprising (i) a Class II or Class IV low solubility molecule, (ii) albumin, and (iii) an agent that prevents self-aggregation of albumin.
